(19) United States
(12) Patent Application Publication
(10) Pub. No.: US 2026/0151538 A1
(43) Pub. Date: Jun. 4, 2026

(54) DIFFERENTIATION AND REPROGRAMMING OF CHONDROCYTE

(71) Applicants: ADA Forsyth Institute, Inc., Cambridge, MA (US); University of Rochester, Rochester, NY (US)

(72) Inventors: Wei Hsu, Cambridge, MA (US); Takamitsu Maruyama, Cambridge, MA (US)

(73) Assignees: ADA Forsyth Institute, Inc., Cambridge, MA (US); University of Rochester, Rochester, NY (US)

(21) Appl. No.: 19/124,176

(22) PCT Filed: Oct. 18, 2023
(86) PCT No.: PCT/US2023/077155

§ 371 Date: Apr. 24, 2025

Related U.S. Application Data
(60) Provisional application No. 63/381,028, filed on Oct. 26, 2022.

(51) Int. Cl. A61L 27/38; C07K 14/47; C12N 5/077; C12N 15/85

(52) U.S. Cl. CPC A61L 27/3817; C07K 14/4702; C12N 5/0655; C12N 15/85; A61L 2430/06; C07K 2319/10; C12N 2501/60; C12N 2506/13; C12N 2510/00

(57) ABSTRACT

This application relates to stem cell biology and regenerative medicine. Disclosed herein are agents and methods for converting non-chondrocyte cells to chondrocytes or chondrogenic ceils, related cells, related compositions, related products, and related uses.

### CROSS-REFERENCE TO RELATED APPLICATION

This application claims priority under 35 U.S.C. § 119(e) to U.S. Provisional Patent Application No. 63/381,028, filed Oct. 26, 2022. The foregoing application is incorporated by reference herein in its entirety.

### GOVERNMENT INTERESTS

This invention was made with government support under DE015654 and DE026936 awarded by the National Institutes of Health. The government has certain rights in the invention.

### REFERENCE TO SEQUENCE LISTING

The present application is being filed along with a Sequence Listing in electronic format. The Sequence Listing is provided as a file titled 346086_00201SeqList.xml, created on Oct. 17, 2023, which is 29,200 bytes in size. The information in the electronic format of the sequence listing is incorporated herein by reference in its entirety.

### FIELD OF THE INVENTION

This invention relates to cell biology and regenerative medicine.

### BACKGROUND

Chondrocytes are cells responsible for cartilage formation. They produce and maintain the cartilaginous matrix, which consists mainly of collagen and proteoglycans, and cover the end of the bone in joints which prevents friction between bones and act as a shock absorber. Loss of the cartilage is responsible for many cases of joint pain and disorders, such as arthritis, temporomandibular joint disorders (TMD), and numerous congenital and cartilage degenerative diseases. For example, osteoarthritis, which is characterized by progressive degradation of articular cartilage leading to loss of joint function, presents the most common musculoskeletal disorder. Approximately 58.5 million Americans (24% of adults) and more than 350 million people worldwide are currently affected while more are expected due to the aging population and an increase in life expectancy. Similarly, 10 million Americans are suffering from TMD. The annual costs for joint-related medical care and lost earnings are $303.5 billion in the US.

Current treatments for cartilage and joint injury as well as related disorders include surgery and other invasive procedures focused on relieving the sings and symptoms but cannot reverse or prevent the progression of the cartilage degeneration. There is a need for novel therapeutic agents and methods for effective cartilage regeneration, treatment of joint damage, and amelioration or prevention of related disorders.

### SUMMARY

This application addresses the need mentioned above in a number of aspects.

In one aspect, the present application provides a method of generating a chondrocyte cell. The method comprises increasing the level of GATA3 in a non-chondrocyte cell. In some embodiments, the increasing step may comprise: (i) contacting the non-chondrocyte cell with a GATA3 activator, (ii) introducing to the non-chondrocyte cell a genetic construct comprising a nucleic acid sequence encoding GATA3, or (iii) introducing to the non-chondrocyte cell a GATA3 polypeptide. The non-chondrocyte cell can be any suitable cell.

In some embodiments, the non-chondrocyte cell is a mesenchymal stem cell (MSC), a suture stem cell (SuSC), a skeletal stem cell (SSC), or a bone cartilage and stomal progenitor (BCSP). In other embodiments, the non-chondrocyte cell is a somatic cell, a fibroblast, a skin cell, a muscle cell, an epithelium cell, a blood cell, a neuron, an embryonic cell, or a pluripotent stem cell (iPSC). The method can further comprise culturing the non-chondrocyte cell or its progeny under conditions permitting differentiation of chondrocyte to obtain one or more progeny cells thereof, thereby generating chondrocyte cells.

In another aspect, the present application provides a cultured recombinant cell comprising (i) a heterologous GATA3 polypeptide or (ii) a heterologous nucleic acid encoding a GATA3 polypeptide, or a progeny cell thereof. Also provided is a cell obtained according to the method described above or a progeny cell thereof. In a further aspect, the present application provides a composition comprising (i) the cell described above or a cell derived therefrom, and (ii) a carrier. The composition can be a pharmaceutical composition and in that case, the carrier can be pharmaceutically acceptable.

In another aspect, the present application provides a cartilage regeneration product or artificial cartilage. The cartilage regeneration product or artificial cartilage comprises (i) the cell described above or progeny cell thereof, or the composition described above, and (ii) a scaffold.

In another aspect, the present application provides a method of generating or regenerating a cartilaginous tissue at a site in a subject in need thereof. The method comprises: (i) increasing a level of GATA3 in one or more cells at or around the site; or (ii) administering to the site: the above-described cell or progeny thereof, or the above-described composition, or the above-described cartilage regeneration product or artificial cartilage.

In a further aspect, the present application provides a method of treating cartilage damage or a method of regenerating cartilage at a site in a subject in need thereof. The method comprises: (i) increasing a level of GATA3 in one or more cells at or around the site; or (ii) administering to the site, the above-described cell or progeny thereof, or the above-described composition, or above-described cartilage regeneration product or artificial cartilage. In one example, the subject has arthritis or an injury at the site. Arthritis can be osteoarthritis, rheumatoid arthritis, childhood arthritis, fibromyalgia, gout, and lupus. Preferably, the method enhances healing of the cartilage damage or/and prohibits the development of fibrous connective tissue or fibrosis.

In each of the methods described above, the increasing step may comprise administering to the site a GATA3 activator, a genetic construct comprising a nucleic acid sequence encoding GATA3 or a GATA3 polypeptide Examples of the GATA3 activator include Gemfibrozil. SCH-58261, rosiglitazone, WY-14643, SB202190, U0126, docosahexaenoic acid (DHA), and Jagged1. In some embodiments, the GATA3 polypeptide may be linked to a cell-penetrating moiety. The method may further comprise administering to the subject a chondrogenic factor, such as vitamin D3, collagen hydrolyzate, FGFs, BMPs, a steroid, or a non-steroidal anti-inflammatory agent (NSAID). The subject can be a mammal, such as a human. The cell can be heterologous, xenogenic, allogeneic, isogenic, or autologous to the subject.

In another aspect, the present application provides a kit for generating/regenerating cartilaginous tissue, or for treating cartilage damage. The kit comprises one, two, or more selected from a group consisting of a GATA3 activator, a genetic construct comprising a nucleic acid sequence encoding GATA3, a GATA3 polypeptide, the above-described cell or progeny thereof, the above-described composition, and the above-described cartilage regeneration product or artificial cartilage. In some examples, the kit may further comprise a scaffold or a chondrogenic factor.

As the cartilage in endochondral ossification and fracture healing is necessary for bone formation, the above-described methods, cells, compositions, activators, products, and kits may also be useful for related bone formation. Accordingly, within the scope of this disclosure is a method for bone formation at a site in a subject in need thereof. The method comprises: (i) increasing a level of GATA3 in one or more cells at or around the site; or (ii) administering to the site: the above-described cell or progeny thereof, or the above-described composition, or above-described cartilage regeneration product or artificial cartilage.

The details of one or more embodiments of the present application are outlined in the description below. Other features, objectives, and advantages of the present application will be apparent from the description and the claims.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIGS. 1A, 1B, 1C, 1D, 1E, 1F, 1G, 1H, 1I, 1J, 1K, 1L, 1M, 1N, and 1O show examination of Wnt signaling and β-catenin-dependent transcription in skeletal cell fate determination. Coronal sections of control (A, I; genotype: β-caleninFz/Fx and Dermol-Cre+; β-cateninFx/+, D; genotype: Gpr177Fx/Fx, G; genotype: R26StopWnt5aFx/+), β-cat^{Dermol} (B, J; genotype: β-cateninFx/Fx; Dermol-Cre+/−). Gpr177^{Dermol} (C; genotype: Gpr177Fx/Fx; Dermol-Cre+/−), Gpr177^{K14} (E; genotype: Gpr177Fx/Fx; K14-Cre+), Gpr177^{Dermol/K14} (F; genotype: Gpr177Fx/Fx; Dermol-Cre+/−; K14-Cre+), Wnt5a^{Dermol-OE} (H; genotype: R26StopWnt5aFx/+; Dermol-Cre+/−), and β-cat^{Dermol}ΔTF (K; genotype: β-catenin^{dm}/Fx, Dermol-Cre+/−) skulls are examined by double labeling of von Kossa (vK) and alcian blue (AB) at E15.5 (A-K). All sections are counterstained by nuclear fast red. Note bone ossification is stained by vK and ectopic chondrogenesis is only detected in the 0-cat^{Dermol} mutants (B, J). Asterisks indicate the site of skeletal stem cells switching from osteoblast to chondrocyte fate. Broken lines define the skeletogenic mesenchyme. Images are representatives of at least three independent experiments. (L-M, O) Graphs show quantitation of the average percentage of the positively stained area (mm²) over the calvarial mesenchymal area (mm²) from three mice per group and are presented as p-value<0.001, means±SD, two-sided student t-test. (N) No significant difference between the vK+ area in control and Wnt5a^{Dermol-OE}. Scale bars, 200 μm (A-K).

FIGS. 2A, 2B, 2C, and 2D show that β-catenin-dependent transcription is essential for osteoblastogenesis but not skeletal lineage specification. Coronal sections of E15.5 control (genotype: β-cateninFx/Fx), β-cat^{Dermol}, and β-cat^{Dermol} ΔTF are analyzed by in situ hybridization of Col2 (A), Col1 (C), and osteocalcin (OC; D) and counterstained by nuclear fast red, and immunostaining of Osterix (Osx) and counterstained by hematoxylin (B). Graphs show quantitation of the average percentage of the positively stained area (mm²) over the calvarial mesenchymal area (mm²) from three mice per group (*, p-value<0.001; **, p-value<0.02 means±SD, two-sided student t-test). Images are representatives of three independent experiments. Scale bar, 200 μm (A-D). FIGS. 3A, 3B, 3C, 3D, 3E, and 3F show disruption of transcription but not cell adhesion of β-catenin in the skeletogenic mesenchyme of β-cat^{Dermol}ΔTF. Coronal sections of the control (genotype: β-cateninFx/Fx), β-cat^{Dermol}, and β-cat^{Dermol}ΔTF calvaria are examined by immunostaining using antibodies recognizing the N-terminal (α-βcat N; A) or C-terminal (α-βcat C; B) domain of β-catenin. OB-cadherin (OB-Cad; C). LEF1 (D). TCF7 (E), or DKK1 (F) at E15.5. Broken lines define the skeletogenic mesenchyme and Br indicates the brain. Images are representatives of three independent experiments. Scale bars, 100 μm (A-E); 200 μm (F).

FIGS. 4A, 4B, 4C, 4D, and 4E show an assessment of skeletal lineage commitment by stem cell transplantation. Kidney capsule transplantation of approximately 5×10⁴ control (β-cateninFx/Fx), β-cat-null (β-cateninFx/Fx plus lentivirus-Cre), and β-catΔTF (β-catenin^{dm}/Fx plus lentivirus-Cre) cells, isolated from the P5 calvarial suture mesenchyme, examine the stem cell property. The transplants are examined two weeks after transplantation. Ectopic bone generated by the transplanted stem cells is assessed by hematoxylin and eosin (H&E) staining (A) and immunostaining of Osx (B). The presence of chondrocytes is determined by alcian blue (AB) staining (C), and immunostaining of Acan (D) and Col2 (E). Note that β-cat-null stem cells alter their fate and develop into chondrocytes instead of osteoblasts. No detection of osteoblasts or chondrocytes in the β-catΔTF transplant. Images are representatives of three independent experiments (100% transplantation success rate). Scale bars, 100 mm (A-E).

FIGS. 5A, 5B, and 5C show gene expression profiling of classical and nonclassical β-catenin signaling. (A) Diagrams illustrate a strategy to identify differentially expressed genes (DEGs) for skeletal fate determination and OB differentiation linked to nonclassical signaling effects of β-catenin (unique DEGs) and canonical Wnt/β-catenin signaling (common DEGs), respectively. SC, skeletal cell; OB, osteoblast. Heatmap showing the expression of osteoblast (B) and chondrocyte (C) genes associated with the β-catenin mutations identified by gene expression profiling of the calvarial mesenchyme using RNA-seq analysis. Acan, aggrecan; Col1a1, Collagen type I α1; Col2a1, Collagen type II α1; (Col10a1, Collagen type X α1; Ibsp. Bone Sialoprotein 2; OPN, Osteopontin; OCN, Osteocalcin; Osx, Osterix; Runx2, Runt-related transcription factor 2.

FIGS. 6A, 6B, 6C, and 6D show the identification of GATA3 mediating the effect of nonclassical β-catenin signaling. (A) Diagrams illustrate a strategy to identify upstream regulators in the alternative pathway mediating the primary effects based on the statistical over-connection in the DEGs. (B) GATA3 is the top candidate identified by an Interactome/Upstream Regulator analysis to mediate the effect of β-catenin but independent of canonical Wnt signaling using MetaCore. (C) Immunostaining of the E15.5 control (genotype: β-cateninFx/Fx), β-cat^{Dermol}, and β-cat^{Dermol}ΔTF calvarial sections using anti-Gata3 antibodies. Broken lines define the skeletogenic mesenchymal region. (D) Double-labeling indicates GATA3 expression in AB-positive chondrocytes. Arrows and insets show ectopic cartilages. Br, brain; Ep, epidermis; SM, skeletogenic mesenchyme. Scale bars, 200 μm (C); 100 μm (D).

FIGS. 7A, 7B, and 7C show chondrogenic lineage specification and chondrogenesis mediated by GATA3. (A) In vitro culture of C3H10T1/2 cells is infected by lentiviruses expressing GFP (control) or mouse Gata3 (Gata3^{OE}) in the chondrogenic condition, followed by AB staining and quantification of the AB-positive area (Mean±SD, n=3, p-value<0.05). (B, C) Whole-mount AB staining of E18.5 control (B; genotype: R26StopGATA3+/−) and GATA3^{Dermol-OE} (C; genotype: R26StopGATA3+/−; Dermol-Cre+) skulls reveals aberrant chondrogenesis caused by overexpression of GATA3 in skeletogenic mesenchyme. Asterisks indicate sites of aberrant chondrogenesis. Images are the representatives of at least three independent experiments. Scale bars, 200 μm (A); 2 mm (B, C); 500 μm (B1-B3 and C1-C3).

FIGS. 8A, 8B, and 8C show that GATA3 alters the commitment of skeletogenic mesenchyme in calvarial morphogenesis. (A-C) Coronal sections of E18.5 control (genotype: R26StopGATA3+/−) and GATA3^{Dermol-OE} (genotype: R26StopGATA3+/−; Dermol-Cre+) skulls are analyzed by AB staining, immunostaining of Acan and Osx, and double labeling of AB and GATA3. Arrows and arrowheads indicate ectopic chondrogenesis in the calvarial sutures and drastic expansion of nasal cartilage, respectively. Images are the representatives of three independent experiments.

FIGS. 9A, 9B, and 9C show GATA3 in mediating nonclassical β-catenin signaling for skeletal cell fate determination. (A) Micromass culture of primary cells isolated from control (genotype: β-cateninFx/Fx) and (β-cat^{Dermol} infected by the lentivirus scramble or shGata3 (shRNA-mediated knockdown of Gata3) in the chondrogenic condition, followed by AB staining 7 days after culture (B) Graphs show quantification of the AB-positive area (Mean±SD, n=3 animals, p-value<0.01, two-sided student t-test). (C) Quantitative RT-PCR examines Sox9, Acan, and Col2a1 expression modulated by β-catenin and Gata3 (Mean t SD, n=3 animals, p-value<0.01, two-sided student 1-test). Scale bars, 2 mm (A).

FIGS. 10A and 10B show the effects of the loss of Gpr177/mouse Wntless and β-catenin on mandibular development. Double labeling of von Kossa (vK) and alcian blue (AB) examines coronal sections of control (genotype: Gpr177Fx/Fx). Gpr177^{Dermol} (genotype: Gpr177Fx/Fx; Dermol-Cre+/−). Gpr177^{K14} (genotype: Gpr177Fx/Fx; K14-Cre+), Gpr177^{Dermol/K14} (genotype: Gpr177Fx/Fx; Dermol-Cre+/−; K14-Cre+) skulls (A) and control (genotype: Gpr177Fx/Fx), β-cat^{Dermol} (β-cateninFx/Fx; Dermol-Cre+/−), and β-cat^{Dermol}ΔTF (Δ-catenin^{dm}/Fx; Dermol-Cre+/−) skulls at E15.5 (B). Sections are counterstained by nuclear fast red. Images are the representatives of three independent experiments. MC, Meckel's Cartilage. Scale bars, 200 μm (A. B).

FIGS. 11A and 11B show the examination of gene deletion and expression efficiency in mutant mouse models of Gpr177 and Wnt5a. (A) The efficiency of Gpr177 disruption is determined by immunostaining analysis of the control (genotype: Gpr177Fx/Fx), Gpr177^{Dermol} (genotype: Gpr177Fx/Fx; Dermol-Cre+/−), Gpr177^{K14} (genotype: Gpr177Fx/Fx; K14-Cre+), Gpr177^{Dermol/K14} (genotype: Gpr177Fx/Fx; Dermol-Cre+/−; K14-Cre+). (B) The transgenic expression of Wnt5a is examined by immunostaining analysis of the control (genotype: R26StopWnt5aFx/+) and Wnt5a^{Dermol-OE} (genotype: R26StopWnt5aFx/+; Dermol-Cre+/−). Sections are counterstained by DAPI. Images are representatives of three independent experiments. Scale bars, 200 μm (A, B).

FIGS. 12A, 12B, and 12C show the effects of Gpr177 deficiency and Wnt5a overexpression on the development of the limb. (A, B) Double labeling of vK and AB examines sections of the control (genotype: Gpr177Fx/Fx), Gpr177^{Dermol} (genotype: Gpr177Fx/Fx; Dermol-Cre+/−). Gpr177^{K14} (genotype: Gpr177Fx/Fx; K14-Cre+), Gpr177^{Dermol/K14} (genotype: Gpr177Fx/Fx; Dermol-Cre+/−; K14-Cre−) femurs at E15.5. Sections are counterstained by nuclear fast red. Note mesenchymal but not epithelial deletion of Gpr177 affects endochondral ossification with delayed chondrogenesis and formation of primary ossification center. No ectopic chondrogenesis is detected in all mutants. (C) Double labeling of vK and AB examining sections of the control (genotype: R26StopWnt5aFx/+) and Wnt5a^{Dermol-OE} (H; genotype: R26StopWnt5aFx/+; Dermol-Cre+/−) femurs reveals no abnormality in ossification and chondrogenesis associated with overexpression of Wnt5a in developing limbs Scale bars, 500 μm (A. C); 200 μm (B).

FIGS. 13A, 13B, 13C, and 13D show the efficiency of lentiviral infection and gene deletion in kidney capsule transplantation. (A) Whole-mount analysis of the kidney without (No cells) or with transplantation of suture cells (Cells), including the infection of lentivirus-Cre-RFP (Cell+Cre-RFP). The presence of virus-infected cells expression Cre and RFP is visualized by superimposed fluorescent and bright-field images. (B-D) Sections of the kidney without (No cells) or with transplantation of 5×10⁴ cells isolated from wild-type suture mesenchyme (Control). The transplantation of β-cateninFx-Fx and β-catenin^{dm}/Fx suture cells infected by lentivirus-Cre-RFP is indicated as β-cat-null and β-catΔTF, respectively. (B) Bright-field images show the kidney capsules without (No cells) or with the presence of transplanted cells that show enlarged capsule regions containing bone (Control), cartilage (β-cat-null), and non-bone/cartilage (β-catΔTF). (C) RFP and (D) β-catenin staining analyses of the control. β-cat-null and (β-catΔTF sections, counterstained by DAPI. The RFP reporter represents cells infected by lentivirus. The β-catenin+ cells are present in the bone generated by control cells. Arrows indicate the cartilage generated by β-cat-null cells. K, kidney; KC, kidney capsule. Scale bar, 500 μm (A); 200 μm (B, C); 100 μm (D).

FIGS. 14A, 14B, 14C, 14D, and 14E show that β-catenin-mediated transcription is essential for endochondral ossification during limb development. Sections of the E15.5 control (genotype: β-cateninFx/Fx), β-cat^{Dermol}, and β-cat^{Dermol}ΔTF humeri are analyzed by double labeling of AB and vK (A), immunostaining of Col2 (B) and Osx (D), and in situ hybridization of Col10 (C) and Col1 (E) Images are representatives of three independent experiments. Scale bars, 200 μm (A-E).

FIG. 15 shows the requirement of β-catenin-dependent transcription for palatogenesis. Coronal sections of E18.5 control (genotype: β-cateninFx/Fx), β-cat^{Dermol}, and β-cat^{Dermol}ΔTF skulls are examined by hematoxylin and eosin staining. Enlargements of the insets are shown in the bottom panels. Note both β-cat^{Dermol}, and β-catenin^{Dermol}ΔTF mutants exhibit cleft palate. Images are the representatives of three independent experiments. Scale bars, 200 μm (top panels), 100 μm (bottom panels).

FIGS. 16A and 16B show effects of the β-catenin mutations on canonical Wnt signaling. (A) A table showing the top 50 pathways that are affected by both mutations in the calvarial mesenchyme based on Pathway Map analysis of β-cat^{Dermol} and β-cat^{Dermol}ΔTF common DEGs using MetaCore. (B) Heatmap showing a list of known canonical Wnt downstream genes that are direct transcriptional targets of β-catenin-LEF/TCF downregulated in the β-cat^{Dermol} and β-cat^{Dermol}ΔTF calvarial mesenchyme.

FIG. 17 shows the top candidates of the upstream regulators in nonclassical β-catenin signaling. Actual represents the number of network objects in the activated dataset(s) which interact with the chosen object listed in the gene name column. n; the number of network objects in the dataset; R, the number of network objects in the complete database or background list that interact with the chosen object; N, the total number of gene-based objects in the complete database or background list; Expected, mean of the hypergeometric distribution (n·R/N); Ratio, the connectivity ratio (Actual/Expected), p-value=probability to have the given or higher (lower for negative z-score) value of Actual by chance under the null hypothesis of no over or under-connectivity.

FIGS. 18A and 18B show the mouse genetic model for Dermol-Cre-mediated expression of GATA3 in the skeletogenic mesenchyme. (A) Schematic of the strategy for transgenic expression of GATA3 in the developing skeletogenic mesenchyme using Dermol-Cre and R26StopGATA3 alleles. (B) Immunostaining of E18.5 control (genotype: R26StopGATA3Fx/+) and GATA3^{Dermol-OE} (genotype: R26StopGATA3Fx/+; Dermol-Cre+/−) skull coronal sections showed elevated levels of human GATA3 expression in the calvarial suture mesenchyme. Enlargements of the insets are exhibited on the right panels. Note the ectopic cartilage shown in the GATA3^{Dermol-OE} mutant expressing GATA3 (middle row). Images are the representatives of three independent experiments. Scale bars, 500 μm (A, B).

FIGS. 19A, 19B, 19C, and 19D show the switching of skeletal precursor cells to a chondrogenic fate and initiation of ectopic chondrogenesis b. GATA3. (A) Coronal sections of E15.5 (A. C) and E16.5 (B. D) control (genotype. R26StopGATA3Fx/+) and GATA3^{Dermol-OE} (genotype R26StopGATA3Fx/+; Dermol-Cre+/−) skulls are examined by AB staining (A, B), and immunostaining of Acan (C. D). Arrowheads and arrows indicate drastic expansion of nasal cartilage and ectopic chondrogenesis in the calvarial sutures, respectively. Images are the representatives of three independent experiments. Scale bars, 200 μm (A, B, C); 50 μm (D).

FIGS. 20A and 20B show no effects of chondrogenesis by GATA3 overexpression in the developing limb. Double labeling of vK and AB examines sections of the control (A; genotype: R26StopGATA3Fx/+), and GATA3^{Dermol-OE} (genotype: R26StopGATA3Fx/+; Dermol-Cre+/−) femurs and tibias at E15.5. Sections are counterstained by nuclear fast red. No ectopic chondrogenesis is detected in all mutants. Scale bars, 500 μm (A, B).

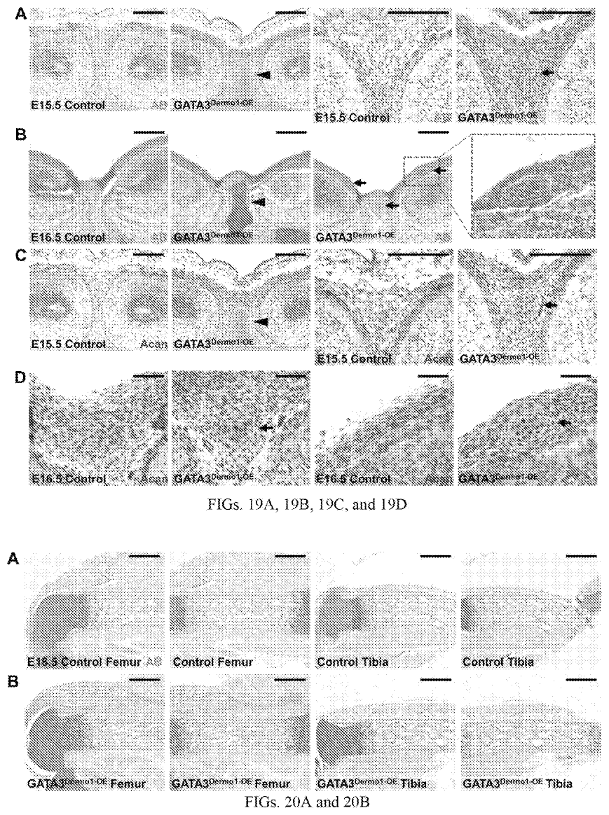

FIG. 21 shows docosahexaenoic acid promotes chondrocyte differentiation. Images show alcian blue (AB) staining of ATDC5 cells cultured in micro-mass with the presence of 0, 1, 5, 25, and 125 μM DHA. The graph showing the quantitation analysis of chondrogenesis examines the percentage of AB positively stained area increased by DHA in a dosage-dependent effect.

FIG. 22 shows the effect of GATA activators on chondrogenesis. Graphs showing the quantitation analysis of chondrocyte differentiation of ATDC5 cells examine the percentage of AB positively stained area affected by Pirinixic acid, Gemfibrozil, SB 202190, SCH 58261, and U0126.

### DETAILED DESCRIPTION OF THE INVENTION

This disclosure relates to stem cell biology and regenerative medicine. Some aspects of this disclosure are based, at least in part, on unexpected discoveries of the ability of GATA3 to convert non-chondrocyte cells to chondrocytes or chondrogenic cells. Certain aspects of this disclosure are also based on discoveries that skeletal cell fate is determined by β-catenin but independent of LEF/TCF transcription, that GATA3 is a mediator for the alternative signaling effects, and more significantly that GATA3 alone is sufficient to promote ectopic cartilage formation. The abilities of GATA3 to promote the chondrocyte lineage fate commitment and to transdifferentiate a non-chondrocyte cell into a chondrocyte can be used for cell-based therapy.

### Signaling Pathways and Cell Fate/Lineage Commitment

Some aspects of this disclosure related to signaling pathways, such as that of Wnt signaling, in cell fate or lineage commitment. For example, skeletal precursors, which are mesenchymal in origin, can give rise to distinct sublineages. Their lineage commitment is modulated by various signaling pathways. The importance of Wnt signaling in skeletal lineage commitment has been implicated by the study of β-catenin deficient mouse models. Ectopic chondrogenesis caused by the loss of β-catenin leads to a long-standing belief in canonical Wnt signaling determines skeletal cell fate. As β-catenin possesses other functions, it remains unclear if skeletogenic lineage commitment is solely orchestrated by canonical Wnt signaling. The study of the Wnt secretion regulator—Gpr177/Wntless also raises concerns about current knowledge.

As disclosed herein, skeletal cell fate is determined by β-catenin but independent of LEF/TCF transcription. Further, genomic and bioinformatic analyses identify GATA3 as a mediator for the alternative signaling effects. Moreover. GATA3 alone is found to be sufficient to promote ectopic cartilage formation, demonstrating its essential role in mediating nonclassical β-catenin signaling in skeletogenic lineage specification.

Lineage specification is pertinent to the creation of an organism. In mammalian embryos, the first two distinct lineages to form are the outer trophectoderm and the inner cell mass of the blastocyst (1). Subsequently, three germ layers are formed followed by the development of the fourth germ layer—the neural crest (2, 3). Their dynamic interactions via molecular signals in the form of proteins, RNAs, surface contacts, and mechanics modulate the commitment of each cell and its neighboring cells to form diverse lineages and specified cell types during organogenesis (4). Three distinct lineages, somites, lateral plate mesoderm, and cranial neural crest, give rise to the axial skeleton, limb skeleton, and craniofacial bone and cartilage, respectively (5). Studies of the origin of cells that generate these tissues have led to the isolation and characterization of skeletogenic/skeletal stem cells (6-14). Recent advancements in stem cell research further offer next-generation therapeutics for large craniofacial defects caused by various conditions, including trauma, infection, tumors, congenital disorders, and progressive deforming diseases (12, 15). Proper cell fate determination can further facilitate the efficacy of stem cell-based therapy.

Cell fate switching has been linked to the pathogenesis of human diseases. Activation of canonical Wnt signaling plays a crucial role in muscle stem cell conversion from a myogenic to a fibrogenic lineage in aging mice (16). Later evidence suggests the Wnt/TGFβ-mediated lineage conversion promotes muscle stem cells to acquire fibroblast phenotypes, leading to muscular dystrophy (17). Heterotopic ossification is another example of cell fate switching as a pathogenic cause (18). The transformation of primitive cells in mesenchymal origin into osteogenic cells results in bone formation within the soft connective tissue. Cell fate switching is the most commonly triggered by traumatic injury—the acquired form (19). However, there is also a rare congenital form—Fibrodysplasia Ossificans Progressiva (FOP) linked to the autosomal dominant mutation in BMP type I receptor ACVR1 (20). Furthermore, the interplay of BMP and FGF signaling is modulated by Wnt in stem cell-mediated intramembranous ossification during calvarial morphogenesis (21). Disrupting the balance of this signaling crosstalk can alter the stem cell from osteogenic to chondrogenic fate, leading to aberrant endochondral ossification and craniosynostosis (21).

The requirement of canonical Wnt signaling in skeletal lineage commitment is based on the disruption of β-catenin causing ectopic chondrogenesis in mice (22, 23). The mouse genetic study of Lrp5 and Lrp6 further supports the role of canonical Wnt signaling in the promotion of osteoblast fate (24). However, multiple functions of β-catenin and no cell fate alteration, detected by the loss of Gpr177/mouse Wntless, raise concerns about this theory (25). Therefore, the inventors' group has created several mouse models to examine details of the skeletal cell fate decision mediated by β-catenin. Related findings provide evidence supporting an alternative mechanism mediated by β-catenin independent of the transcriptional output of canonical Wnt signaling is necessary for inhibiting ectopic chondrogenesis.

As disclosed herein, the whole genomics study examines downstream effectors, leading to the identification of GATA3 as the key modulator associated with these alternative signaling effects of β-catenin. Functional analyses further demonstrate that the GATA3 transcription factor is sufficient to promote the commitment of skeletogenic mesenchyme to chondrocyte lineage. GATA3 mediates the downstream effects of β-catenin on switching the fate of skeletal precursor cells.

### GATA3

GATA3 is a transcription factor, which in humans is encoded by the GATA3 gene. It belongs to the GATA family of transcription factors containing the zinc figure motif that recognizes G-A-T-A nucleotide sequences to activate or repress target genes (M. Trembla et al., Development (Cambridge, England) 145, (2018)). GATA3 regulates or controls the expression of a wide range of biologically and clinically important genes. It is critical for the embryonic development of various tissues as well as for inflammatory and humoral immune responses and the proper functioning of the endothelium of blood vessels. GATA3 plays a central role in allergy and immunity against worm infections.

Germline deletion of Gata3 in mice causes embryonic lethality and exhibits a variety of phenotypes in several tissues including craniofacial bone and cartilage (P. P. Pandolfi et al., Nature genetics 11, 40-44 (1995) and K. C. Lim et al., Nature genetics 25, 209-212 (2000). In humans, the haploinsufficiency of GATA3 has been linked to HDR syndrome and craniofacial defects (H. Van Esch et al., Nature 406, 419-422 (2000)). Genomewide association study further has resealed susceptibility loci for craniofacial microsomia, leading to the identification of mutations in GATA3 (Y. B. Zhang et al. Nature communications 7, 10605 (2016)). GATA3 is also known to regulate various stages of hematopoiesis both in the adult and during development (N. Zaidan, et al., Open Biol 8. (2018)). It has multiple functions with a role in developing the first hematopoietic stem cells in the embryo. GATA3 is also proposed to be a clinically important marker for various types of cancer, particularly those of the breast. However, the role, if any, of GATA3 in the development of these cancers is under study and remains unclear.

The findings disclosed herein support GATA3 in the control of key lineage-specific factors to drive the cell fate decision and tissue morphogenesis. This is attributed to GATA proteins belonging to a subclass of pioneer transcription factors capable of promoting chromatin opening and recruitment of additional transcriptional regulators (K. S. Zaret et al, Genes & development 25, 2227-2241 (2011)). In addition to their pioneer activity. GATA factors also possess three-dimensional chromatin reorganization ability (M. Trembla et al. Development (Cambridge, England) 145, (2018)). Several GATA members act as primary regulators of various lineage decisions and cell fate determinations (M. Trembla et al., Development (Cambridge, England) 145, (2018)). Further deciphering its orchestral influence at the chromatin level promises important insights into the action of GATA3 as a master chondrogenic regulator.

Various GATA3 protein and nucleic acid sequences are known. Exemplary GATA3 genomic sequence includes human GATA3 genomic sequence as annotated under GenBank accession number NG 015859.1. Exemplary GATA3 mRNA includes human GATA3 mRNA having the nucleic acid sequence as annotated under GenBank accession numbers NM 001002295.1 (isoform 1), or NM 002051.2 (isoform 2). Exemplary GATA3 protein includes human GATA3 protein having the amino acid sequence as annotated under GenBank accession number NP 001002295.1 (isoform 1) or NP 002042.1 (isoform 2). Shown below are some exemplary amino acid sequences and nucleic acid sequences.

GATA3 BC003070
(SEQ ID NO: 2)
GCGAGACAGAGCGAGCAACGCAATCTGACCGAGCAGGTCGTACGCCGCCGCCTCCTCCTCCTCTCTGCTCT
TCGCTACCCAGGTGACCCGAGGAGGGACTCCGCCTCCGAGCGGCTGAGGACCCCGGTGCAGAGGAGCCTGG
CTCGCAGAATTGCAGAGTCGTCGCCCCTTTTTACAACCTGGTCCCGTTTTATTCTGCCATACCCAGTTTTT
GGATTTTTGTCTTCCCCTTCTTCTCTTTGCTAAACGACCCCTCCAAGATAATTTTTAAAAAACCTTCTCCT
TTGCTCACCTTTGCTTCCCAGCCTTCCCATCCCCCCACCGAAAGCAAATCATTCAACGACCCCCGACCCTC
CGACGGCAGGAGCCCCCCGACCTCCCAGGCGGACCGCCCTCCCTCCCCGCGCGCGGGTTCCGGGCCCGGCG
AGAGGGCGCGAGCACAGCCGAGGCCATGGAGGTGACGGCGGACCAGCCGCGCTGGGTGAGCCACCACCACC
CCGCCGTGCTCAACGGGCAGCACCCGGACACGCACCACCCGGGCCTCAGCCACTCCTACATGGACGCGGCG
CAGTACCCGCTGCCGGAGGAGGTGGATGTGCTTTTTAACATCGACGGTCAAGGCAACCACGTCCCGCCCTA
CTACGGAAACTCGGTCAGGGCCACGGTGCAGAGGTACCCTCCGACCCACCACGGGAGCCAGGTGTGCCGCC
CGCCTCTGCTTCATGGATCCCTACCCTGGCTGGACGGCGGCAAAGCCCTGGGCAGCCACCACACCGCCTCC
CCCTGGAATCTCAGCCCCTTCTCCAAGACGTCCATCCACCACGGCTCCCCGGGGCCCCTCTCCGTCTACCC
CCCGGCCTCGTCCTCCTCCTTGTCGGGGGGCCACGCCAGCCCGCACCTCTTCACCTTCCCGCCCACCCCGC
CGAAGGACGTCTCCCCGGACCCATCGCTGTCCACCCCAGGCTCGGCCGGCTCGGCCCGGCAGGACGAGAAA
GAGTGCCTCAAGTACCAGGTGCCCCTGCCCGACAGCATGAAGCTGGAGTCGTCCCACTCCCGTGGCAGCAT
GACCGCCCTGGGTGGAGCCTCCTCGTCGACCCACCACCCCATCACCACCTACCCGCCCTACGTGCCCGAGT
ACAGCTCCGGACTCITCCCCCCCAGCAGCCTGCTGGGCGGCTCCCCCACCGGCTTCGGATGCAAGTCCAGG
CCCAAGGCCCGGTCCAGCACAGGCAGGGAGTGTGTGAACTGTGGGGCAACCTCGACCCCACTGTGGCGGCG
AGATGGCACGGGACACTACCTGTGCAACGCCTGCGGGCTCTATCACAAAATGAACGGACAGAACCGGCCCC
TCATTAAGCCCAAGCGAAGGCTGTCTGCAGCCAGGAGAGCAGGGACGTCCTGTGCGAACTGTCAGACCACC
ACAACCACACTCTGGAGGAGGAATGCCAATGGGGACCCTGTCTGCAATGCCTGTGGGCTCTACTACAAGCT
TCACAATATTAACAGACCCCTGACTATGAAGAAGGAAGGCATCCAGACCAGAAACCGAAAAATGTCTAGCA
AATCCAAAAAGTGCAAAAAAGTGCATGACTCACTGGAGGACTTCCCCAAGAACAGCTCGTTTAACCCGGCC
GCCCTCTCCAGACACATGTCCTCCCTGAGCCACATCTCGCCCTTCAGCCACTCCAGCCACATGCTGACCAC
GCCCACGCCGATGCACCCGCCATCCAGCCTGTCCTTTGGACCACACCACCCCTCCAGCATGGTCACCGCCA
TGGGTTAGAGCCCTGCTCGATGCTCACAGGGCCCCCAGCGAGAGTCCCTGCAGTCCCTTTCGACTTGCATT
TTTGCAGGAGCAGTATCATGAAGCCTAAACGCGATGGATATATGTTTTTGAAGGCAGAAAGCAAAATTATG
TTTGCCACTTTGCAAAGGAGCTCACTGTGGTGTCTGTGTTCCAACCACTGAATCTGGACCCCATCTGTGAA
TAAGCCATTCTGACTCATATCCCCTATTTAACAGGGTCTCTAGTGCTGTGAAAAAAAAAAAATGCTGAACA
TTGCATATAACTTATATTGTAAGAAATACTGTACAATGACTTTATTGCATCTGGGTAGCTGTAAGGCATGA
AGGATGCCAAGAAGTTTAAGGAATATGGGAGAAATAGTGTGGAAATTAAGAAGAAACTAGGTCTGATATTC
AAATGGACAAACTGCCAGTTTTGTTTCCTTTCACTGGCCACAGTTGTTTGATGCATTAAAAGAAAATAAAA
AAAAGAAAAAAGAGAAAAAAAAAAAAAAAAAAAA
GATA3 Peptide
(SEQ ID NO: 3)
MEVTADQPRWVSHHHPAVLNGQHPDTHHPGLSHSYMDAAQYPLPEEVDVLFNIDGQGNHVPPYYGNSVRAT
VQRYPPTHHGSQVCRPPLLHGSLPWLDGGKALGSHHTASPWNLSPFSKTSIHHGSPGPLSVYPPASSSSLS
GGHASPHLFTFPPTPPKDVSPDPSLSTPGSAGSARQDEKECLKYQVPLPDSMKLESSHSRGSMTALGGASS
STHHPITTYPPYVPEYSSGLFPPSSLLGGSPTGFGCKSRPKARSSTGRECVNCGATSTPLWRRDGTGHYLC
NACGLYHKMNGQNRPLIKPKRRLSAARRAGTSCANCQTTTTTLWRRNANGDPVCNACGLYYKLHNINRPLT
MKKEGIQTRNRKMSSKSKKCKKVHDSLEDFPKNSSFNPAALSRHMSSLSHISPFSHSSHMLTTPTPMHPPS
SLSFGPHHPSSMVTAMG

The above human sequences were used in the assays described in the examples below. The segment that is underlined and in bold (SEQ ID NO: 1), is expected to have the same activity as that of the full length. In addition to these sequences, many other human or mouse GATA3 protein variants are known and can be used in the same or similar manner disclosed herein. Some examples are shown below.

TABLE 1
Human GATA3 variants

| | | | | | SEQ ID | SEQ ID |
|---|---|---|---|---|---|---|
| | | | | | NO. | NO. |
| | | | | | (nucleic | (amino |
| Transcript ID | Name | bp | Protein | Translation ID | acid) | acid) |
| ENST00000379328.9 | GATA3-202 | 3083 | 444aa | ENSP00000368632.3 | 4 | 5 |
| ENST00000346208.4 | GATA3-201 | 2650 | 443aa | ENSP00000341619.3 | 6 | 7 |
| ENST00000481743.2 | GATA3-204 | 975 | 143aa | ENSP00000493486.1 | 8 | 9 |
| ENST00000643001.1 | GATA3-205 | 748 | 80aa | ENSP00000494284.1 | 10 | 11 |
| ENST00000461472.1 | GATA3-203 | 895 | None | — | | |

GATA3-202 (NCBI RefSeq: NM_001002295.2)
>ENST00000379328.9 GATA3-202 cdna:protein_coding
(SEQ ID NO: 4)
GAACACTGAGCTGCCTGGCGCCGTCTTGATACTTTCAGAAAGAATGCATTCCCTGTAAAA
AAAAAAAAAAAATACTGAGAGAGGGAGAGAGAGAGAGAAGAAGAGAGAGAGACGGAGGGA
GAGCGAGACAGAGCGAGCAACGCAATCTGACCGAGCAGGTCGTACGCCGCCGCCTCCTCC
TCCTCTCTGCTCTTCGCTACCCAGGTGACCCGAGGAGGGACTCCGCCTCCGAGCGGCTGA
GGACCCCGGTGCAGAGGAGCCTGGCTCGCAGAATTGCAGAGTCGTCGCCCCTTTTTACAA
CCTGGTCCCGTTTTATTCTGCCGTACCCAGTTTTTGGATTTTTGTCTTCCCCTTCTTCTC
TTTGCTAAACGACCCCTCCAAGATAATTTTTAAAAAACCTTCTCCTTTGCTCACCTTTGC
TTCCCAGCCTTCCCATCCCCCCACCGAAAGCAAATCATTCAACGACCCCCGACCCTCCGA
CGGCAGGAGCCCCCCGACCTCCCAGGGGGACCGCCCTCCCTCCCCGCGCGCGGGTTCCGG
GCCCGGCGAGAGGGCGCGAGCACAGCCGAGGCCATGGAGGTGACGGCGGACCAGCCGCGC
TGGGTGAGCCACCACCACCCCGCCGTGCTCAACGGGCAGCACCCGGACACGCACCACCCG
GGCCTCAGCCACTCCTACATGGACGCGGCGCAGTACCCGCTGCCGGAGGAGGTGGATGTG
CTTTTTAACATCGACGGTCAAGGCAACCACGTCCCGCCCTACTACGGAAACTCGGTCAGG
GCCACGGTGCAGAGGTACCCTCCGACCCACCACGGGAGCCAGGTGTGCCGCCCGCCTCTG
CTTCATGGATCCCTACCCTGGCTGGACGGCGGCAAAGCCCTGGGCAGCCACCACACCGCC
TCCCCCTGGAATCTCAGCCCCTTCTCCAAGACGTCCATCCACCACGGCTCCCCGGGGCCC
CTCTCCGTCTACCCCCCGGCCTCGTCCTCCTCCTTGTCGGGGGGCCACGCCAGCCCGCAC
CTCTTCACCTTCCCGCCCACCCCGCCGAAGGACGTCTCCCCGGACCCATCGCTGTCCACC
CCAGGCTCGGCCGGCTCGGCCCGGCAGGACGAGAAAGAGTGCCTCAAGTACCAGGTGCCC
CTGCCCGACAGCATGAAGCTGGAGTCGTCCCACTCCCGTGGCAGCATGACCGCCCTGGGT
GGAGCCTCCTCGTCGACCCACCACCCCATCACCACCTACCCGCCCTACGTGCCCGAGTAC
AGCTCCGGACTCTTCCCCCCCAGCAGCCTGCTGGGCGGCTCCCCCACCGGCTTCGGATGC
AAGTCCAGGCCCAAGGCCCGGTCCAGCACAGAAGGCAGGGAGTGTGTGAACTGTGGGGCA
ACCTCGACCCCACTGTGGCGGCGAGATGGCACGGGACACTACCTGTGCAACGCCTGCGGG
CTCTATCACAAAATGAACGGACAGAACCGGCCCCTCATTAAGCCCAAGCGAAGGCTGTCT
GCAGCCAGGAGAGCAGGGACGTCCTGTGCGAACTGTCAGACCACCACAACCACACTCTGG
AGGAGGAATGCCAATGGGGACCCTGTCTGCAATGCCTGTGGGCTCTACTACAAGCTTCAC
AATATTAACAGACCCCTGACTATGAAGAAGGAAGGCATCCAGACCAGAAACCGAAAAATG
TCTAGCAAATCCAAAAAGTGCAAAAAAGTGCATGACTCACTGGAGGACTTCCCCAAGAAC
AGCTCGTTTAACCCGGCCGCCCTCTCCAGACACATGTCCTCCCTGAGCCACATCTCGCCC
TTCAGCCACTCCAGCCACATGCTGACCACGCCCACGCCGATGCACCCGCCATCCAGCCTG
TCCTTTGGACCACACCACCCCTCCAGCATGGTCACCGCCATGGGTTAGAGCCCTGCTCGA
TGCTCACAGGGCCCCCAGCGAGAGTCCCTGCAGTCCCTTTCGACTTGCATTTTTGCAGGA
GCAGTATCATGAAGCCTAAACGCGATGGATATATGTTTTTGAAGGCAGAAAGCAAAATTA
TGTTTGCCACTTTGCAAAGGAGCTCACTGTGGTGTCTGTGTTCCAACCACTGAATCTGGA
CCCCATCTGTGAATAAGCCATTCTGACTCATATCCCCTATTTAACAGGGTCTCTAGTGCT
GTGAAAAAAAAAATGCTGAACATTGCATATAACTTATATTGTAAGAAATACTGTACAATG
ACTTTATTGCATCTGGGTAGCTGTAAGGCATGAAGGATGCCAAGAAGTTTAAGGAATATG
GGAGAAATAGTGTGGAAATTAAGAAGAAACTAGGTCTGATATTCAAATGGACAAACTGCC
AGTTTTGTTTCCTTTCACTGGCCACAGTTGTTTGATGCATTAAAAGAAAATAAAAAAAAG
AAAAAAGAGAAAAGAAAAAAAAAGAAAAAAGTTGTAGGCGAATCATTTGTTCAAAGCTGT
TGGCCTCTGCAAAGGAAATACCAGTTCTGGGCAATCAGTGTTACCGTTCACCAGTTGCCG
TTGAGGGTTTCAGAGAGCCTTTTTCTAGGCCTACATGCTTTGTGAACAAGTCCCTGTAAT
TGTTGTTTGTATGTATAATTCAAAGCACCAAAATAAGAAAAGATGTAGATTTATTTCATC
ATATTATACAGACCGAACTGTTGTATAAATTTATTTACTGCTAGTCTTAAGAACTGCTTT
CTTTCGTTTGTTTGTTTCAATATTTTCCTTCTCTCTCAATTTTTGGTTGAATAAACTAGA
TTACATTCAGTTGGCCTAAGGTGGTTGTGCTCGGAGGGTTTCTTGTTTCTTTTCCATTTT
GTTTTTGGATGATATTTATTAAATAGCTTCTAAGAGTCCGGCGGCATCTGTCTTGTCCCT
ATTCCTGCAGCCTGTGCTGAGGGTAGCAGTGTATGAGCTACCAGCGTGCATGTCAGCGAC
CCTGGCCCGACAGGCCACGTCCTGCAATCGGCCCGGCTGCCTCTTCGCCCTGTCGTGTTC
TGTGTTAGTGATCACTGCCTTTAATACAGTCTGTTGGAATAATATTATAAGCATAATAAT
AAAGTGAAAATATTTTAAAACTA
GATA3-202 peptide: ENSP00000368632 pep:protein_coding
(SEQ ID NO: 5)
MEVTADQPRWVSHHHPAVLNGQHPDTHHPGLSHSYMDAAQYPLPEEVDVLFNIDGQGNHV
PPYYGNSVRATVQRYPPTHHGSQVCRPPLLHGSLPWLDGGKALGSHHTASPWNLSPFSKT
SIHHGSPGPLSVYPPASSSSLSGGHASPHLFTFPPTPPKDVSPDPSLSTPGSAGSARQDE
KECLKYQVPLPDSMKLESSHSRGSMTALGGASSSTHHPITTYPPYVPEYSSGLFPPSSLL
GGSPTGFGCKSRPKARSSTEGRECVNCGATSTPLWRRDGTGHYLCNACGLYHKMNGQNRP
LIKPKRRLSAARRAGTSCANCQTTTTTLWRRNANGDPVCNACGLYYKLHNINRPLTMKKE
GIQTRNRKMSSKSKKCKKVHDSLEDFPKNSSFNPAALSRHMSSISHISPFSHSSHMLTTP
TPMHPPSSLSFGPHHPSSMVTAMG
GATA3-201
ENST00000346208.4 GATA3-201 cdna:protein_coding
(SEQ ID NO: 6)
GCGAGACAGAGCGAGCAACGCAATCTGACCGAGCAGGTCGTACGCCGCCGCCTCCTCCTC
CTCTCTGCTCTTCGCTACCCAGGTGACCCGAGGAGGGACTCCGCCTCCGAGCGGCTGAGG
ACCCCGGTGCAGAGGAGCCTGGCTCGCAGAATTGCAGAGTCGTCGCCCCTTTTTACAACC
TGGTCCCGTTTTATTCTGCCGTACCCAGTTTTTGGATTTTTGTCTTCCCCTTCTTCTCTT
TGCTAAACGACCCCTCCAAGATAATTTTTAAAAAACCTTCTCCTTTGCTCACCTTTGCTT
CCCAGCCTTCCCATCCCCCCACCGAAAGCAAATCATTCAACGACCCCCGACCCTCCGACG
GCAGGAGCCCCCCGACCTCCCAGGCGGACCGCCCTCCCTCCCCGCGCGCGGGTTCCGGGC
CCGGCGAGAGGGCGCGAGCACAGCCGAGGCCATGGAGGTGACGGCGGACCAGCCGCGCTG
GGTGAGCCACCACCACCCCGCCGTGCTCAACGGGCAGCACCCGGACACGCACCACCCGGG
CCTCAGCCACTCCTACATGGACGCGGCGCAGTACCCGCTGCCGGAGGAGGTGGATGTGCT
TTTTAACATCGACGGTCAAGGCAACCACGTCCCGCCCTACTACGGAAACTCGGTCAGGGC
CACGGTGCAGAGGTACCCTCCGACCCACCACGGGAGCCAGGTGTGCCGCCCGCCTCTGCT
TCATGGATCCCTACCCTGGCTGGACGGCGGCAAAGCCCTGGGCAGCCACCACACCGCCTC
CCCCTGGAATCTCAGCCCCTTCTCCAAGACGTCCATCCACCACGGCTCCCCGGGGCCCCT
CTCCGTCTACCCCCCGGCCTCGTCCTCCTCCTTGTCGGGGGGCCACGCCAGCCCGCACCT
CTTCACCTTCCCGCCCACCCCGCCGAAGGACGTCTCCCCGGACCCATCGCTGTCCACCCC
AGGCTCGGCCGGCTCGGCCCGGCAGGACGAGAAAGAGTGCCTCAAGTACCAGGTGCCCCT
GCCCGACAGCATGAAGCTGGAGTCGTCCCACTCCCGTGGCAGCATGACCGCCCTGGGTGG
AGCCTCCTCGTCGACCCACCACCCCATCACCACCTACCCGCCCTACGTGCCCGAGTACAG
CTCCGGACTCTTCCCCCCCAGCAGCCTGCTGGGCGGCTCCCCCACCGGCTTCGGATGCAA
GTCCAGGCCCAAGGCCCGGTCCAGCACAGGCAGGGAGTGTGTGAACTGTGGGGCAACCTC
GACCCCACTGTGGCGGCGAGATGGCACGGGACACTACCTGTGCAACGCCTGCGGGCTCTA
TCACAAAATGAACGGACAGAACCGGCCCCTCATTAAGCCCAAGCGAAGGCTGTCTGCAGC
CAGGAGAGCAGGGACGTCCTGTGCGAACTGTCAGACCACCACAACCACACTCTGGAGGAG
GAATGCCAATGGGGACCCTGTCTGCAATGCCTGTGGGCTCTACTACAAGCTTCACAATAT
TAACAGACCCCTGACTATGAAGAAGGAAGGCATCCAGACCAGAAACCGAAAAATGTCTAG
CAAATCCAAAAAGTGCAAAAAAGTGCATGACTCACTGGAGGACTTCCCCAAGAACAGCTC
GTTTAACCCGGCCGCCCTCTCCAGACACATGTCCTCCCTGAGCCACATCTCGCCCTTCAG
CCACTCCAGCCACATGCTGACCACGCCCACGCCGATGCACCCGCCATCCAGCCTGTCCTT
TGGACCACACCACCCCTCCAGCATGGTCACCGCCATGGGTTAGAGCCCTGCTCGATGCTC
ACAGGGCCCCCAGCGAGAGTCCCTGCAGTCCCTTTCGACTTGCATTTTTGCAGGAGCAGT
ATCATGAAGCCTAAACGCGATGGATATATGTTTTTGAAGGCAGAAAGCAAAATTATGTTT
GCCACTTTGCAAAGGAGCTCACTGTGGTGTCTGTGTTCCAACCACTGAATCTGGACCCCA
TCTGTGAATAAGCCATTCTGACTCATATCCCCTATTTAACAGGGTCTCTAGTGCTGTGAA
AAAAAAAATGCTGAACATTGCATATAACTTATATTGTAAGAAATACTGTACAATGACTTT
ATTGCATCTGGGTAGCTGTAAGGCATGAAGGATGCCAAGAAGTTTAAGGAATATGGGAGA
AATAGTGTGGAAATTAAGAAGAAACTAGGTCTGATATTCAAATGGACAAACTGCCAGTTT
TGTTTCCTTTCACTGGCCACAGTIGTTTGATGCATTAAAAGAAAATAAAAAAAAGAAAAA
AGAGAAAAGAAAAAAAAAGAAAAAAGTTGTAGGCGAATCATTTGTTCAAAGCTGTTGGCC
TCTGCAAAGGAAATACCAGTTCTGGGCAATCAGTGTTACCGTTCACCAGTTGCCGTTGAG
GGTTTCAGAGAGCCTTTTTCTAGGCCTACATGCTTIGTGAACAAGTCCCTGTAATTGTTG
TTTGTATGTATAATTCAAAGCACCAAAATAAGAAAAGATGTAGATTTATTTCATCATATT
ATACAGACCGAACTGTTGTATAAATTTATTTACTGCTAGTCTTAAGAACTGCTTTCTTTC
GTTTGTTTGTTTCAATATTTTCCTTCTCTCTCAATTTTTGGTTGAATAAACTAGATTACA
TTCAGTTGGC
GATA3-201 peptide: ENSP00000341619 pep:protein_coding
(SEQ ID NO: 7)
MEVTADQPRWVSHHHPAVLNGQHPDTHHPGLSHSYMDAAQYPLPEEVDVLFNIDGQGNHV
PPYYGNSVRATVQRYPPTHHGSQVCRPPLLHGSLPWLDGGKALGSHHTASPWNLSPFSKT
SIHHGSPGPLSVYPPASSSSLSGGHASPHLFTFPPTPPKDVSPDPSLSTPGSAGSARQDE
KECLKYQVPLPDSMKLESSHSRGSMTALGGASSSTHHPITTYPPYVPEYSSGLFPPSSLL
GGSPTGFGCKSRPKARSSTGRECVNCGATSTPLWRRDGTGHYLCNACGLYHKMNGQNRPL
IKPKRRLSAARRAGTSCANCQTTTTTLWRRNANGDPVCNACGLYYKLHNINRPLTMKKEG
IQTRNRKMSSKSKKCKKVHDSLEDFPKNSSFNPAALSRHMSSLSHISPFSHSSHMITTPT
PMHPPSSLSFGPHHPSSMVTAMG
GATA3-204
ENST00000481743.2 GATA3-204 cdna:protein_coding
(SEQ ID NO: 8)
GGGGCGCCTCGGAGCCGCGTGCCCTCCGCCCCGGGGTGCCCATTGCGCAGAGCGTGGCCT
GGAGACCCGCGAGCCGGGGAAGGTCGCCGTGGAGTCCCGACCAGAGGCCGGGGTTGGGGT
CGGTGCAGACCGAGGGCTGGTTTCCTTGACTGTGGGAGAAACGCCGGGAGCCGGAGTGAC
CCGAGGAGGGACTCCGCCTCCGAGCGGCTGAGGACCCCGGTGCAGAGGAGCCTGGCTCGC
AGAATTGCAGAGTCGTCGCCCCTTTTTACAACCTGGTCCCGTTTTATTCTGCCGTACCCA
GTTTTTGGATTTTTGTCTTCCCCTTCTTCTCTTTGCTAAACGACCCCTCCAAGATAATTT
TTAAAAAACCTTCTCCTTTGCTCACCTTTGCTTCCCAGCCTTCCCATCCCCCCACCGAAA
GCAAATCATTCAACGACCCCCGACCCTCCGACGGCAGGAGCCCCCCGACCTCCCAGGCGG
ACCGCCCTCCCTCCCCGCGCGCGGGTTCCGGGCCCGGCGAGAGGGCGCGAGCACAGCCGA
GGCCATGGAGGTGACGGCGGACCAGCCGCGCTGGGTGAGCCACCACCACCCCGCCGTGCT
CAACGGGCAGCACCCGGACACGCACCACCCGGGCCTCAGCCACTCCTACATGGACGCGGC
GCAGTACCCGCTGCCGGAGGAGGTGGATGTGCTTTTTAACATCGACGGTCAAGGCAACCA
CGTCCCGCCCTACTACGGAAACTCGGTCAGGGCCACGGTGCAGAGGTACCCTCCGACCCA
CCACGGGAGCCAGGTGTGCCGCCCGCCTCTGCTTCATGGATCCCTACCCTGGCTGGACGG
CGGCAAAGCCCTGGGCAGCCACCACACCGCCTCCCCCTGGAATCTCAGCCCCTTCTCCAA
GACGTCCATCCACCACGGCTCCCCGGGGCCCCTCTCCGTCTACCCCCCGGCCTCGTCCTC
CTCCTTGTCGGGGGG
GATA3-204 peptide: ENSP00000493486 pep:protein_coding
(SEQ ID NO: 9)
MEVTADQPRWVSHHHPAVLNGQHPDTHHPGLSHSYMDAAQYPLPEEVDVLFNIDGQGNHV
PPYYGNSVRATVQRYPPTHHGSQVCRPPLLHGSLPWLDGGKALGSHHTASPWNLSPFSKT
SIHHGSPGPLSVYPPASSSSLSG
GATA3-205
ENST00000643001.1 GATA3-205 cdna:protein_coding
(SEQ ID NO: 10)
GCGGTGAGCAGTTCCAGCGGCCAGGACGGCAGCGGCGGCCACAGTGGCGAACTCTGCCTG
TCATTTCTGCCTTCAGATCTCCGGGCGAGTCAGGAAAAAAAATAAAAAACAGCTGGCCCT
CGGGAGCGAGCTGCCCAGGTGACCCGAGGAGGGACTCCGCCTCCGAGCGGCTGAGGACCC
CGGTGCAGAGGAGCCTGGCTCGCAGAATTGCAGAGTCGTCGCCCCTTTTTACAACCTGGT
CCCGTTTTATTCTGCCGTACCCAGTTTTTGGATTTTTGTCTTCCCCTTCTTCTCTTTGCT
AAACGACCCCTCCAAGATAATTTTTAAAAAACCTTCTCCTTTGCTCACCTTTGCTTCCCA
GCCTTCCCATCCCCCCACCGAAAGCAAATCATTCAACGACCCCCGACCCTCCGACGGCAG
GAGCCCCCCGACCTCCCAGGCGGACCGCCCTCCCTCCCCGCGCGCGGGTTCCGGGCCCGG
CGAGAGGGCGCGAGCACAGCCGAGGCCATGGAGGTGACGGGGGACCAGCCGCGCTGGGTG
AGCCACCACCACCCCGCCGTGCTCAACGGGCAGCACCCGGACACGCACCACCCGGGCCTC
AGCCACTCCTACATGGACGCGGCGCAGTACCCGCTGCCGGAGGAGGTGGATGTGCTTTTT
AACATCGACGGTCAAGGCAACCACGTCCCGCCCTACTACGGAAACTCGGTCAGGGCCACG
GTGCAGAGGTACCCTCCGACCCACCACG
>GATA3-205 peptide: ENSP00000494284 pep:protein_coding
(SEQ ID NO: 11)
MEVTADQPRWVSHHHPAVINGQHPDTHHPGLSHSYMDAAQYPLPEEVDVLFNIDGQGNHV
PPYYGNSVRATVORYPPTHH

TABLE 2
Mouse GATA3 variants
In mice, there are 6 Gata3 variants, two of which are coding.
Shown below are exemplary sequences.

| | | | | | SEQ ID | SEQ ID |
|---|---|---|---|---|---|---|
| | | | | | NO. | NO. |
| | | | | | (nucleic | (amino |
| Transcript ID | Name | bp | Protein | Translation ID | acid) | acid) |
| ENSMUST00000102976.4 | Gata3-201 | 3215 | 443aa | ENSMUSP00000100041.4 | 12 | 13 |
| ENSMUST00000130615.2 | Gata3-202 | 674 | 119aa | ENSMUSP00000119730.2 | 14 | 15 |
| ENSMUST00000153509.2 | Gata3-206 | 731 | None | — | | |
| ENSMUST00000147533.2 | Gata3-204 | 344 | None | — | | |
| ENSMUST00000142305.2 | Gata3-203 | 281 | None | — | | |
| ENSMUST00000151456.2 | Gata3-205 | 243 | None | — | | |

Gata3-201
>ENSMUST00000102976.4 Gata3-201 cdna:protein_coding
(SEQ ID NO: 12)
AGCTGTCTGCGAACACTGAGCTGCCTGGCGCCGTCTTGATAGTTTCAGAAAGAATGCATT
CCCTGTAAAAAAAAAATACTGAGAGAGGGAGAGGAGAAAGAGAGAGAGACTGAGAGAGCG
AGACATAGAGAGCTACGCAATCTGACCGGGCAGGTCACACGCCTCCTCCTCCTCCTCTAC
GCTCCTTGCTACTCAGGTGATCGGAAGAGCAACCGTCTCTGAGCGCCAAGGAATCAGTGT
GCAGTGTGGTCACACTCGGATTCCTCTCTCCCTCCTTTTTTTTTTTTTTTTTTTGACCCC
TTTATTCCTCCGTGTCTGCTTTTTTTTTGGGGGGGGGGATCGCCCTCATTCTTTTCTTTT
TCTTCTTTCCCTTCCTTTCTTTTGCTAAACTATCCCGCAAAGATTTTTCTTTCCTCCCTA
AACCCTCCTTTTTGCTCTCCTTTTCTATACCCTTAACTGCAAACAAACCATTAAACGACC
CCTCTCCTGGGCCTCCGACGGCAGGAGTCCGCGGACCTCCCAGGCCGACAGCCCTCCCTC
TACCCGCGAGGGTTCCGGGCCGGGCGAGAGGGCGCGAGCACAGCCGAGGACATGGAGGTG
ACTGCGGACCAGCCGCGCTGGGTGAGCCACCATCACCCCGCGGTCCTCAACGGTCAGCAC
CCAGACACGCACCACCCGGGCCTCGGCCATTCGTACATGGAAGCTCAGTATCCGCTGACG
GAAGAGGTGGACGTACTTTTTAACATCGATGGTCAAGGCAACCACGTCCCGTCCTACTAC
GGAAACTCCGTCAGGGCTACGGTGCAGAGGTATCCTCCGACCCACCACGGGAGCCAGGTA
TGCCGCCCGCCTCTGCTGCACGGATCTCTGCCCTGGCTGGATGGCGGCAAAGCCCTGAGC
AGCCACCACACCGCCTCGCCCTGGAACCTCAGCCCCTTCTCCAAGACGTCCATCCACCAC
GGCTCTCCGGGGCCTCTGTCCGTTTACCCTCCGGCTTCATCCTCTTCTCTGGCGGCCGGC
CACTCCAGTCCTCATCTCTTCACCTTCCCGCCCACCCCGCCGAAAGACGTCTCCCCAGAC
CCGTCGCTGTCCACCCCGGGATCCGCCGGGTCGGCCAGGCAAGATGAGAAAGAGTGCCTC
AAGTATCAGGTGCAGCTGCCAGATAGCATGAAGCTGGAGACGTCTCACTCTCGAGGCAGC
ATGACCACCCTGGGTGGGGCCTCATCCTCAGCCCACCACCCCATTACCACCTATCCGCCC
TATGTGCCCGAGTACAGCTCTGGACTCTTCCCACCCAGCAGCCTGCTGGGAGGATCCCCT
ACCGGGTTCGGATGTAAGTCGAGGCCCAAGGCACGATCCAGCACAGAAGGCAGGGAGTGT
GTGAACTGCGGGGCAACCTCTACCCCACTGTGGCGGCGAGATGGTACCGGGCACTACCTT
TGCAATGCCTGCGGACTCTACCATAAAATGAATGGGCAGAACCGGCCCCTTATCAAGCCC
AAGCGAAGGCTGTCGGCAGCAAGGAGAGCAGGGACATCCTGCGCGAACTGTCAGACCACC
ACCACCACCCTCTGGAGGAGGAACGCTAATGGGGACCCGGTCTGCAATGCCTGTGGGCTG
TACTACAAGCTTCATAATATTAACAGACCCCTGACTATGAAGAAAGAAGGCATCCAGACC
CGAAACCGGAAGATGTCTAGCAAATCGAAAAAGTGCAAAAAGGTGCATGACGCGCTGGAG
GACTTCCCCAAGAGCAGCTCCTTCAACCCGGCCGCTCTCTCCAGACACATGTCATCCCTG
AGCCACATCTCTCCCTTCAGCCACTCCAGCCACATGCTGACCACACCGACGCCCATGCAT
CCGCCCTCCGGCCTCTCCTTCGGACCTCACCACCCTTCCAGCATGGTCACCGCCATGGGT
TAGAGAGGCAGAGCCCTGCTCCACATGCGTGAGGAGTCTCCAAGTGTGCGAAGAGTTCCT
CCGACCCCTTCTACTTGCGTTTTTCGCAGGAGCAGTATCATGAAGCCCGAAAGCGACAGA
TCTGTGTTTTTGAAGGCAGAAAGCAAAATGTTTGCTTCTTTTTTCAAAGGAGCTCGAGGT
GGTGTCTGCATTCCAACCACTGAATCCGGATCCCATTTGTGAATAAGCCATTCAGACTCA
TATTCCCTATTTAACAGGGTCTCTAGTGCTGTGAAAAAAATATTGCTGAACATTGCATAT
AACTTATATTGTAAGAAATACTGTACATTTGAGGAAGACTTTATTGTACCTGGATAGCTG
TAAGAAAGGCATGAAGGACGCCAAGAGTTTTAAGGAATATAGGGGGATTAAAGTATGGAG
ATACAGAAGAAACCACTAAGTCTGATGTCCAAATGGGCACACTGTCAGTTTTGTTTCCCT
TCAGTTGTTTGATGCATTTAAAAAAAAAAAAAAGAAAGAAAAAGAAAAAAAGGGGGGGGG
GGGAGAAAAAAATAAATTAAAAAAAAAAAAAAAAAAAGAAAAGAAAGAAAAATCTAAGAA
AAAAAAAAAAAAGGTTGTAGGCAAATCATTTGTTCCAGGCTGTGAGCCTGTGCAAAAGAG
ATTTCAGATCTGGGCAATGGGTGTGTGATCTCACCCACTGAAGATCTGAGAATGTCATGG
CTAGGCCTACATGCTCTGTGAATCAGTCCCTGTAATTGTTGTTTGTATGTATAATTCAGA
AGCACCAAAATAAGAAAAGATGTAGATTTATTTCATCATATTATACAGACTGAATIGTTG
TATAAATTTATTTACTGCTAGTGTTAGGAACTGCTTTTTTTTTTTTTTTTGGITTTAATG
TTTTTTTTTTTTTTGTTTTTTGTTTTTTTTTTTCTTTCTCTCTGGATTTTTGGTTGAATA
AACTAGATTGCTTTCAGTIGACTTAAGGTGGATGTACTCTGGAGGGTTTATTTTTCCTTT
TATTATTATTTTTGATGGTATTTATTAAATAGCTTCTATGGGCCCGGGGGTACCTGTCTT
TTTCGTCACTTTTCTTGCAGCCTAAACTATGAAGGTAGCAGCGTACCAGCTACCAACATG
CATGTCAGAGACCCGGCCACTCACAGGCCTGGTCCTGAGAGCCACCTGGCTGACTGTTAG
CCCCTGTGTGTTCTGTATTAGTGATCACTGCCTTTAAACAGTCTGTTGGAATAATACTAT
AAAAATAATAATAAAGTTAAAATATTTTAAAACAA
>Gata3-201 peptide: ENSMUSP00000100041 pep:protein_coding
(SEQ ID NO:13)
MEVTADQPRWVSHHHPAVLNGQHPDTHHPGLGHSYMEAQYPLTEEVDVLFNIDGQGNHVP
SYYGNSVRATVQRYPPTHHGSQVCRPPLLHGSLPWLDGGKALSSHHTASPWNLSPFSKTS
IHHGSPGPLSVYPPASSSSLAAGHSSPHLFTFPPTPPKDVSPDPSLSTPGSAGSARQDEK
ECLKYQVQLPDSMKLETSHSRGSMTTLGGASSSAHHPITTYPPYVPEYSSGLFPPSSLLG
GSPTGFGCKSRPKARSSTEGRECVNCGATSTPLWRRDGTGHYLCNACGLYHKMNGQNRPL
IKPKRRISAARRAGTSCANCQTTTTTLWRRNANGDPVCNACGLYYKLHNINRELTMKKEG
IQTRNRKMSSKSKKCKKVHDALEDEPKSSSENPAALSRHMSSLSHISPFSHSSHMLTTPT
PMHPPSGLSFGPHHPSSMVTAMG
Gata3-202
>ENSMUST00000130615.2 Gata3-202 cdna:protein_coding
(SEQ ID NO: 14)
AGTGTTCAATCCGGAGCCGGTGTCGGCAGAAGGGAAGGAGCGCGGCGAGGCCGGCAGAGC
AGACCCGAGGAGACCTGGGGGACAGAAGGTGCTCGCGAGGGAGCTTTAGTAAGAGGAGTG
GGAGAGGGAAGAAAGTCTGCTTTGGTTCGGAAGTGCCCCTATAGGACGTACATTCTAAGG
AAAAAGAGGCTTTCAAATTGGAGGCGAACTTGTTGAAGGTGACTGCGGACCAGCCGCGCT
GGGTGAGCCACCATCACCCCGCGGTCCTCAACGGTCAGCACCCAGACACGCACCACCCGG
GCCTCGGCCATTCGTACATGGAAGCTCAGTATCCGCTGACGGAAGAGGTGGACGTACTTT
TTAACATCGATGGTCAAGGCAACCACGTCCCGTCCTACTACGGAAACTCCGTCAGGGCTA
CGGTGCAGAGGTATCCTCCGACCCACCACGGGAGCCAGGTATGCCGCCCGCCTCTGCTGC
ACGGATCTCTGCCCTGGCTGGATGGCGGCAAAGCCCTGAGCAGCCACCACACCGCCTCGC
CCTGGAACCTCAGCCCCTTCTCCAAGACGTCCATCCACCACGGCTCTCCGGGGCCTCTGT
CCGTTTACCCTCCGGCTTCATCCTCTTCTCTGGCGGCCGGCCACTCCAGTCCTCATCTCT
TCACCTTCCCGCCC
>Gata3-202 peptide: ENSMUSP00000119730 pep:protein_coding
(SEQ ID NO: 15)
MEAQYPLTEEVDVLFNIDGQGNHVPSYYGNSVRATVQRYPPTHHGSQVCRPPLLHGSLPW
LDGGKALSSHHTASPWNLSPFSKTSIHHGSPGPLSVYPPASSSSLAAGHSSPHLFTFPP

The term “GATA Binding Protein 3” and its abbreviation “GATA3” refer to a protein having the amino acid sequence of SEQ ID NO: 1, 3, 5, 7, 9, 11, 13 or 15, or a protein or polypeptide substantially homologous thereto. Such a GATA3 protein has one or more or all of the biological functions or properties of those of SEQ ID NO: 1, 3, 5, 7, 9, 11, 13, or 15, including abilities of binding to a GATA3 binding sequence, promoting chromatin opening, activating one or more GATA3 target genes, and/or recruitment of additional transcriptional regulators. Accordingly, the GATA3 protein of this disclosure can include one of SEQ ID NOs: 1, 3, 5, 7, 9, 11, 13, and 15, or a functional equivalent or fragment thereof. In general, the functional equivalent is at least 75% (e.g., any number between 75% and 100%, inclusive, e.g., 75%, 80%, 85%, 90%, 91%, 92%, 93%, 94%, 95%, 96%, 97%, 98%, and 99%) identical to one of SEQ ID NOs: 1, 3, 5, 7, 9, 11, 13, and 15. A functional equivalent of a GATA3 peptide, polypeptide, or protein refers to a peptide, polypeptide, or protein derivative of the GATA3 protein, e.g., a protein having one or more point mutations, insertions, deletions, truncations, a fusion protein, or a combination thereof. It retains substantially one or more of the activities of one of the above-mentioned GATA3 proteins.

As used herein, the term “GATA3 binding sequence” refers to a DNA sequence to which the GATA3 protein specifically binds. It may comprise a GATA3 protein binding consensus sequence such as (A/T)GATA(A/G). The GATA3 protein, as a transcription factor, affects many genes (i.e., target genes). It is known that transcription regulatory regions of genes that are transcriptionally activated by the GATA3 protein contain a GATA3 binding sequence and the binding of the GATA3 protein to the sequence is important for transcriptional activation or suppression of the genes. Examples of such target genes include, but are not limited to, the ACAN gene, the COL2A1 gene, the COL10A1 gene, the IGF2 gene, the EEF1a1 gene, the SERPINH1 gene, the IL-5 gene, the TCRα gene, and CD8. The binding of the GATA3 protein to an DNase I hypersensitive sites (DHS) sequence required for chromatin remodeling is also contributed by the binding of the GATA3 protein to the GATA3 binding sequence.

As used herein, the term “chromatin remodeling” refers to the modification of the chromatin structure in cells resulting from the binding of the GATA3 protein to a region of a chromosome.

### GAT 43 Variants

In one embodiment, the GATA3 is a human GATA3. The human GATA3 can have the amino acid sequence set out in SEQ ID NO: 1. In some embodiments, the human GATA3 has the amino acid sequence set out in SEQ ID NO: 3, 5, 7, 9, or 11. The term “GATA3” also denotes variants of the protein of SEQ ID NO: 1, 3, 5, 7, 9, 11, 13, or 15, in which one or more amino acid residues have been changed, deleted, or inserted, and which has comparable biological activity as the not modified protein.

Amino acid sequence variants of GATA3 can be prepared by introducing appropriate modifications into the nucleotide sequence encoding the GATA3, or by peptide synthesis. Such modifications include, for example, deletions from, and/or insertions into, and/or substitutions of residues within the amino acid sequences of the GATA3. Any combination of deletion, insertion and substitution can be made to arrive at the final construct, provided that the final construct possesses comparable biological activity to the human or mouse GATA3 described herein. Preferably, the modifications are conservative sequence modifications or conservative amino acid substitutions.

As used herein, the term “conservative sequence modifications” refers to amino acid modifications that do not significantly affect or alter the biological characteristics of the GATA3 described herein. Conservative amino acid substitutions are ones in which the amino acid residue is replaced With an amino acid residue having a similar side chain. Families of amino acid residues having similar side chains have been defined in the art.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties, (1) hydrophobic amino acids (norleucine, methionine, alanine, valine, leucine, and isoleucine); (2) neutral hydrophilic amino acids (cysteine, serine, threonine, asparagine, and glutamine,); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, the substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in Table 3. Amino acid substitutions may be introduced into human GATA3 and the products screened for retention of the biological activity of human GATA3.

TABLE 3

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

While many GATA3 preparations can be used, highly purified GATA3 is preferred. Examples of GATA3 and its analog include mammalian GATA3 (e.g., human or mouse GATA3) or GATA3 having substantially the same biological activity as mammalian GATA3. All naturally occurring GATA3, genetically engineered GATA3, and chemically synthesized GATA3 can be used GATA3 obtained by recombinant DNA technology may have the same amino acid sequence as naturally occurring GATA3 (SEQ ID NO: 1, 3, 5, 7, 9, 11, 13, or 15) or a functionally equivalent thereof.

The amino acid sequences of GATA3 proteins from various animal species are known. For example, as shown above, the amino acid sequences of mouse and human GATA3 protein sequences are highly conserved. The basic structure of the GATA3 protein is conserved in all species and comprises a transactivation region containing two transactivation domains, transactivation domain I, and transactivation domain II at the N-terminus. The C-terminal side of the sequence contains two zinc finger regions. The zinc finger region which is closer to N-terminus is called the N finger and the other, which is closer to C-terminus, is called the C finger. Each of these zinc finger regions contains four cysteine residues and the deletion or substitution of any one of the cysteine residues can cause a loss of function. Binding to the GATA3 binding sequences within the transcription regulatory regions of genes requires both N finger and C finger regions and the transactivation region is required for the induction of transcription activation after binding. The transactivation region is also required for GATA3 binding to nucleosome and chromatin opening.

Accordingly, the present disclosure encompasses GATA3 mutant proteins in which at least the amino acid sequence of the C or N finger region is preserved or one or more amino acids are deleted, substituted, or added so long as the ability of the protein to bind to DHS for the induction of chromatin remodeling is maintained after the deletion, substitution, or addition. Moreover, the transactivation region is important for the induction of chromatin remodeling after binding, and therefore, in some embodiments, useful GATA3 mutant proteins include this region. In other embodiments, useful GATA3 mutant proteins include all of the transactivation region and the DNA binding region.

In some embodiments, the GATA3 polypeptides described herein can comprise at least one non-naturally encoded amino acid. In some embodiments, a polypeptide comprises 1, 2, 3, 4, or more unnatural amino acids. Methods of making and introducing a non-naturally-occurring amino acid into a protein are known. See, e.g., U.S. Pat. Nos. 7,083,970; and 7,524,647. The general principles for the production of orthogonal translation systems that are suitable for making proteins that comprise one or more desired unnatural amino acids are known in the art, as are the general methods for producing orthogonal translation systems. For example, see WO 2002/086075, WO 2002/085923, WO 2004/094593, WO 2005/019415, WO 2005/007870, WO 2005/007624, WO 2006/110182, and WO 2007/103494).

### Trans-Differentiation

As disclosed herein, GATA3 was sufficient to convert non-chondrocyte cells (such as mesenchymal cells) to chondrocytes. Accordingly, this disclosure provides agents that can convert non-chondrocyte cells (i.e., starting cells) to chondrocyte cells, thereby supplying an unlimited cell source for modeling and understanding chondrocyte or joint diseases, testing drug efficacy and toxicity and cell replacement therapy.

### Starting Cells

Various cells from a subject or animal can be used as the starting cells. For example, somatic cells can be used as the starting cells. As used herein, the term “somatic cell” refers to any cell other than a germ cell, a cell present in or obtained from a pre-implantation embryo, or a cell resulting from the proliferation of such a cell in vitro. Stated another way, a somatic cell refers to any cell forming the body of an organism, as opposed to a germline cell. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated, pluripotent, embryonic stem cells—is a somatic cell: internal organs, skin, bones, blood, and connective tissue are all made up of somatic cells.

In some embodiments, the somatic cell is a “non-embryonic somatic cell,” which means a somatic cell that is not present in or obtained from an embryo and does not result from the proliferation of such a cell in vitro. In some embodiments, the somatic cell is an “adult somatic cell,” which means a cell that is present in or obtained from an organism other than an embryo or a fetus or results from the proliferation of such a cell in vitro. Unless otherwise indicated, the compositions and methods for reprogramming a somatic cell described herein can be performed both in vivo and in vitro (where in vivo is practiced when a somatic cell is present within a subject, and where in vitro is practiced using an isolated somatic cell maintained in culture). The term excludes gametes, germ cells, gametocytes, fertilized eggs, or embryos at development stages before the blastula stage.

In some embodiments, the starting cells are stem cells. The stem cells that are useful for the methods described herein include, but not limited to, embryonic stem cells, induced pluripotent stem (iPS) cells, mesenchymal stem cells, bone-marrow derived stem cells, hematopoietic stem cells, chondrocyte progenitor cells, epidermal stem cells, gastrointestinal stem cells, neural stem cells, hepatic stem cells, adipose-derived mesenchymal stem cells, pancreatic progenitor cells, hair follicular stem cells, endothelial progenitor cells, and smooth muscle progenitor cells. The stem cells can be pluripotent or multipotent. In some embodiments, the stem cell is an adult, fetal or embryonic stem cell. The stem cells can be isolated from the umbilical, placenta, amniotic fluid, chorion villi, blastocysts, bone marrow, adipose tissue, brain, peripheral blood, blood vessels, skeletal muscle, and skin.

In some embodiments, the starting cells are differentiated cells. Examples include a fibroblast, an epithelium cell, an endothelial cell, a mesenchymal cell, a blood cell, a neuron, an embryonic cell, or a cell derived from a tissue or organ of a subject. These differentiated cells differ from stem cells in that differentiated cells generally do not undergo self-renewing proliferation while stem cells can undergo self-renewing cell division to give rise to phenotypically and genotypically identical daughters for an indefinite time and ultimately can differentiate into at least one final cell type.

In some embodiments, the starting cells are autologous, isogeneic, allogeneic, xenogeneic cells, or any combination thereof. Autologous cells are isolated from the same individual to which they will be re-implanted. Allogeneic cells are isolated from a donor of the same species Xenogeneic cells are isolated from a donor of another species. Syngeneic or isogeneic cells are isolated from genetically identical organisms, such as twins, clones, or highly inbred research animal models.

Any type of the stating cells can be differentiated, reprogrammed, or trans-differentiated into chondrocytes or chondrogenic cells by increasing the expression level or activity level of GATA3 in the cells. For example, a GATA3 polypeptide, a nucleic acid encoding the GATA3 polypeptide, a GATA3 analog, or a GATA3 activator can be used as agents to increase the expression or activity level and thereby reprogram non-chondrocyte cells to become chondrocytes and form cartilages. Therefore, these agents can be applied as therapeutic agents for cell-based therapy in diseases such as arthritis, TMD, and numerous congenital and cartilage degenerative diseases. For instance, patient-specific chondrocytes can be derived from non-chondrocyte cells, such as iPSC, or directly induced somatic cell types, becoming inducible chondrocytes (iCHON) for personalized regenerative medicine. A number of cell types have been indicated to be reprogrammable to become induced Pluripotent Cells, e.g., Hepatocyte, Neuron. Haematopoietic progenitor, Muscle, and Cardiomyocyte. However, there is currently no report or method described for the induction of chondrocytes (Balsalobre and Drouin, Nature Reviews Molecular and Cell Biology, 2022). The discoveries described herein allow one to utilize the chondrocyte inducible agents, including GATA3, for cartilage regeneration to benefit 58.5 million people (24% of adults) suffering from arthritis and 10 million suffering from TMD in the US.

Examples of the chondrocyte inducible agents or therapeutic agents can include GATA3 activators, GATA3 proteins, GATA3 analogs, GATA3 isoforms, GATA3 mimetics, GATA3 fragments, hybrid GATA3 proteins, fusion proteins oligomers and multimers of the above, homologs of the above, including post-translation modification (e.g., glycosylation pattern) variants of the above, and mutants of the above, regardless of the method of synthesis or manufacture thereof including but not limited to, recombinant vector expression whether produced from cDNA or genomic DNA, synthetic, transgenic and gene activated methods.

As used herein, a GATA3 activator, a GATA3 positive regulator, a GATA3 booster, a GATA3 expression enhancer, or GATA3 agonist refers to a molecule or a composition or a stimulation that can increase the gene expression level, the mRNA level, the protein level, or the activity of GATA3 directly or indirectly.

### Forced Expression

In some embodiments, GATA3 is forced expressed or overexpressed in a non-chondrocyte. For expressing GATA3, the disclosure provides a nucleic acid that encodes any of the GATA3 polypeptides and variants described herein. Preferably, the nucleotide sequences are isolated and/or purified.

The present disclosure also provides recombinant expression cassettes or genetic constructs having one or more of the nucleotide sequences described herein. Examples of the cassettes or constructs can be included in a vector, such as a plasmid or viral vector, into which a nucleic acid sequence encoding GATA3 has been inserted, in a forward or reverse orientation. In a preferred embodiment, the cassette or construct further includes regulatory sequences, including a promoter, operably linked to the sequence. Large numbers of suitable vectors and promoters are known to those of skill in the art and are commercially available. Appropriate cloning and expression vectors for use with prokaryotic and eukaryotic hosts are also described in Sambrook et al. (2012, Molecular Cloning: A Laboratory Manual, Cold Spring Harbor Press).

The vector can be an expression vector. Examples of expression vectors include chromosomal, nonchromosomal, and synthetic nucleic acid sequences, e.g., Simian virus 40 (SV40), bacterial plasmids, phage DNA, baculovirus, yeast plasmids, vectors derived from combinations of plasmids and phage DNA, viral nucleic acid such as vaccinia, retroviruses, adenovirus, fowl pox virus, and pseudorabies. However, any other vector may be used as long as it is replicable and viable in the host. The appropriate nucleic acid sequence may be inserted into the vector by a variety of procedures in general, a nucleic acid sequence encoding one of the GATA3 polypeptides or variants described above can be inserted into an appropriate restriction endonuclease site(s) by procedures known in the art. Such procedures and related sub-cloning procedures are within the scope of those skilled in the art.

The nucleic acid sequence in the aforementioned expression vector is preferably operatively linked to an appropriate transcription control sequence (promoter) to direct mRNA synthesis. Examples of such promoters include the retroviral long terminal (LTR) or SV40 promoter, the E. coli lac or trp promoter, the phage lambda PL promoter, and other promoters known to control the expression of genes in prokaryotic or eukaryotic cells or viruses. The expression vector can also contain a ribosome binding site for translation initiation and a transcription terminator. The vector may include appropriate sequences for amplifying expression. In addition, the expression vector preferably contains one or more selectable marker genes to provide a phenotypic trait for the selection of transformed host cells such as dihydrofolate reductase or neomycin resistance for eukaryotic cell cultures, or as tetracycline or ampicillin resistance in E. coli.

The vector containing the appropriate nucleic acid sequences as described above, as well as an appropriate promoter or control sequence, can be employed to transform, transfect, or infect an appropriate host to permit the host to express the polypeptides described above. Examples of suitable expression hosts include bacterial cells (e.g. E. coli, Streptomyces, Salmonella typhimurium), fungal cells (yeast), insect cells (e.g., Drosophila and Spodoptera frugiperda (Sf9)), animal cells (e.g., CHO, COS, and HEK 293), adenoviruses, and plant cells. The selection of an appropriate host is within the scope of those skilled in the art. In some embodiments, the present disclosure provides methods for producing the above mentioned polypeptides by transfecting a host cell with an expression vector having a nucleotide sequence that encodes one of the polypeptides. The host cells are then cultured under a suitable condition, which allows for the expression of the polypeptide.

The above-discussed nucleic acids encoding one or more of the polypeptides mentioned above can be cloned in a vector for delivering to cells in vitro or in vivo. For in vivo uses, the delivery can target a specific tissue or organ (e.g., joint). Targeted delivery involves the use of vectors (e.g., organ-homing peptides) that are targeted to specific organs or tissues after local or systemic administration.

In certain embodiments, the present disclosure provides methods for in vivo production of the above-mentioned chondrocytes or chondrogenic cells. Such a method would achieve its therapeutic effect by introduction of the nucleic acid sequences into cells or tissues of a human or a non-human animal in need of an increase of cartilage. Delivery of the nucleic acid sequences can be achieved using a recombinant expression vector such as a chimeric virus or a colloidal dispersion system. One of the preferred therapeutic deliveries of the nucleic acid sequences is the use of viral vectors.

### 1. Viral Vectors

In some embodiments, a nucleic acid encoding GATA3 can be inserted into, or encoded by, vectors such as viral vectors. Various viral vectors which can be utilized for gene therapy disclosed herein include adenovirus, herpes virus, vaccinia, or, an RNA virus such as a retrovirus and a lentivirus. Preferably, the viral vectors may be Herpesvirus (HSV) vectors, retroviral vectors, adenoviral vectors, adeno-associated virus (AAV) vectors, lentiviral vectors, and the like.

i. Retroviral Vectors

In some embodiments, the GATA3 protein may be encoded by a retroviral vector Preferably, the retroviral vector is a lentivirus or a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. See, e.g., U.S. Pat. Nos. 5,399,346; 5,124,263; 4,650,764 and 4,980,289; the content of each of which is incorporated herein by reference in their entirety.

Lentiviruses, such as HIV, are “slow viruses.” Vectors derived from lentiviruses can be expressed long-term in the host cells after a few administrations to the patients, e.g., via ex vivo transduced stem cells or progenitor cells. For most diseases and disorders, including genetic diseases, cancer, and neurological disease, long-term expression is crucial to successful treatment. Regarding safety with lentiviral vectors, a number of strategies for eliminating the ability of lentiviral vectors to replicate have now been known in the art. See e.g., US 20210401868 and 20210403517, each of which is incorporated herein by reference in its entirety. For example, the deletion of promoter and enhancer elements from the U3 region of the long terminal repeat (LTR) is thought to have no LTR-directed transcription. The resulting vectors are called “self-inactivating” (SIN).

Lentiviral vectors are particularly suitable for achieving long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity. In general, a suitable vector contains an origin of replication functional in at least one organism, a promoter sequence, convenient restriction endonuclease sites, and one or more selectable markers. (e.g. WO01/96584 and WO01/29058; and U.S. Pat. No. 6,326,193). Several vector promoter sequences are available for the expression of the transgenes. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is EF1a. However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter. MoMuLV promoter, an avian leukemia virus promoter, an Epstein-Barr virus immediate early promoter, a Rous sarcoma virus promoter, as well as human gene promoters such as but not limited to, the actin promoter, the myosin promoter, the hemoglobin promoter, and the creatine kinase promoter. Inducible promoters include, but are not limited to a metallothionein promoter, a glucocorticoid promoter, a progesterone promoter, and a tetracycline promoter.

The present disclosure provides a recombinant lentivirus capable of infecting dividing and non-dividing cells. The virus is useful for the in vivo and ex vivo transfer and expression of nucleic acid sequences. Lentiviral vectors of the present disclosure may be lentiviral transfer plasmids or infectious lentiviral particles. Construction of lentiviral vectors, helper constructs, envelope constructs, etc., for use in lentiviral transfer systems, has been described in, e.g., US 20210401868 and 20210403517, each of which is incorporated herein by reference in its entirety.

ii. Adenoviruses

Adenoviruses are eukaryotic DNA viruses that can be modified to efficiently deliver nucleic acid to a variety of cell types in vivo and have been used extensively in gene therapy protocols. Various replication defective adenovirus and minimum adenovirus vectors have been described for nucleic acid therapeutics. See, e.g., PCT Patent Publication Nos. WO199426914, WO 199502697, WO199428152. WO199412649, WO199502697, and WO199622378; the content of each of which is incorporated by reference in their entirety.

Such adenoviral vectors may also be used to deliver therapeutic molecules of the present disclosure to cells.

iii. Adeno-Associated Virus

In some embodiments, the viral vectors are adeno-associated virus (AAV) vectors. AAVs are parvoviruses with a linear single-stranded DNA genome and variants thereof. The term AAV as used herein covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome. Site-specific integration, as opposed to random integration, is believed to likely result in a predictable long-term expression profile. The insertion site of AAV into the human genome is referred to as AAVS1. Once introduced into a cell, RNAs or polypeptides encoded by the nucleic acid can be expressed by the cell. Because AAV is not associated with any pathogenic disease in humans, a nucleic acid delivered by AAV can be used to express a therapeutic RNA or polypeptide for the treatment of a disease, disorder, and/or condition in a human subject.

The adeno-associated virus is a widely used gene therapy vector due to its clinical safety record, non-pathogenic nature, ability to infect non-dividing cells, and ability to provide long-term gene expression after a single administration. Currently, many human and non-human primate AAV serotypes have been identified. AAV vectors have demonstrated safety in hundreds of clinical trials worldwide, and clinical efficacy has been shown in trials of hemophilia B, spinal muscular atrophy, alpha 1 antitrypsin, and Leber congenital amaurosis.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. Examples include AAV1, AAV2, AAV, AAV3 (including AAV3A and AAV3B), AAV4. AAV5, AAV6, AAV7. AAV8, AAV9, AAV10, AAV12, AAVrh10. AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions, and substitutions, etc.), such as variants referred to as AAV2i8, NP4, NP22, NP66, DJ, DJ/8, DJ/9, LK3, RHM4-1, among many others.

All of the vectors discussed above can transfer or incorporate a gene for a selectable marker so that transduced cells can be identified and generated. Viral vectors can be made target-specific by attaching, for example, a sugar, a glycolipid, or a protein. Preferred targeting can be accomplished by using a target-specific antibody or hormone that has a receptor in the target. Those of skill in the art will recognize that specific polynucleotide sequences can be inserted into the viral genome or attached to a viral envelope to allow target-specific delivery of the viral vector.

### 2. Non-Viral Deliveries

In addition to virus-based delivery, nucleic acids, genetic constructs, expression cassettes, vectors, and even proteins can be introduced into a target cell via non-viral means.

One example is a colloidal dispersion system. Colloidal dispersion systems include macromolecule complexes, nanocapsules, microspheres, beads, and lipid-based systems including oil-in-water emulsions, micelles, mixed micelles, and liposomes. A preferred colloidal system is a liposome. Liposomes are artificial membrane vesicles that are useful as delivery vehicles in vitro and in vivo. RNA, DNA, and intact virions can be encapsulated within the aqueous interior and delivered to cells in a biologically active form Methods for efficient gene transfer using a liposome vehicle are known in the art. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Exemplary phospholipids include egg phosphatidylcholine, dipalmitoylphosphatidylcholine, and distearoyl-phosphatidylcholine. The targeting of liposomes is also possible based on, for example, tissue-specificity, cell-specificity, and organelle-specificity and is known in the art.

When used in vivo, it is desirable to use a reversible delivery-expression system. To that end, the Cre-loxP or FLP/FRT system and other similar systems can be used for the reversible delivery expression of one or more of the above-described nucleic acids. See WO2005/112620, WO2005/039643, U.S. Applications 20050130919, 20030022375, 20020022018, 20030027335, and 20040216178. In particular, the reversible delivery-expression system described in US Application NO 20100284990 can be used to provide a selective or emergency shut-off.

### Protein Transfection

A GATA3 polypeptide described herein can be induced into cells of interest via protein transfection or transduction. The polypeptide can be obtained as a recombinant polypeptide. For example, to prepare a recombinant polypeptide, a nucleic acid encoding it can be linked to another nucleic acid encoding a fusion partner, e.g., glutathione-s-transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention. Alternatively, the peptides/polypeptides/proteins of the invention can be chemically synthesized (see e.g., Creighton, “Proteins: Structures and Molecular Principles.” W.H. Freeman & Co., NY, 1983). For additional guidance, skilled artisans may consult Ausubel et al. (Current Protocols in Molecular Biology and Short Protocols in Molecular Biology, 3rd Ed. 2002 & 2002), Sambrook et al. (Molecular Cloning. A Laboratory Manual, Cold Spring Harbor Press, Cold Spring Harbor, NY, 2012), and chemical synthesis Gait, M. J. Ed. (Oligonucleotide Synthesis, IRL Press, Oxford, 1984).

Due to its functions as a transcription factor, the above-disclosed GATA3 polypeptide can be associated with, e.g., conjugated or fused to, one or more of an amino acid sequence comprising a nuclear localization signal (NLS), a cell-penetrating peptide (CPP) sequence, and the like. In this manner, a composition of this disclosure as discussed below can include a transport enhancer. For example, the composition may include a penetration enhancing agent, such as MSM, for the delivery of the transcription factor or related therapeutic polypeptide to a cell and/or through the cell membrane and into the nucleus of the cell. The transcription factor then functions, e.g., to regulate chromatin opening and/or transcription of target genes, thereby resulting in an induction of chondrocytes or chondrogenic cell fate/lineage. The GATA3 polypeptide may be delivered by itself or as a fusion with one or more of an NLS. CPP, and/or other domains. See, e.g., Tachikawa et al. PNAS (2004) vol. 101, no. 42:15225-15230, US 20090156503.

A cell-penetrating peptide (CPP) generally consists of less than 30 amino acids and has a net positive charge. CPPs internalize in living animal cells in vitro and in vivo in an endocytotic or receptor/energy-independent manner. There are several classes of CPPs with various origins, from totally protein-derived CPPs via chimeric CPPs to completely synthetic CPPs. Examples of CPPs are known in the art. See, e.g., U.S. Application Nos. 20090099066 and 20100279918. It is known that CPPs can deliver an exogenous protein to various cells.

Although the above-described factors to be delivered to a cell may be fusion proteins including an NLS and/or CPP, in certain instances, the protein does not include an NLS and/or a CPP as the transport enhancer may serve the function of delivering the biologically active agent directly to the cell, and/or through the cell membrane into the cytoplasm of the cell and/or into the nucleus of the cell as desired. For instance, in certain instances, it may be desirable to deliver a biologically active protein to the cell wherein the protein is not conjugated or fused to another molecule. In such an instance, any biologically active protein may be delivered directly in conjunction with the transport enhancer.

Various other cell-penetrating molecules are known in the art and can be used in this invention. In some embodiments, a cell-penetrating molecule may comprise a phosphorothioate nucleic acid. It is known in the art that phosphorothioate nucleic acids can enhance the intracellular delivery of both proteins and nucleic acids. See, e.g., US20190365905. WO2019014648, US 20190119259, US 20180243436, US 20180230237, US 20180008667, US 20160317671, and Herrmann et al, JCI Insight. 2019.

The term “phosphorothioate nucleic acid” refers to a nucleic acid in which one or more internucleotide linkages are through a phosphorothioate moiety (thiophosphate). The phosphorothioate moiety may be a monothiophosphate (—P(O)₃(S)³⁻—) or a dithiophosphate (—P(O)₂(S)₂³⁻—). In some embodiments, the phosphorothioate nucleic acid can be a monothiophosphate nucleic acid. In some embodiments, one or more of the nucleosides of a phosphorothioate nucleic acid can be linked through a phosphorothioate moiety (e.g., monothiophosphate), and the remaining nucleosides can be linked through a phosphodiester moiety (—P(O)₄³⁻—). In some embodiments, one or more of the nucleosides of a phosphorothioate nucleic acid can be linked through a phosphorothioate moiety (e.g., monothiophosphate), and the remaining nucleosides can be linked through a methylphosphonate linkage. In some embodiments, all the nucleosides of a phosphorothioate nucleic acid can be linked through a phosphorothioate moiety (e.g., a monothiophosphate).

Phosphorothioate oligonucleotides (phosphorothioate nucleic acids) are typically from about 5, 6, 7, 8, 9, 10, 12, 15, 20, 25, 30, 40, 50 or more nucleotides in length up to about 100 nucleotides in length Phosphorothioate nucleic acids may also be longer in lengths, e.g., 200, 300, 500, 1000, 2000, 3000, 5000, 7000, 10,000, etc. As described above, in certain embodiments, the phosphorothioate nucleic acids herein contain one or more phosphodiester bonds. In other embodiments, the phosphorothioate nucleic acids can include alternate backbones (e.g., mimics or analogs of phosphodiesters as known in the art, such as boranophosphate, methylphosphonate, phosphoramidate, or O-methylphosphoroamidite linkages (see Eckstein, Oligonucleotides and Analogues: A Practical Approach, Oxford University Press). The phosphorothioate nucleic acids may also include one or more nucleic acid analog monomers known in the art, such as peptide nucleic acid monomer or polymer, locked nucleic acid monomer or polymer, morpholino monomer or polymer, glycol nucleic acid monomer or polymer, or threose nucleic acid monomer or polymer. Other analog nucleic acids include those with positive backbones; non-ionic backbones, and non-ribose backbones, including those described in U S. Pat. Nos. 5,235,033 and 5,034,506, and Chapters 6 and 7, ASC Symposium Series 580, Carbohydrate Modifications in Antisense Research, Sanghui & Cook, eds.

Both naturally occurring GATA3 proteins, as well as recombinant GATA3 proteins, can be used in the methods and applications disclosed herein. The term “GATA3” or “GATA Binding Protein 3” also covers chemically modified GATA3. Examples of chemically modified GATA3 include GATA3 subjected to a conformational change, addition or deletion of one or more post-translation modifications and GATA3 to which a compound such as a phosphorothioate nucleic acids or a polyethylene glycol has been bound. Once purified and tested by standard methods or according to the method described in the examples below, GATA3 can be included in a pharmaceutical composition.

Recombinant GATA3 protein may be prepared via expression in eukaryotic cells, for example in CHO cells, BHK cells, or HeLa cells by recombinant DNA technology or by endogenous gene activation, i.e., the GATA3 protein is expressed by endogenous gene activation, see, for example, U.S. Pat. Nos. 5,733,761, 5,641,670, 5,733,746, WO 93/09222, WO 94/12650, WO 95/31560, WO 90/11354, WO 01/06667, and WO 91/09955. These documents are incorporated herein by reference.

### Cartilage Regeneration Products

The chondrocytes or chondrogenic cells described herein can be provided in combination with other types of cells, agents, materials, and structures for various uses, such as tissue engineering and treatment of any cartilage, joint, or bone with a defect. Accordingly, this application provides a cartilage regeneration product or cartilage regeneration formulation comprising at least one chondrocyte or chondrogenic cell and optionally at least one of such other types of cells, agents, material, and structure. To that end, the cells can be in three-dimensional (3D) synthetic, semi-synthetic, or living biological tissues.

In one example, the cartilage regeneration product/formulation comprises at least one chondrocyte or chondrogenic cell and at least one scaffold suitable for carrying the cell. The scaffold may comprise any 3D-printed scaffold suitable for carrying the cell. The cartilage regeneration product/formulation is suitable for cartilage regeneration, joint regeneration, or a combination thereof. The cartilage regeneration product/formulation may further comprise a growth factor as mentioned above. The scaffold can comprise various suitable materials, such as hydroxyapatite (HA) tricalcium phosphate (TCP), and a polymer. The polymer may be prepared by using photocurable polymers and/or monomers.

The scaffold may comprise a porous, 3D network of interconnected void spaces. The scaffold may be any scaffold suitable to incorporate the cells and/or growth factors disclosed herein to aid in forming a direct contact and/or an indirect contact of these cells and/or growth factors with a tissue (e.g., cartilage) for the regeneration of this tissue (e.g., cartilage). The scaffold may incorporate the cells and/or growth factors in any form, for example, by carrying, by supporting, by adsorbing, by absorbing, by encapsulating, by holding, and/or by adhering to the cells and/or growth factors. The scaffold may have any shape or geometry. The scaffold may have any pore size. The scaffold may have any porosity (i.e., void volume.) The scaffold may have any form. The scaffold may have any mechanical strength.

Cartilage defects may form in different parts of an animal or a human body. These defects may have any shape and size. Scaffolds suitable for the treatment of such defects may have shapes, volumes, and sizes that can, for example, fit to or resemble the defect shape and size Such scaffolds may also have pore volumes, pore sizes, and/or pore shapes that resemble the cartilage for which the cartilage regeneration products that comprise such scaffolds are designed for their treatment. Such scaffolds may also have pores with pore sizes sufficiently small such that these scaffolds can contain the cells described herein within their porous structures and allow the cartilage regeneration product to be implanted and the treatment can be successfully carried out. The cartilage regeneration products/formulation can have a mechanical strength sufficient enough to handle load bearing conditions of their implantation to a body. It can also have a mechanical strength sufficient enough to handle load bearing conditions of cartilage or bones during motion (e.g., walking) and/or weight of the bodies.

The scaffold may comprise any material. For example, the scaffold may comprise a non-resorbable material, resorbable material, or a mixture thereof. The resorbable material may be resorbed by the body of a patient and eventually replaced with healthy tissue. A “resorbable” material may comprise, for example, a biocompatible, bioabsorbable, biodegradable polymer, any similar material, or a mixture thereof.

A biocompatible material is a material that may be accepted by and to the function of a body of a patient without causing a significant foreign body response (such as, for example, an immune, inflammatory, thrombogenic, or like a response), and/or is a material that may not be clinically contraindicated for administration into a tissue or organ. The biodegradable material may comprise a material that is absorbable or degradable when administered in vivo and/or under in vitro conditions. Biodegradation may occur through the action of biological agents, either directly or indirectly.

The scaffold may comprise a solid, a liquid, or a mixture thereof. For example, the scaffold may be a paste. For example, the scaffold may comprise a paste comprising a mixture of hydroxyapatite and tricalcium phosphate (HA/TCP). This scaffold, for example, may be prepared by mixing hydroxyapatite (HA) and tricalcium phosphate (TCP) with a formulation comprising a liquid to prepare a paste. For example, the chondrogenic cell/chondrocyte formulation may comprise a liquid; and mixing of such cell formulation with hydroxyapatite (HA) and tricalcium phosphate (TCP) may form a paste. This type of scaffold is called an HA/TCP scaffold herein. A mixture comprising HA and TCP (HA/TCP) may be formed from equal amounts of HA and TCP in weight, for example, 50 wt % HA and 50 wt % TCP, unless otherwise stated. However, the mixture comprising HA and TCP may have any composition, for example, varying in the range of 0 wt % HA to 100 wt % TCP. For example, an HA concentration higher than 10 wt %, higher than 20 wt %, higher than 30 wt %, higher than 40 wt %, higher than 50 wt %, higher than 60 wt %, higher than 70 wt %, higher than 80 wt %, or higher than 90 wt % is within the scope of this application.

The scaffolds described herein may comprise any biodegradable polymer. For example, the scaffold may comprise a synthetic polymer, naturally occurring polymer, or a mixture thereof. Examples of suitable biodegradable polymers may be polylactide (PLA), polyglycolide (PGA), poly(lactide-co-glycolide) (PLGA), poly-E-caprolactone, polydioxanone trimethylene carbonate, polyhybroxyalkonates (e.g., poly(hydroxybutyrate)), poly(ethyl glutamate), poly(DTH iminocarbonyl (bisphenol A iminocarbonate), poly(ortho ester), polycyanoacrylates, fibrin, casein, serum albumin, collagen, gelatin, lecithin, chitosan, alginate, poly-amino acids (such as poly lysine), and a mixture thereof.

The scaffold may further comprise one or more of the growth factors described above, the growth factor may be continuously released to the surrounding issue after the cartilage regeneration product is implanted into the defect site. The release rate of the growth factor may be controlled through the scaffolds' chemical composition and/or pore structure.

The scaffold may be manufactured by any technique. For example, the scaffold may be manufactured by hand, and/or by using a machine. For example, the scaffold may be manufactured by additive manufacturing and/or manufacturing. For example, the scaffold may be manufactured using a combination of more than one such manufacturing technique.

In one embodiment, the cells are used in a “bio-printing” process to generate a spatially-controlled cell pattern using a 3D printing technology. Any bio-printing or bio-fabricating process known in the art can be used, e.g., as described in U.S. Pat. App. Pub. Nos. 20140099709, 20140093932, 20140274802, 20140012407, 20130345794, 20130190210 and 20130164339; and U.S. Pat. No. 8,691,974.

For example, in one embodiment, a printer cartridge is filled with a suspension of suitable cells and a gel. The alternating patterns of the gel and cells can be printed using a standard print nozzle. In an alternative embodiment, a NOVOGEN (San Diego, Calif.) MMX™, or Organovo Holdings, Inc., bioprinters can be used for 3D bioprinting. These and equivalent “bio-printers” can be optimized to “print”, or fabricate, cartilage tissue, bone tissue, and other tissues, all of which are suitable for surgical therapy and transplantation.

Any 3D printing technique may be used to manufacture the scaffold. The 3D printing technique or additive manufacturing (AM) may be a process for making a physical object from a 3D digital model, typically by laying down many successive thin layers of material. Such thin layers of material may be formed under computer control. Examples of 3D printing technologies may be Stereolithography (SLA), Digital Light Processing (DLP), Fused deposition modeling (FDM), Selective Laser Sintering (SLS), Selective laser melting (SLM), Electronic Beam Melting (EBM), Laminated object manufacturing (LOM), Binder jetting (BJ), Material Jetting (MJ) or Wax Casting (WC), or a combination thereof.

The scaffold, including the 3D printed scaffold, for example, may be manufactured by using a formulation comprising polycaprolactone dimethacrylate (PCLDA), calcium phosphate, HA, TCP, polyethylene glycol diacrylate (PEGDA), gelatin methacryloyl (GeIMA), or a mixture thereof.

This cartilage regeneration product/formulation can be used in regenerating cartilage. Such a cartilage regeneration method may comprise implanting the cartilage regeneration product/formulation in or across cartilage or joint defect. This defect may be any defect, such as that in osteoarthritis. The size of the defect may be any size. For example, the size of this cartilage or joint defect may be a critical size. The cartilage or joint defect may be formed due to a congenital malformation. The congenital defect may be a defect related to a cleft lip and/or a cleft palate. The defect may be formed because of surgery, accident, and/or disease. This defect may be formed as a result of surgery carried out to treat craniosynostosis.

### Pharmaceutical Composition

The present application further provides a composition comprising (i) a carrier and (ii) one or two or more of the following: a GATA3 activator, a genetic construct comprising a nucleic acid sequence encoding GATA3, a GATA3 polypeptide, a cell obtained according to methods described herein or cells derived therefrom (such as progeny cells), the cartilage regeneration product or artificial cartilage described herein. The composition can be a pharmaceutical composition where the carrier is pharmaceutically acceptable.

A composition for pharmaceutical use, e.g., a scaffold or implant with cells and/or factors, can include, depending on the formulation desired, pharmaceutically acceptable, non-toxic carriers of diluents, which are defined as vehicles commonly used to formulate pharmaceutical compositions for animal or human administration. The diluent can be selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients, and the like. The compositions can also include additional substances to approximate physiological conditions, such as pH adjusting and buffering agents, toxicity adjusting agents, wetting agents, and detergents.

The composition can also include any of a variety of stabilizing agents, such as an antioxidant for example. When the pharmaceutical composition includes a polypeptide (e.g., a GATA3 protein), the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake by a target cell). Examples of such modifications or complexing agents include sulfate, gluconate, citrate, and phosphate. The polypeptides of a composition can also be complexed with molecules that enhance their in vivo attributes. Such molecules include, for example, lipids, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences. Mace Publishing Company, Philadelphia. Pa., 17th ed. (1985). For a brief review of methods for drug delivery, see Langer, Science 249:1527-1533 (1990).

The pharmaceutical composition described herein (e.g., cells, polypeptides, nucleic acids, activators, vectors, or products) alone or in combinations with various factors, can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

Data obtained from cell culture and/or animal studies can be used in formulating a range of dosages for humans. The dosage can vary within this range depending upon the dosage form employed and the route of administration utilized.

The components used to formulate the pharmaceutical compositions are preferable of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic, and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression of the disease condition as required. Utilizing animal data, and other information available for the agent, a clinician can determine the maximum safe dose for an individual, depending on the route of administration. For instance, an intravenously administered dose may be more than a locally administered dose, given the greater body of fluid into which the therapeutic composition is being administered. Similarly, compositions that are rapidly cleared from the body may be administered at higher doses, or in repeated doses, in order to maintain a therapeutic concentration. Utilizing ordinary skills, the competent clinician will be able to optimize the dosage of a particular therapeutic in the course of routine clinical trials.

Mammalian species that may be treated with the present methods include canines and felines, equines, bovines; ovines; etc. and primates, particularly humans. Animal models, particularly small mammals, e.g., murine, lagomorpha, etc. may be used for experimental investigations.

### Uses

The cells, polypeptides, nucleic acids, vectors, activators, compositions, formulations, and products disclosed herein can be used for various purposes including treating related disorders and in tissue engineering.

In some embodiments, the cells, polypeptides, nucleic acids, vectors, activators, compositions, formulations, and products disclosed herein can be used in the treatment of a subject, such as a human patient, in need of cartilage replacement therapy. Examples of such subjects can be subjects suffering from conditions associated with the loss of cartilage from osteoarthritis, TMD, genetic defects, diseases, etc. Patients having diseases and disorders characterized by such conditions w ill benefit greatly from a treatment protocol of the pending claimed invention.

An effective amount of the pharmaceutical composition is the amount that will result in an increase in the number of chondrocytes or cartilage at the site of implant, and/or will result in a measurable reduction in the rate of disease progression in vivo. For example, an effective amount of a pharmaceutical composition will increase cartilage mass by at least about 5%, at least about 10%, at least about 20%, preferably from about 20% to about 50%, and even more preferably, by greater than 50% (e.g., from about 50% to about 100%) as compared to the appropriate control, the control typically being a subject not treated with the composition.

The methods described above can be used in combined therapies with, e.g., therapies that are already known in the art to provide relief from symptoms associated with the aforementioned diseases, disorders, and conditions. The combined use of a pharmaceutical composition described herein and these other agents may have the advantages that the required dosages for the individual drugs are lower, and the effect of the different drugs can be complementary.

In some embodiments, an effective dose of the cells described herein, preferably chondrocytes or chondrogenic cells, are provided in an implant or scaffold for the regeneration of cartilaginous joint tissue. An effective cell dose may depend on the purity and the survival of the population. In some embodiments, an effective dose delivers a dose of cells of at least about 10², about 10³, about 10⁴, about 10⁵, about 10⁶, about 10⁷, about 10⁸, about 10⁹ or more cells, which chondrocytes or chondrogenic cells or chondrogenic progenitor cells may be present in the cell population at a concentration of about 1%, about 5%, about 10%, about 20%, about 30%, about 40%, about 50%, about 60%, about 70%, about 80%, about 90% or more.

The present application provides methods and compositions for the conversion of non-chondrogenic cells or non-chondrocyte into cells of the chondrogenic lineage or fate, such as chondrogenic cells, chondrogenic progenitor cells, or chondrocytes. The cells produced by the methods are useful in providing a source of fully differentiated and functional cells for research, transplantation, and the development of tissue engineering products for the treatment of human disease and traumatic injury repair.

### Tissue Engineering

Tissue engineering is the use of a combination of cells, engineering and materials methods, and suitable biochemical and physio-chemical factors to improve or replace biological functions. For example, cells may be implanted or seeded into an artificial structure capable of supporting three-dimensional tissue formation. These structures, referred to herein as a matrix or scaffold, allow cell attachment and migration, deliver and retain cells and biochemical factors, and enable diffusion of vital cell nutrients and expressed products. High porosity and adequate pore size are important to facilitate cell seeding and diffusion throughout the whole structure of both cells and nutrients. Biodegradability is often a factor since scaffolds may be absorbed by the surrounding tissues without the necessity of surgical removal. The rate at which degradation occurs has to coincide as much as possible with the rate of tissue formation, this means that while cells are fabricating their own natural matrix structure around themselves, the scaffold is able to provide structural integrity within the body and eventually it will break down leaving the neotissue, newly formed tissue which will take over the mechanical load. Injectability is also important for clinical uses.

Many different materials (natural and synthetic, biodegradable and permanent) have been investigated and can be used for tissue engineering matrices or scaffolds. Examples include Puramatrix, polylactic acid (PLA), polyglycolic acid (PGA) and polycaprolactone (PCL), and combinations thereof. Scaffolds may also be constructed from natural materials, e.g., proteins such as collagen, fibrin, etc; polysaccharidic materials, such aschitosan; alginate, glycosaminoglycans (GAGs) such as hyaluronic acid, etc. Functionalized groups of scaffolds may be useful in the delivery of small molecules (drugs) to specific tissues. Another form of scaffold under investigation is decellularised tissue extracts whereby the remaining cellular remnants/extracellular matrices act as the scaffold.

### Treatment Methods

The above-described cells, compositions, formulations, products, and methods can be used to treat various cartilage defects such as joint defects. Examples include (A) cartilage loss caused by various conditions (e.g., arthritis osteoarthritis, rheumatoid arthritis, childhood arthritis, fibromyalgia, gout, lupus, temporomandibular disorder, costochondritis, osteochondritis dissecans, chondrocalcinosis, post-traumatic osteoarthritis, and relapsing polychondritis), (B) large cartilage defects caused by various conditions, including cancer surgeries, congenital malformation (e.g., cleft palate, facial cleft. Treacher-Collins syndrome), trauma, and progressive deforming diseases, and (C) the stem cell-based therapy may be used to substitute any procedure involving cartilage graft, including those in plastic surgery such as cartilage graft rhinoplasty.

When used for tissue engineering or treatment methods, GATA3 proteins, activators, cells, regeneration products, and/or combinations of factors for lineage trans-differentiation can be provided for in vivo use in a solution, in which hydrating solutions, suspensions, or other fluids that contain the cells or factors that are capable of differentiating into cartilage.

Cartilage graft devices and compositions may be provided that are optimized in terms of one or more compositions, bioactivity, porosity, pore size, protein binding potential, degradability, or strength for use in both load bearing and non-load bearing cartilage grafting applications. Preferably, graft materials are formulated so that they promote one or more processes involved in cartilage or bone healing which can occur with the application of a single graft material; chondrogenesis, osteogenesis, osteoinduction, and osteoconduction. Chondrogenesis is the formation of new cartilaginous structures. Osteogenesis is the formation of new bone by the cells contained within the graft. Osteoinduction is a chemical process in which molecules contained within the graft (for example, the molecules secreted or released from the above-described cells, bone morphogenetic proteins, and TGF-β) convert the patient or other bone progenitor cells into cells that are capable of forming bone. Osteoconduction is a physical effect by which the matrix of the graft forms a scaffold on which bone forming cells in the recipient are able to form new bone.

The inclusion of GATA3, cells, and/or other factors described herein can be used to facilitate the replacement and filling of cartilage or bone material in and around pre-existing structures. In some embodiments, the cells produce chondrocytes first, followed by the deposition of extra cellular matrix and cartilage or bone formation. The grafts can provide an osteoconductive scaffold comprising calcium phosphate ceramics which provide a framework for the implanted progenitor cells and local skeletal stem cells or progenitors to differentiate into bone forming cells and deposit new bone. The use of calcium phosphate ceramics can provide for a slow degradation of the ceramic, which results in a local source of calcium and phosphate for bone formation. Therefore, new bone can be formed without calcium and phosphate loss from the host bone surrounding the defect site. Calcium phosphate ceramics are chemically compatible with that of the mineral component of bone tissues. Examples of such calcium phosphate ceramics include calcium phosphate compounds and salts, and combinations thereof.

In some embodiments, GATA3, cells, and/or other factors can be prepared as an injectable paste. A cellular suspension can be added to one or more cells to form an injectable hydrated paste. The paste can be injected into the implant site. In some embodiments, the paste can be prepared prior to implantation and/or store the paste in the syringe at sub-ambient temperatures until needed. In some embodiments, the application of the composite by injection can resemble a bone cement that can be used to join and hold bone fragments in place or to improve adhesion of, for example, a hip prosthesis, for replacement of damaged cartilage in joints, and the like. Implantation in a non-open surgical setting can also be performed.

In other embodiments, GATA3, cells, activators, and/or other factors can be prepared as formable putty. A cellular suspension can be added to one or more powdered minerals to form a putty-like hydrated graft composite. The hydrated graft putty can be prepared and molded to approximate any implant shape. The putty can then be pressed into place to fill a void in the cartilage, bone, tooth socket, or another site.

The methods described herein can be used for treating a cartilage or bone lesion, or injury, in a human or other animal subjects, comprising applying to the site a composition comprising GATA3, cells, activators, nucleic acids, vectors, and/or other factors described herein, which may be provided in combinations with cements, factors, gels, etc. As referred to herein such lesions include any condition involving cartilaginous and/or skeletal tissue that is inadequate for physiological or cosmetic purposes. Such defects include those that are congenital, the result of disease or trauma, and consequent to surgical or other medical procedures. Such defects include, for example, a cartilage or bone defect resulting from injury, defect brought about during the course of surgery, osteoarthritis, TMD, infection, malignancy, developmental malformation, and cartilage or bone breakages such as simple, compound, transverse, pathological, avulsion, greenstick and comminuted fractures. In some embodiments, a cartilage defect is a void in the cartilage that requires filling with a cartilage progenitor composition.

The cells described herein can also be genetically altered in order to enhance their ability to be involved in tissue regeneration or to deliver a therapeutic gene to a site of administration. To that end, a vector can be designed using the known encoding sequence for the desired gene, operatively linked to a promoter that is either pan-specific or specifically active in the differentiated cell type. Of particular interest are cells that are genetically altered to express a bone morphogenic protein, such as BMP-2 or BMP-4. See WO 99/39724. Production of these or other growth factors at the site of administration may enhance the beneficial effect of the administered cell, or increase the proliferation or activity of host cells neighboring the treatment site.

### Research Uses and Drug Screening

The cells described herein can also be used as a research or drug discovery tool, for example, to evaluate the phenotype of a genetic disease, e.g., to better understand the etiology of the disease, to identify target proteins for therapeutic treatment, to identify candidate agents with disease-modifying activity, e.g., to identify an agent that will be efficacious in treating the subject. For example, a candidate agent may be added to a cell culture comprising non-chondrocyte cells, chondrocytes, or chondrogenic cells with GATA3 or a GATA3 activator, and the effect of the candidate agent assessed by monitoring output parameters such as cell fate, lineage change, and survival, the ability to form cartilage, and the like, by methods described herein and in the art.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, an organic or inorganic molecule, nucleic acid, e.g., mRNA. DNA, etc., or a portion derived from such a cell component or combinations thereof. While most parameters will provide a quantitative readout, in some instances a semi-quantitative or qualitative result will be acceptable. Readouts may include a single determined value or may include the mean, median value or variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Examples of candidate agents include organic molecules comprising functional groups necessary for structural interactions, particularly hydrogen bonding, and typically include at least an amine, carbonyl, hydroxyl, or carboxyl group, frequently at least two of the functional chemical groups. The candidate agents can comprise cyclical carbon or heterocyclic structures and/or aromatic or polyaromatic structures substituted with one or more of the above functional groups. Candidate agents can be biomolecules, including peptides, polynucleotides, saccharides, fatty acids, steroids, purines, pyrimidines, derivatives, structural analogs, or combinations thereof. Further examples include pharmacologically active drugs, genetically active molecules, etc. Compounds of interest include chemotherapeutic agents, hormones or hormone antagonists, etc. Exemplary of the pharmaceutical agents suitable for this invention are those described in, “The Pharmacological Basis of Therapeutics,” Goodman and Gilman, McGraw-Hill, New York, N.Y., (1996). Ninth edition.

In an example, the cells and methods described herein are useful for screening candidate agents for activity in modulating cell conversion into cells of a chondrogenic lineage, e.g., chondrocytes or progenitor cells thereof. In screening assays for biologically active agents, the cells can be contacted with a candidate agent of interest in the presence of the cell reprogramming or differentiation system or an incomplete cell reprogramming or differentiation system, and the effect of the candidate agent is assessed by monitoring output parameters such as the level of expression of genes specific for the desired cell type, as is known in the art, or the ability of the cells that are induced to function like the desired cell type; etc. as is known in the art.

### Kits

The present disclosure provides a kit with packaging material and one or more components described therein. A kit typically includes a label or packaging insert including a description of the components or instructions for use in vitro, in vivo, or ex vivo, of the components therein. A kit can contain a collection of such components, e.g., the above-described polypeptide, activator, polynucleotide, nucleic acid, expression cassette, the expression vector (e.g., viral vector genome, expression vector, rAAV vector), cartilage regeneration product, scaffold, cell, composition and formulation optionally a second active agent such as a compound, therapeutic agent, drug or composition.

A kit refers to a physical structure that contains one or more components of the kit. Packaging material can maintain the components in a sterile manner and can be made of material commonly used for such purposes (e.g., paper, glass, plastic, foil, ampules, vials, tubes, etc).

A label or insert can include identifying information of one or more components therein, dose amounts, and clinical pharmacology of the active ingredients(s) including mechanism of action, pharmacokinetics, and pharmacodynamics. A label or insert can include information identifying manufacture, lot numbers, manufacture location and date, and expiration dates. A label or insert can include information on a disease (e.g., a joint disorder) for which a kit component may be used. A label or insert can include instructions for a clinician or subject for using one or more of the kit components in a method, use or treatment protocol, or therapeutic regimen. Instructions can include dosage amounts, frequency of duration, and instructions for practicing any of the methods, uses, treatment protocols, or prophylactic or therapeutic regimens described herein.

A label or insert can include information on potential adverse side effects, complications, or reactions, such as a warning to a subject or clinician regarding situations where it would not be appropriate to use a particular composition.

### Definitions

A nucleic acid or polynucleotide refers to a DNA molecule (e.g., but not limited to, a cDNA or genomic DNA), an RNA molecule (e.g., but not limited to, an mRNA), or a DNA or RNA analog. A DNA or RNA analog can be synthesized from nucleotide analogs. The nucleic acid molecule can be single-stranded or double-stranded. An “isolated nucleic acid” is a nucleic acid the structure of which is not identical to that of any naturally occurring nucleic acid or to that of any fragment of a naturally occurring genomic nucleic acid. The term, therefore, covers, for example, (a) a DNA that has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein.

As mentioned above, a nucleic acid sequence can be a DNA or RNA. The terms “RNA,” “RNA molecule,” and “ribonucleic acid molecule” are used interchangeably herein, and refer to a polymer of ribonucleotides. The term “DNA”. “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA also can be chemically synthesized. DNA and RNA can be single-stranded (i.e., ssRNA and ssDNA, respectively) or multi-stranded (e.g., double-stranded, i.e., dsRNA and dsDNA, respectively).

The terms “polypeptide” or “protein” are used interchangeably herein to refer to a polymer of amino acid residues and their derivatives. The terms also apply to amino acid polymers in which one or more amino acid residues are an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally occurring and non-recombinant cell; or it is produced by a genetically engineered or recombinant cell, and comprise molecules having the amino acid sequence of the native protein, or molecules having deletions from, additions to, and/or substitutions of one or more amino acids of the native sequence.

The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains.

The term “isolated polypeptide” refers to a polypeptide that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic, or research use.

As used herein, the term “homologous,” or “homology,” refers to two or more reference entities (e.g., a nucleic acid or polypeptide sequence) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid, or fragment thereof. “substantial homology” or “substantial similarity,” means that when optimally aligned with appropriate insertions or deletions with another polypeptide, nucleic acid (or its complementary strand) or fragment thereof, there is sequence identity in at least about 70% to 99% of the sequence. The extent of homology (identity) between two sequences can be ascertained using a computer program or mathematical algorithm known in the art. Such algorithms that calculate percent sequence homology (or identity) generally account for sequence gaps and mismatches over the comparison region or area.

As used herein, the term “overexpressing.” “overexpress,” “overexpressed.” or “overexpression.” when referring to the production of a nucleic acid or a protein in a host cell means that the nucleic acid or protein is produced in greater amounts than it is produced in its naturally occurring environment. It is intended that the term encompasses overexpression of endogenous, as well as exogenous or heterologous nucleic acids and proteins. As such, the terms and the like are intended to encompass increasing the expression of a nucleic acid or a protein in a cell to a level greater than that the cell naturally contains. In certain embodiments, the expression level or amount of the nucleic acid or protein in a cell is increased by at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level or amount that the cell naturally contains.

As used herein, the term “administering” refers to the delivery of compositions of the present disclosure by any suitable route. Cells can be administered in a number of ways including, but not limited to, parenteral (such a term referring to intravenous and intra-arterial as well as other appropriate parenteral routes), intrathecal, intraventricular, intraparenchymal, intracisternal, intracranial, intrastriatal, intranigral, intranasal, intraperitoneal, intramuscular, subcutaneous, intradermal, transdermal, or transmucosal administration, among others which term allows cells to migrate to the ultimate target site where needed. Multiple units of cells can be administered simultaneously or consecutively (e.g., over the course of several minutes, hours, or days) to a patient.

The terms “grafting” and “transplanting” and “graft” and “transplantation” are used to describe the process by which cells are delivered to the site where the cells are intended to exhibit a favorable effect, such as repairing damage to a patient's bone or cartilage. Cells can also be delivered in a remote area of the body by any mode of administration as described above, relying on cellular migration to the appropriate area to effect transplantation.

The term “therapeutic composition” or “pharmaceutical composition” refers to the combination of an active agent with a carrier, inert or active, making the composition especially suitable for diagnostic or therapeutic use in vivo or ex vivo.

As used herein, “therapeutic cells” refers to a cell population that ameliorates a condition, disease, and/or injury in a patient. Therapeutic cells may be autologous (i.e., derived from the patient), allogeneic (i.e., derived from an individual of the same species that is different from the patient), or xenogeneic (i.e., derived from a different species than the patient). Therapeutic cells may be homogenous (i.e., consisting of a single cell type) or heterogeneous (i.e., consisting of multiple cell types). The term “therapeutic cell” includes both therapeutically active cells as well as progenitor cells capable of differentiating into a therapeutically active cell.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for the delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

As used herein, the terms “subject” and “subjects” may refer to any vertebrate, including, but not limited to, a mammal (e.g., cow, pig, camel, llama, horse, goat, rabbit, sheep, hamster, guinea pig, cat, dog, rat, and mouse, a non-human primate (for example, a monkey, such as a cynomolgus monkey, chimpanzee, etc.) and a human). The term “subject” includes human and non-human animals. The preferred subject for treatment is a human. As used herein, the terms “subject” and “patient” are used interchangeably irrespective of whether the subject has or is currently undergoing any form of treatment. In one embodiment, the subject is a human. In another embodiment, the subject is an experimental, non-human animal or animal suitable as a disease model.

The term “patient” is used herein to describe an animal, preferably a human, to whom treatment, including prophylactic treatment, with the cells according to the present invention, is provided. The term “donor” is used to describe an individual (animal, including a human) who or which donates cells or tissue for use in a patient.

The term “primary culture” denotes a mixed cell population of cells from an organ or tissue within an organ. The word “primary” takes its usual meaning in the art of tissue culture.

A “tissue” refers to a group or layer of similarly specialized cells which together perform certain special functions.

The terms “treatment”, “treating”. “treat” and the like are used herein to generally refer to obtaining a desired pharmacologic and/or physiologic effect. The effect may be prophylactic in terms of completely or partially preventing a disease or symptom thereof and/or maybe therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. The terms “prevent.” “preventing,” “prevention,” “prophylactic treatment” and the like refer to reducing the probability of developing a disorder or condition in a subject, who does not have, but is at risk of or susceptible to developing a disorder or condition. “Ameliorating” generally refers to the reduction in the number or severity of signs or symptoms of a disease or disorder.

A “prophylactic treatment” includes a treatment administered to a subject who does not display signs or symptoms of a condition such that treatment is administered for the purpose of diminishing, preventing, or decreasing the risk of developing the condition. A “therapeutic treatment” includes a treatment administered to a subject who displays symptoms or signs of a condition and is administered to the subject for the purpose of reducing the severity or progression of the condition. A therapeutic treatment can also partially or completely resolve the condition.

An “effective amount” generally means an amount that provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and the amount of time since the injury occurred or the disease began. One skilled in the art will be able to determine the effective amount for a given subject based on these considerations which are routine in the art. As used herein, “effective dose” means the same as “effective amount.”

As used herein, the term “stem cells” refers to cells with the ability to both replace themselves and to differentiate into more specialized cells. Their self-renewal capacity generally endures for the lifespan of the organism. A pluripotent stem cell can give rise to all the various cell types of the body. A multipotent stem cell can give rise to a limited subset of cell types. For example, a hematopoietic stem cell can give rise to the various types of cells found in blood, but not to other types of cells. Multipotent stem cells can also be referred to as somatic stem cells, tissue stem cells, lineage-specific stem cells, and adult stem cells. The non-stem cell progeny of multipotent stem cells are progenitor cells (also referred to as restricted-progenitor cells). Progenitor cells give rise to fully differentiated cells, but a more restricted set of cell types than stem cells. Progenitor cells also have comparatively limited self-renewal capacity; as they divide and differentiate they are eventually exhausted and replaced by new progenitor cells derived from their upstream multipotent stem cell.

The term “skeletal stem cell” refers to a multipotent and self-renewing cell capable of generating bone marrow stromal cells, skeletal cells, and chondrogenic cells. By self-renewing, it is meant that when they undergo mitosis, they produce at least one daughter cell which is a skeletal stem cell. By multipotent it is meant that it is capable of giving rise to progenitor cells (skeletal progenitors) that give rise to all cell types of the skeletal system. They are not pluripotent, that is, they are not capable of giving rise to cells of other organs in vivo.

Skeletal stem cells can be reprogrammed from non-skeletal cells, including without limitation mesenchymal stem cells, and adipose tissue containing such cells. Reprogrammed cells may be referred to as induced skeletal stem cells, or iSSC. “iSSC” arise from a non-skeletal cell by experimental manipulation. Induced skeletal cells have characteristics of functional SSCs derived from nature, that is, they can give rise to the same lineages.

Suture stem cells (SuSCs) refer to a population of skeletal stem cells from the suture mesenchyme that exhibit long-term self-renewal, clonal expansion, and multipotency. These SuSCs reside in the suture midline and serve as the skeletal stem cell population responsible for calvarial development, homeostasis, injury repair, and regeneration Suture stem cells are the stem cell population that is naturally programmed to form intramembranous bones during craniofacial skeletogenesis.

Chondrocytes (cartilage cells) refer to cells that are capable of expressing characteristic biochemical markers of chondrocytes, including but not limited to collagen type II, chondroitin sulfate, keratin sulfate, and characteristic morphologic markers of a chondrocyte, including but not limited to the rounded morphology observed in culture, and able to secrete collagen type II, including but not limited to the generation of tissue or matrices with properties of cartilage in vitro.

As used herein, the phrase “maintaining stem cells” refers not just to culturing the stem cells in a manner preserving their viability, but also to retaining their functionality as stem cells, that is, to being self-renewing and capable of giving rise to the full range of progenitor lineages appropriate to the particular type of stem cell (these two functions together “regenerative activity”). One way of demonstrating that stem cells have been successfully maintained is through an engraftment experiment in which all the appropriate cell types (bearing a genetic marker distinguishing them from the host) are observed to arise from the graft and remain present over an extended period, for example, 4 months.

As used herein, the phrase “expanding stem cells” refers not just to maintaining the stem cells but to culturing the stem cells in a manner that the number of stem cells in the culture increases. One way of demonstrating that stem cells have been successfully expanded is an engraftment experiment comparing the percentage of donor-derived cells obtained from transplants of cultured and freshly isolated stem cells. The comparison is based on transplanting the same number of freshly isolated stem cells as were originally placed in culture. An increased percentage of donor-derived cells in the recipients of the cultured stem cells as compared to in the recipients of the freshly-isolated stem cells is consistent with the successful expansion of the stem cells in culture.

A “marker” or “biomarker” is a molecule useful as an indicator of a biologic state in a subject. The marker or biomarkers disclosed herein can be polypeptides that exhibit a change in expression or state, which can be correlated with the development, differentiation, or fate of a cell. In addition, the markers disclosed herein are inclusive of messenger RNAs (mRNAs) encoding the marker polypeptides, as the measurement of a change in the expression of an mRNA can be correlated with changes in the expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

In the context of skeletal stem cells, a “marker” or “biomarker” means that, in cultures or tissues comprising cells that have been programmed to become skeletal stem cells, the markers are mRNAs or proteins used to identify and isolate the stem cell population. It will be understood by those of skill in the art that the stated expression levels reflect detectable amounts of the marker protein or mRNA on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still, be characterized as “positive.”

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

A “non-naturally encoded amino acid” refers to an amino acid that is not one of the common amino acids or pyrolysine, pyrroline-carboxy-lysine, or selenocysteine. Other to that may be used synonymously with the term “non-naturally encoded amino acid” are “non-natural amino acid.” and “unnatural amino acid.” “non-naturally-occurring amino acid,” and variously hyphenated and non-hyphenated versions thereof. The term “non-naturally encoded amino acid” also includes, but is not limited to, amino acids that occur by modification (e.g., post-translational modifications) of a naturally encoded amino acid (including but not limited to, the 20 common amino acids or pyrrolysine, pyrroline-carboxy-lysine, and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Unless otherwise stated, the term “about” means within an acceptable error range for the particular value.

The term “differentiated cell” encompasses any somatic cell that is not, in its native form, pluripotent, as that term is defined herein. Thus, the term a “differentiated cell” also encompasses cells that are partially differentiated, such as multipotent cells, or cells that are stable, non-pluripotent partially reprogrammed, or partially differentiated cells, generated using any of the compositions and methods described herein. In some embodiments, a differentiated cell is a cell that is a stable intermediate cell, such as a non-pluripotent, partially reprogrammed cell. It should be noted that placing many primary cells in culture can lead to some loss of fully differentiated characteristics. Thus, simply culturing such differentiated or somatic cells does not render these cells non-differentiated cells (e.g., undifferentiated cells) or pluripotent cells. The transition of a differentiated cell (including stable, non-pluripotent partially reprogrammed cell intermediates) to pluripotency requires a reprogramming stimulus beyond the stimuli that lead to a partial loss of differentiated character upon placement in culture. Reprogrammed and, in some embodiments, partially reprogrammed cells, also have the characteristic of having the capacity to undergo extended passaging without loss of growth potential, relative to parental cells having lower developmental potential, which generally have the capacity for only a limited number of divisions in culture. In some embodiments, the term “differentiated cell” also refers to a cell of a more specialized cell type (i.e., decreased developmental potential) derived from a cell of a less specialized cell type (i.e., increased developmental potential) (e.g., from an undifferentiated cell or a reprogrammed cell) where the cell has undergone a cellular differentiation process.

The terms “proliferation” and “expansion” as used interchangeably herein refer to an increase in the number of cells of the same type by division. The term “differentiation” refers to a developmental process whereby cells become specialized for a particular function, for example, where cells acquire one or more morphological characteristics and/or functions differently from that of the initial cell type. The term includes both lineage commitment and terminal differentiation processes. Differentiation may be assessed, for example, by monitoring the presence or absence of lineage markers, using immuno-histochemistry or other procedures known to a skilled in the art. Differentiated progeny cells derived from progenitor cells may be, but are not necessarily, related to the same germ layer or tissue as the source tissue of the stem cells. For example, neural progenitor cells and muscle progenitor cells can not differentiate into hematopoietic cell lineages.

### EXAMPLES

### Example 1 Materials and Methods

All experiments were performed according to the guidelines, and Intramural Animal Use and Care Committees of Forsyth Institute and the University of Rochester. This study is compliant with the ARRIVE guidelines (Animal Research Reporting of In vivo Experiments). Materials are freely distributed upon request to the qualified academic investigator for non-commercial research and mouse strains are available according to the NIH Grant Policy on Sharing of Model Organisms for Biomedical Research.

### Study Design

This study was designed to elucidate the mechanism underlying skeletal cell fate determination. Using several mouse models with cell type-specific disruption of Gpr177 and β-catenin and activation of Wnt5a, it was found that Wnt signaling mediated by β-catenin-dependent transcription is not associated with skeletal lineage specification. The ectopic chondrogenesis caused by β-catenin deficiency is independent of its transcriptional output. Transplantation further revealed critical alteration in stem cell characteristics associated with the generation of cartilage and bone, suggesting nonclassical β-catenin signaling is essential for skeletal cell fate determination.

Using an unbiased genomic approach, inventors characterized gene expression profiling linked to the nonclassical effects of β-catenin and identified GATA3 associated with skeletal cell fate switching. A mouse model was generated to demonstrate that GATA3 is sufficient to alter skeletal lineage commitment. Micro mass culture further indicated the dependence of GATA3 in promoting chondrogenesis caused by the loss of β-catenin. For scientific reproducibility, all studies were performed and repeated with proper controls, including mouse embryos carrying appropriate transgene(s). At least three independent experiments were performed for each study. No randomization, statistical method to predetermine the sample size, and inclusion/exclusion criteria defining criteria for samples were used.

### Animals and Models

The Twist2tm.1(cre)Dor (Dermol-Cre), Tg(KRTJ4-cre)1Amc (K14-Cre), Gpr177Fx, R26StopWnt5a, R26StopGATA3, Cinnb1tm2Kem (β-cateninFx), β-catenin^{dm}, and Prkdcscid (SCID) mouse strains, the generation of Gpr177^{Dermol} and Gpr177^{K14} models, and genotyping methods were reported previously (25, 26, 29, 47-54). The Dermol-Cre mice were crossed with β-cateninFx and β-catenin^{dm} mice to create β-cat^{Dermol} and β-cat^{Dermol}ΔTF models, respectively. To generate the Gpr177^{Dermol/K14} model, K14-Cre and Dermol-Cre transgenes were bred into the Gpr177Fx/Fx background. The Wnt5a^{Dermol-OE} and GATA3^{Dermol-OE} models were generated by crossing mice heterozygous for the Dermol-Cre allele with mice homozygous for the R26StopWnt5a or R26StopGATA3 allele. Both male and female mice were used in this study. Care and use of experimental animals described in this work comply with the guidelines and policies of IACUC at the Forsyth Institute and the University Committee on Animal Resources at the University of Rochester

### Cell Isolation and Transplantation

Primary suture mesenchymal cells containing SuSCs were isolated from mouse calvaria as described (12, 15, 55). Briefly, an approximately 1.5 mm width tissue containing sagittal suture at postnatal day 5 (P5) and its adjacent parietal bones were dissected, followed by separation of the parietal bone parts. Next, the suture parts were incubated with 0.2% collagenase in PBS at 37° C., for 1.5 hours. The dissociated cells were filtered, and then re-suspended in DMEM media for transplantation analysis. The transplantation of freshly isolated cells of control. β-cat^{Dermol}, and β-cat^{Dermol}ΔTF into the kidney capsule was performed as described (12, 15). The isolation of primary cells from the calvarial bone was performed as described (56). The isolated calvarial cells were cultured in the minimum essential αMEM medium containing 10% fetal bovine serum. The addition of ascorbic acid (50 μg/ml) and 4 mM β-glycerophosphate promotes the differentiation of 2.5-10⁵ calvarial cells seeded in 24-well plates with infection of control or Gata3-expressing lentiviruses at MOI=1, followed by alcian blue staining in 3 weeks (21, 57). For alcian blue staining, cells were fixed in a solution containing 30% ethanol, 0.4% paraformaldehyde, and 4% acetic acid for 15 min at room temperature, followed by incubation with 0.05% alcian blue staining solution in 75% ethanol:0.1M hydrochloride (4:1) overnight at 37° C.

### Histology and Staining

Sample preparation, fixation, and embedding for paraffin sections and histological analysis were performed as described (58, 59). Samples were subject to hematoxylin and eosin staining for histology, alcian blue staining, von Kossa staining, or immunological staining with avidin: biotinylated enzyme complex (15, 60). The in situ hybridization analyses were performed as described (60). In brief. DNA plasmids, containing Col2a1, Col10a1, Col1a1, and Osteocalcin (OC) cDNAs, were linearized for in vitro transcription using T3 or T7 RNA polymerase (PROMEGA, Wisconsin, WI, USA) to generate digoxigenin-labeled RNA probes for in situ hybridization. Sections were then incubated with the RNA probes, followed by recognition with an alkaline phosphatase-conjugated anti-digoxigenin antibody (ROCHE, Indianapolis, IN, USA). To visualize the bound signals, samples were incubated with BM-purple (ROCHE) for 4 to 5 hours. The immunological staining was visualized by enzymatic color reaction or fluorescence according to the manufacturer's specification (VECTOR LABORATORIES, Burlingame, CA). The sections underwent an antigen retrieval process by incubating with antigen unmasking solution (H3300, VECTOR LABORATORIES) in pressured cooking for 10 min. Mouse monoclonal antibodies α-β-catenin N (ALX-804-060, ENZO LIFE SCIENCES, Exeter, UK; 1:200), α-β-catenin C (610153, BD TRANSDUCTION LABORATORIES, Franklin Lakes, NJ, USA; 1:200), Aggrecan (MABT84, MERCK MILLIPORE, Burlington, MA, 1:100). Col2a1 (MA5-13026, INVITROGEN, Waltham, MA; 1:100), and OB-cadherin (32-1700, INVITROGEN; 1:200), rabbit polyclonal antibodies Osterix (ab22552, Abcam, Cambridge, MA, USA; 1:500) and LEF1 (bs-1843R, Bioss, Beijing, China; 1:200), and rabbit monoclonal antibody Gata3 (ab199428, ABCAM; 1:250) were used for immunological staining. Images were taken using a ZEISS AXIO OBSERVER microscope (CARL ZEISS, Thornwood, NY), LEICA DM2500 microscope with a DFC7000T digital imaging system (LEICA BIOSYSTEMS Inc., Buffalo Grove, IL), or NIKON ECLIPSE Ts2 microscope with Insight CMOS digital camera (NIKON, Melville, NY).

### RNA-Seq and Statistics

The calvarial mesenchymes isolated from E14.5 control, β-cat^{Dermol}, and β-cat^{Dermol}ΔTF mice were subject to RNA-seq analysis for the comparison of gene expression profiles using the ION TORRENT PGM sequencer. The differentially expressed genes (DEGs) in the β-cat^{Dermol} and β-cat^{Dermol}ΔTF mutant mesenchymes were identified by the use of the R package edgeR. The criteria to select DEGs were >2-fold changes and average gene counts >10. The Interactome/Upstream Regulator analysis assay was performed using METACORE software (34). R software version 3.2.1 or MICROSOFT EXCEL 2010 was used for statistical analysis. The significance was determined by two-sided Student t-tests. A p-value of less than 0.05 was considered statistically significant. Before performing the t-tests, the normality of the data distribution was first validated by the Shapiro-Wilk normality test. To quantify the AB+ area, the RGB image was split into single channels. The resulting grayscale image with the red channel was used for quantification. The positive area is selected by threshold 0-62.

### Example 2 Wnt Signaling Mediated by β-Catenin-Dependent Transcription is not Associated with Skeletal Lineage Specification

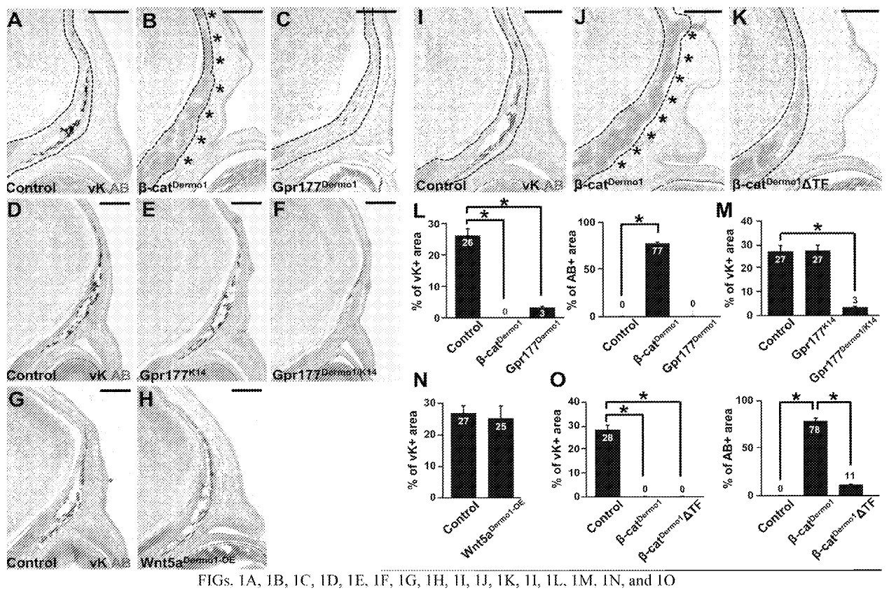

Ectopic chondrogenesis caused by the loss of β-catenin (22, 23) prompted inventors to elucidate the mechanism underlying the skeletal cell fate switching. In the Gpr177 (mouse ortholog of Drosophila Wntless) deficient model, inventors found impaired Wnt secretion in the signal-producing mesenchymal cells causing defects in calvarial and skeletal bone formation, similar to the loss of β-catenin (22, 23, 25). In the skeletogenic mesenchyme, the loss of Gpr177-mediated Wnt secretion disrupted bone ossification but did not cause ectopic chondrogenesis which is evident in the β-catenin deficient calvaria (FIG. 1A-C, L, *; p<0.001, n≥3, mean±SD; two-sided student t-test). Although osteogenesis was similarly defective in both mutants, ectopic chondrogenesis only occurred in the β-cat^{Dermol} but not Gpr177^{Dermol} mice when gene deletion is activated by Dermol-Cre (aka Twist2-Cre) in the mesenchyme (FIG. 1A-C, L. *; p<0.001, n≥3, mean±SD; two-sided student t-test). It was hypothesized that this discrepancy may be attributed to 1) the presence of Wnt secreted by nearby epidermal tissue, 2) the effect of noncanonical Wnt or 3) the alternative function, e.g., cell adhesion or LEF/TCF independent transcription of β-catenin.

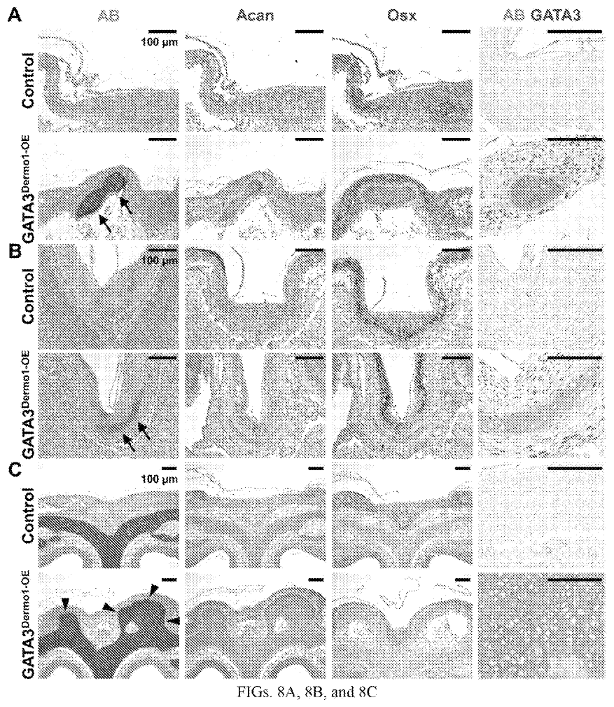

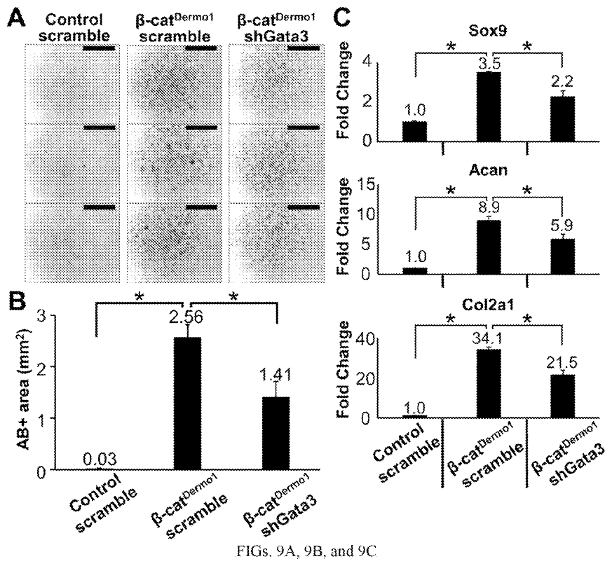

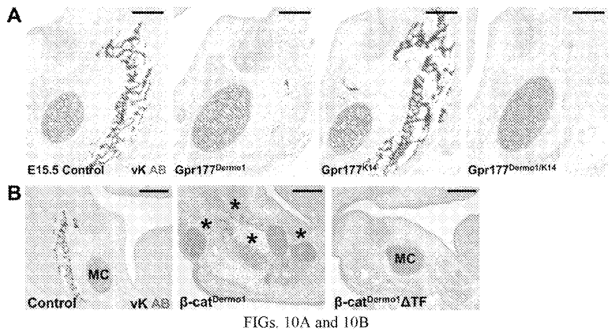

First, epidermal secretion of Wnt may be able to maintain skeletal cell fate via paracrine signaling effects upon removal of mesenchymal Wnt in the Gpr177^{Dermol} mutant. To test this possibility, inventors examined if epidermal Wnt contributes to the maintenance of skeletal cell fate by creating mice with epidermal loss of Gpr177 (Gpr177^{K14}; n=6). At E15.5, the epidermal deletion of Gpr177 to eliminate its supply of Wnt did not induce ectopic chondrogenesis (FIG. 1D. E). Next, mice with the loss of Gpr177 in both skeletogenic mesenchymal cells and epidermal cells (Gpr177^{Dermol/K14}; n=8) were generated. These double mutants still did not exhibit ectopic chondrogenesis (FIG. 1F). Although bone ossification was disrupted, no ectopic chondrogenesis was detected in the Gpr177^{Dermol/K14} mutant calvaria (FIG. 1M, *; p<0001, n≥3, mean±SD; two-sided student t-test and FIG. 10A). Immunostaining analysis supported the efficiency of Cre-mediated disruption of Gpr177 in these models (FIG. 11A). Ectopic chondrogenesis were not detected in the limb although a clear delay in endochondral ossification was associated with mesenchymal deletion of Gpr177 (FIG. 12A, B). The results suggested the cell fate switching was not caused by the loss of mesenchymal and epidermal Wnts.

Second, β-catenin deficiency affects canonical Wnt signaling while the secretion of all Wnt requires Gpr177 whose disruption impairs canonical and noncanonical Wnts (26). The balance of these two pathways may be critical for skeletal lineage commitments. Noncanonical signaling is known to counterbalance the canonical signaling of Wnt (27). Therefore, another possibility is that the loss of β-catenin/canonical Wnt signaling led to an elevation of noncanonical Wnt signaling, responsible for the alteration of skeletal cell fate. Therefore, a mouse model with transgenic expression of Wnt5a in the skeletogenic mesenchyme (FIG. 11B; n=5) was generated. However, overexpression of Wnt5a failed to detect any ectopic chondrogenesis in the calvaria and limbs (FIG. 1G-H, N, and FIG. 12C). These results suggested that the balance of canonical and noncanonical Wnt signaling may not be associated with skeletal cell fate switching.

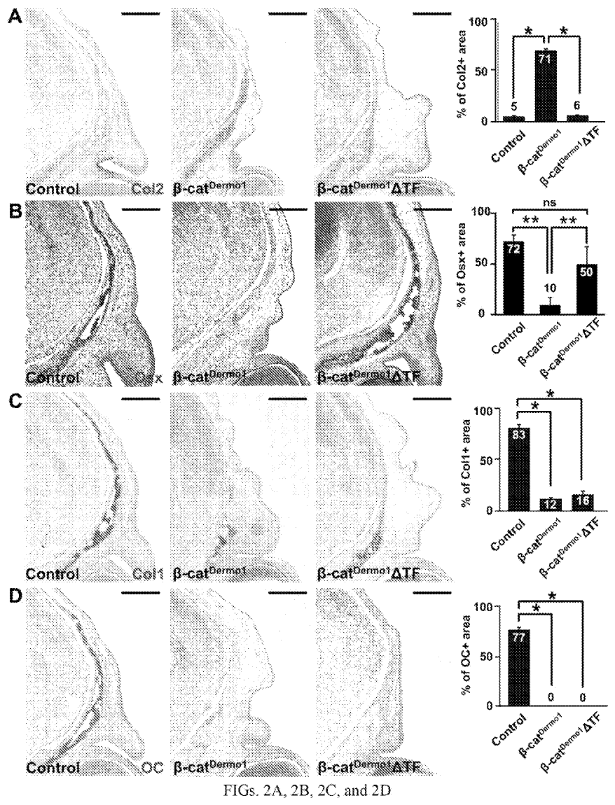

Third, as β-catenin possesses additional functions, e.g., cell adhesion, skeletal lineage specification may be independent of canonical Wnt signaling. To rigorously examine the requirement of canonical Wnt signaling for skeletal fate determination, mice deficient for the transcriptional output of β-catenin in the endogenous locus were created. This mutant, containing one amino acid substitution in the first armadillo repeat (D164A) and deletion of the C-terminus (AC), affected the transcription function but not the cell adhesion function of β-catenin (28). Cell-cell interaction mediated by β-catenin remains intact in the β-catenin^{dm} allele (29). Using this allele. β-cat^{Dermol}ΔTF mutants in which only β-catenin-dependent transcription is deficient were created. Both β-cat^{Dermol}ΔTF and β-cat^{Dermol} mutants exhibited bone ossification defects (FIG. 1I-K; FIG. 1O, *; p<0.001, n≥3, mean±SD; two-sided student t-test). However, ectopic chondrogenesis was not evident in the calvarial and mandible regions of 3-cat^{Dermol}ΔTF, highly reminiscent of the Gpr177 deficient mutant (FIG. 1I-K, FIG. 10B; FIG. 1O, *; p<0.001, n≥3, mean±SD; two-sided student t-test). Molecular characterizations with various markers were carried out to examine the cell types affected by the mutations. The ectopic chondrocytes expressing type 2 collagen (Col2) were present in the β-cat^{Dermol} but not in control and β-cat^{Dermol}ΔTF mice (FIG. 2A). This skeletogenic region normally formed calvarial bones via intramembranous ossification with the presence of osteoblast cells positive for osterix (Osx), type 1 collagen (Col1), and osteocalcin (OC) in the E15.5 control (FIG. 2B-D). The disruption of β-catenin-dependent transcription had no apparent effects on the Osx+ progenitor but significantly impaired the differentiation of Col1+ and OC+ osteoblast cells similar to the β-cat^{Dermol} mutant (FIG. 2C, D). These findings implied that the skeletal lineage commitment is orchestrated by β-catenin but independent of transcriptional activation.

### Example 3 Nonclassical β-Catenin Signaling in Skeletal Cell Fate Determination

To determine the nature of mutation affecting the function in the β-cat^{Dermol} no and β-cat^{Dermol}ΔTF mice, immunostaining analyses were performed. Using antibodies recognizing different domains of β-catenin, inventors showed the N-terminal region remains intact in the skeletogenic mesenchyme of β-cat^{Dermol}ΔTF (FIG. 3A). However, the C-terminal region is deleted in both mutants (FIG. 3B). The results demonstrated that the C-terminal transcriptional activation domain is abolished while other regions critical for cell adhesion are not affected in the β-catenin^{dm} allele. The mutation also impaired the activation of canonical Wnt targets whose expression is dependent on β-catenin-mediated transcription. Immunostaining of LEF1, TCF7, and DKK1 was reduced in both β-cat^{Dermol} and β-cat^{Dermol}ΔTF skeletogenic mesenchyme (FIG. 3D-F). The skeletogenic mesenchyme expresses OB-cadherin/cadherin-11 known to interact with β-catenin upon epithelial-mesenchymal transition to mediate cell adhesion and migration during bone metastasis (30, 31). The staining of OB-cadherin suggested that cell-cell interaction is disrupted in the β-cat^{Dermol} but unaffected in the β-cat^{Dermol}ΔTF mutants (FIG. 3C).

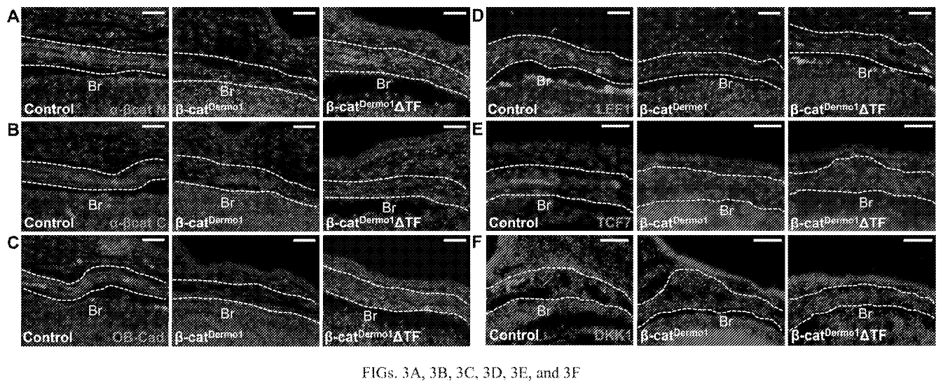

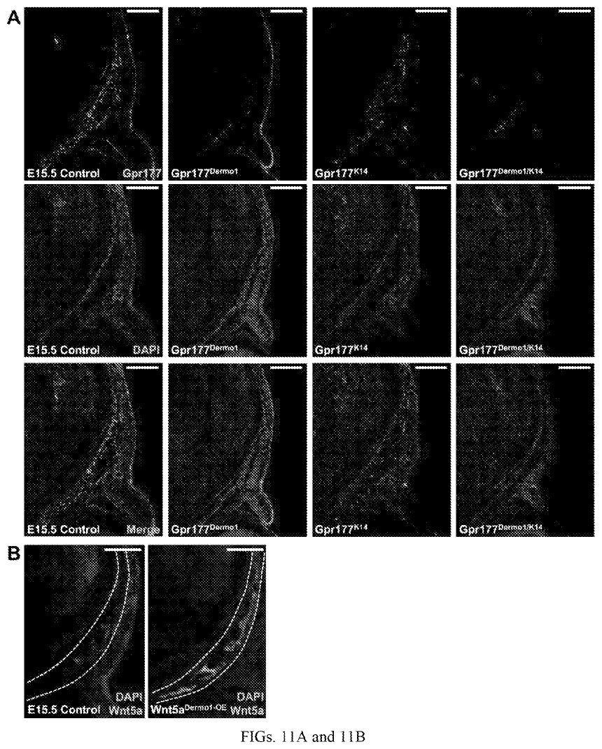

To test the alteration of skeletal cell fate, inventors examined their regenerative characteristics using transplantation assays. Inventors previously showed the ability of a single SuSC (sture stem cells—skeletal stem cells within calvarial suture mesenchyme) to generate bone thereby examining their stemness using in vivo clonal expansion analysis in the kidney capsule (12, 15). Therefore, kidney capsule transplantation was performed to examine the stem cell characteristics affected by the β-catenin mutations. Cells were isolated from the P5 β-cateninFx/Fx and β-catenin^{dm}/Fx calvarial sutures. The β-cateninFx/Fx, and β-catenin^{dm}/Fx suture cells were infected by lentivirus-Cre to generate β-cat-null and β-catΔTF cells, followed by immediately implanted into the kidney capsule (FIG. 4; n=3, 100% transplantation success rate). At post-transplantation of two weeks, the implanted site was evaluated by histological and immunological staining to examine the stem cell-generated tissue structure and cell types. First, the efficacy of Cre-mediated deletion by lentiviral infection was analyzed by fluorescent images of the transplanted kidney in the whole mount and section (FIG. 13). As expected, the expression of the RFP reporter associated with lentivirus-Cre was detected in the β-cat-null and β-catΔTF but not in control transplants (FIG. 13C). The β-catenin staining was positive in the control but lost in the β-cat-null and β-catΔTF (FIG. 13D). Next, consistent with previous findings (12, 15), transplantation of 5×10⁴ control cells generated tissues resembling the calvarial bone (FIG. 4A) containing cells positive for Osx (FIG. 4B) but negative for alcian blue, and chondrocyte markers, Acan and Col2 (FIG. 4C-E). The implanted β-cat-null cells generated cartilages containing cells positive for alcian blue, Acan, and Col2, but not Osx (FIG. 4A-E). However, the tissue generated by β-catΔTF cells was negative for Osx, Acan, Col2, or alcian blue (FIG. 4A-E). The results from the transplantation assays supported mouse genetic studies in the calvaria, suggesting that β-catenin-dependent transcription is essential for osteogenesis. However, skeletal cell fate determination is independent of the transcriptional output of β-catenin.

### Example 4 β-Catenin-Dependent Transcription in Endochondral Ossification and Palatogenesis

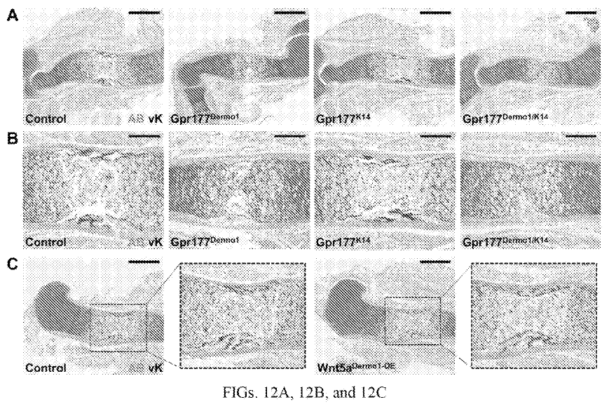

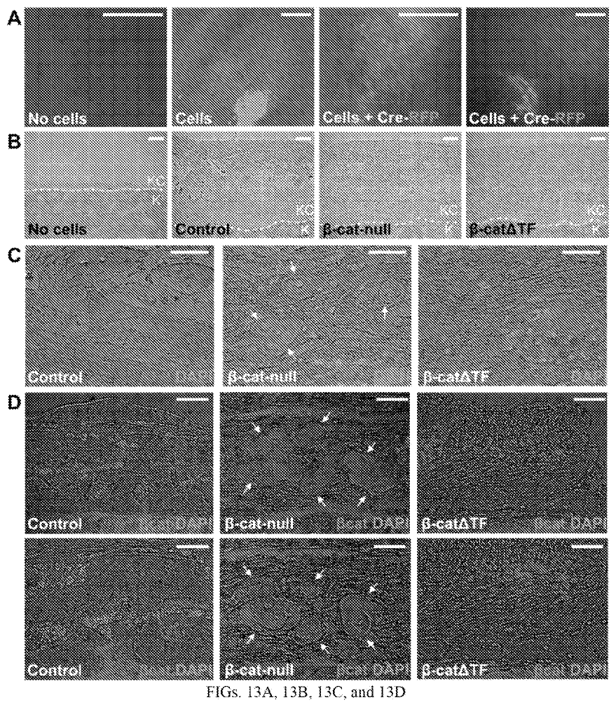

Canonical Wnt signaling mediated by β-catenin-dependent transcription is known to regulate the development of craniofacial as well as body skeletons. To further dissect the requirement of β-catenin for endochondral ossification, limb development at an early embryonic stage was examined. In the humeri, alcian blue staining, and expression of Col2 and Col10 indicated that chondrogenesis is severely delayed in both β-cat^{Dermol} and β-cat^{Dermol}ΔTF mutants (FIG. 14A-C). The delay caused a cascade effect on subsequent osteoblast differentiation as evidenced by the staining of Osx and Col1 (FIG. 14D, E). No ectopic chondrogenesis was detectable in the β-catenin mutants. The results demonstrated that β-catenin-mediated transcription is essential for chondrocyte maturation and subsequent ossification. The highly similar defects exhibited in these two mutants also suggested a complete disruption of canonical Wnt signaling in the β-cat^{Dermol}ΔTF limb. Similar conclusions are supported by the development of the cleft palate in both mutants (FIG. 15). Consistent with previous reports (22, 23), the findings indicated canonical Wnt/β-catenin signaling essential for endochondral ossification and palatogenesis. Therefore, no cell fate switching detected in the calvaria of β-cat^{Dermol}ΔTF was not attributed to incomplete abrogation of Wnt/β-catenin signaling.

### Example 5 Gene Expression Profiling of β-Cat^{Dermol} and β-Cat^{Dermol}ΔTF

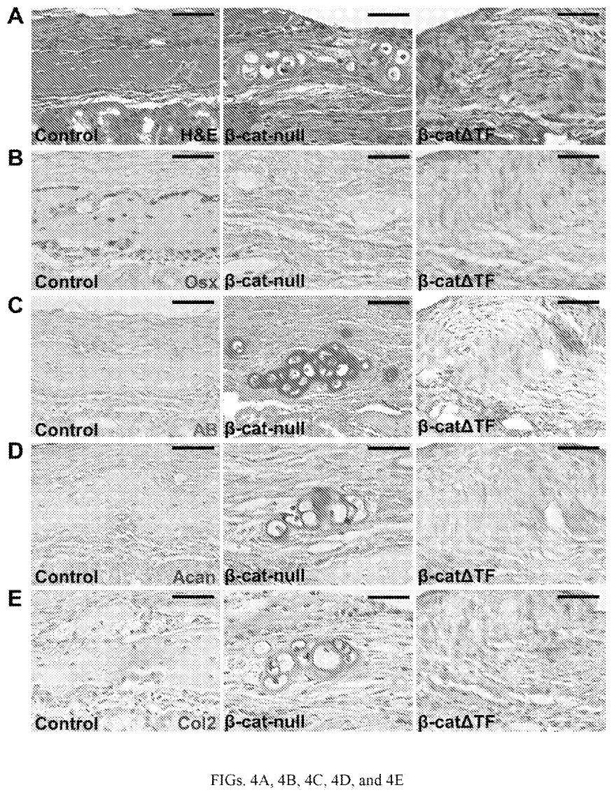

To further delineate the nonclassical signaling mechanism underlying skeletal lineage specification orchestrated by β-catenin-dependent but transcription-independent function, inventors performed RNA-seq analysis comparing gene expression profiles of the E14.5 control, β-cat^{Dermol}, and β-cat^{Dermol}ΔTF calvarial mesenchymes. The strategy was to reveal common differentially expressed genes (DEGs) affected by both β-cat^{Dermol} and β-cat^{Dermol}ΔTF mutations compared to the control. Next, unique DEGs specific to β-cat^{Dermol} was identified, thereby differentiating nonclassical β-catenin effects on cell fate switching from the classical β-catenin signaling linked to osteoblastogenesis (FIG. 5A). The downstream effectors of nonclassical signaling were uncovered by the subtraction of the β-cat^{Dermol}ΔTF from β-cat^{Dermol} DEGs to obtain a total of 431 DEGs potentially affected by the β-catenin-dependent but transcription-independent pathway (FIG. 5A).

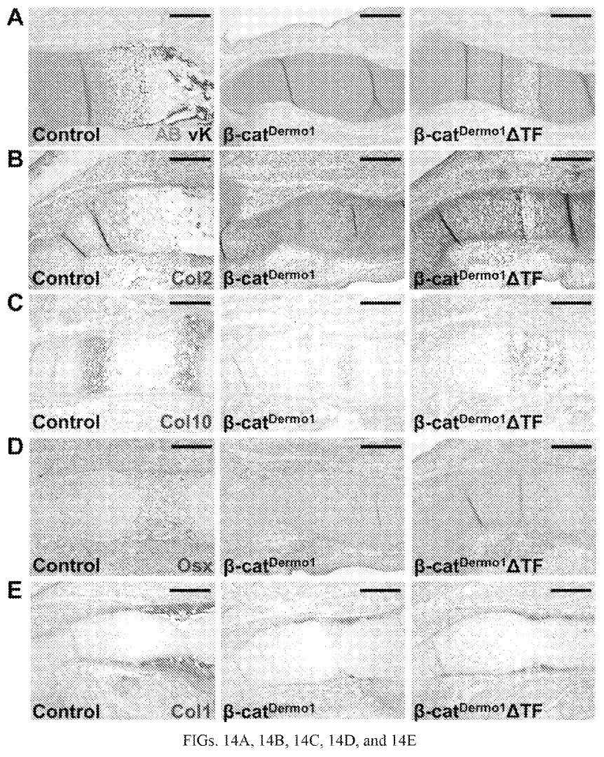

To gain mechanistic insight from the identified 1093 common DEGs. Gene Set Enrichment Analysis was performed using MetaCore (32). The pathway analysis mapped 7 out of the top 50 pathways related to Wnt signaling in tissue development and maintenance (FIG. 16A). As β-catenin functions as a cofactor for TCF transcription factors to mediate canonical Wnt signaling, next the effect of the mutations on the β-catenin/TCF-dependent downstream targets was examined (33). Comparative gene expression analysis revealed that 20 genes known to be directly regulated by β-catenin-LEF/TCF-dependent transcription were substantially decreased in β-cat^{Dermol} and β-cat^{Dermol}ΔTF mutants (FIG. 16B), including TCF7 and DKK1, confirmed by immunostaining analysis (FIG. 3E, F). The results showed a clear reduction of canonical Wnt signaling in the β-cat^{Dermol} and β-cat^{Dermol}ΔTF mesenchymes.

Next, the examination of osteoblast and chondrocyte markers showed that their expression coincides with skeletogenic abnormalities (FIG. 5B-C). The expression of osteoblast genes was dramatically downregulated in both β-cat^{Dermol} and β-cat^{Dermol}ΔTF mutants (FIG. 5B). The canonical Wnt/f-catenin signaling regulates osteoblast differentiation at very early stages as evidenced by the alteration of osteoprogenitor markers, Runx2 and Osx, and osteoblast markers. Col1a1, Ibsp, and OPN (FIG. 5B). Consistent with the alteration of skeletal lineage commitment, the chondrocyte genes were strongly elevated only in the calvaria of β-cat^{Dermol} (FIG. 5C). The disruption of the canonical Wnt/β-catenin pathway did not alter skeletal cell fate. These findings provide molecular profiling supports for the skeletal characterizations and suggest the suitable use of these sequencing datasets to further decipher the regulatory process associated with nonclassical β-catenin (transcription-independent) signaling effects on cell fate switching.

### Example 6 Identification of GATA3 Associated with Skeletal Cell Fate Switching

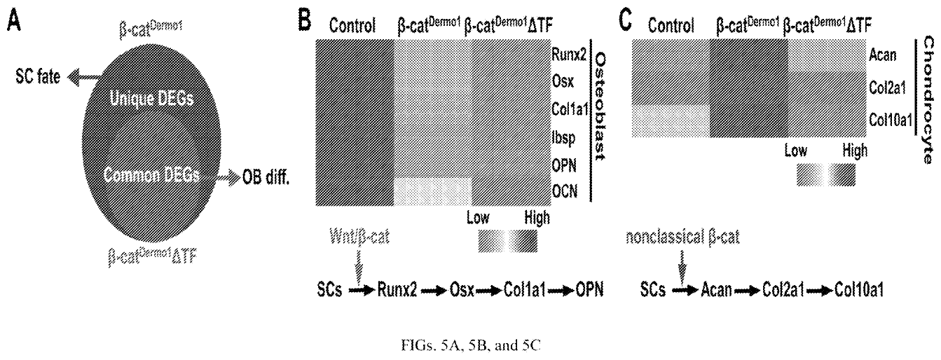

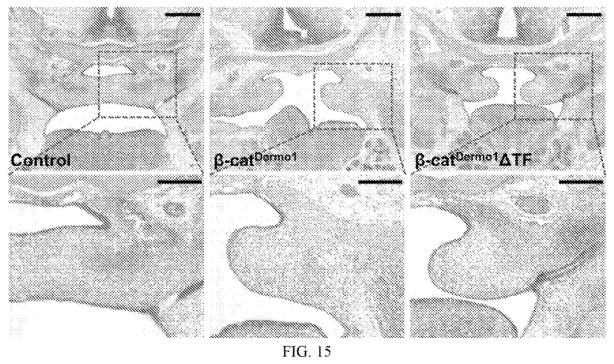

To elucidate the mechanism underlying nonclassical β-catenin signaling, inventors first revealed its primary effect by upstream regulator assay using MetaCore Interactome analysis (34). By analyzing the downstream “Effectors” (unique DEGs), the goal was to find upstream “Regulators” with dynamic effects on stem cell fate switching (FIG. 6A). Inventors were able to identify eleven key factors statistically over-connected within the downstream effectors (FIG. 17). GATA3 was identified as the top candidate linked to 24 network objects in unique DEGs. GATA3 elevation was predicted to promote the dynamic changes of the downstream effectors that lead to the alteration of skeletal lineage commitment (FIG. 6B). This prediction was further validated by an immunostaining study showing an increased expression of GATA3 in the β-cat^{Dermol} but not β-cat^{Dermol}ΔTF skeletogenic mesenchyme (FIG. 6C) as well as in the ectopic chondrocytes (FIG. 6D). The results suggest the link of GATA3 elevation to nonclassical β-catenin signaling in the cell fate alteration.

### Example 7 GATA3 is Sufficient to Alter Skeletal Lineage Commitment

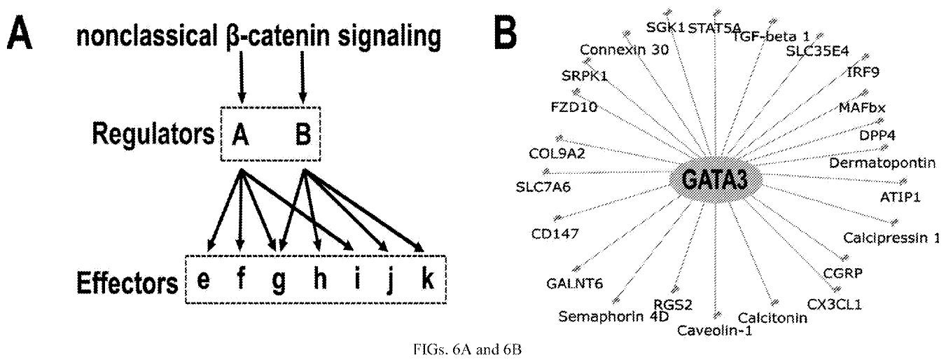

To determine the role of GATA3 in skeletogenic lineage specification, functional studies were performed in the cells and mice. First, C3H10T1/2 mesenchymal cells were infected with lentivirus expressing GFP (control) or GATA3 (Gata3^{OE}), followed by in vitro differentiation into chondrocytes. Alcian blue staining revealed that the average of positively stained areas in the Gata3^{OE} culture is two times more than the control (FIG. 7A).

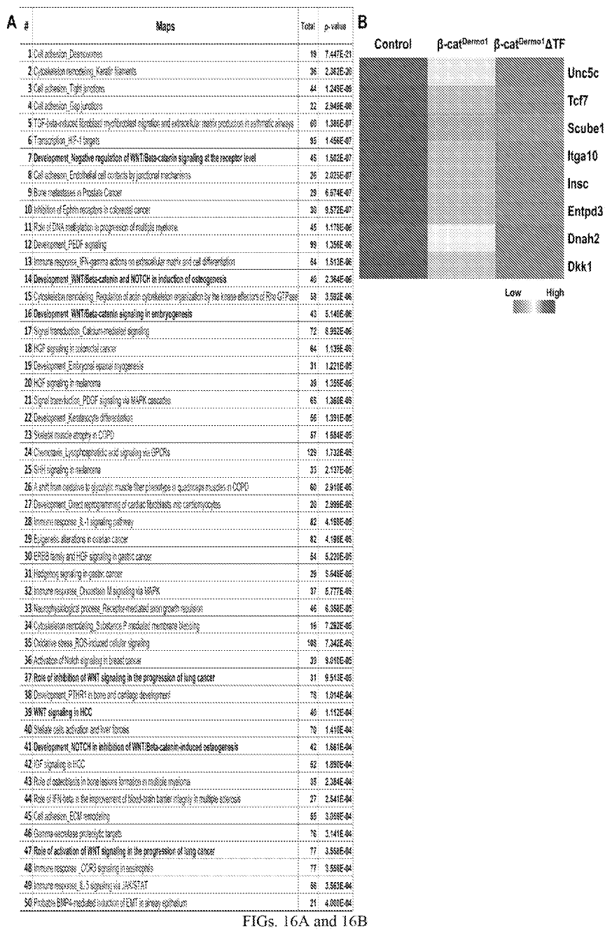

Next, the GATA3^{Dermol-OE} model was developed by crossing mice homozygous for the R26StopGATA3 allele with Dermol-Cre mice for mesenchymal expression of GATA3 (FIG. 18A). The transgenic expression of GATA3 was detected in craniofacial skeletogenic mesenchyme (FIG. 18B).

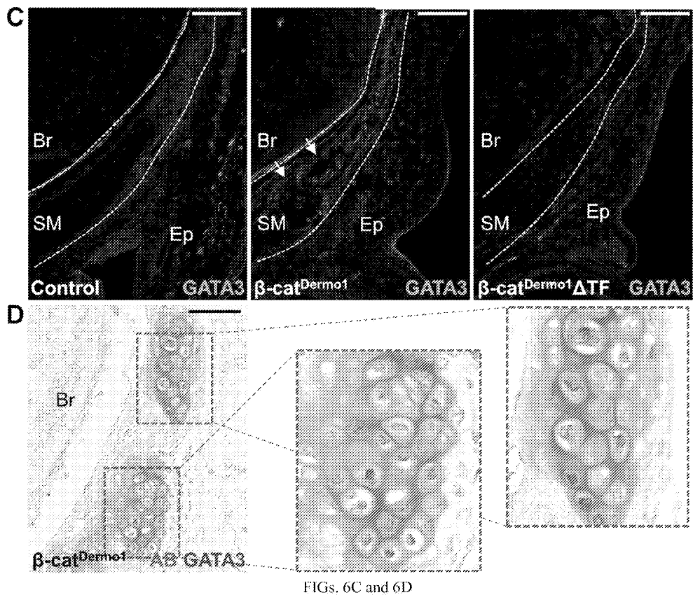

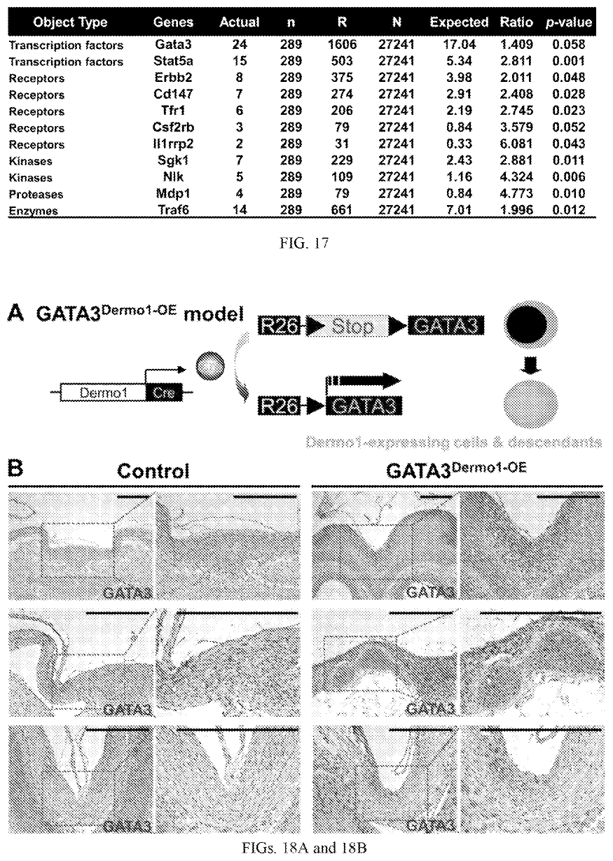

In GATA3^{Dermol-OE} mice, ectopic cartilages were detected in various regions of the skull (FIG. 7B, C, n≥3, 100% penetrance). In the calvaria suture mesenchyme, several areas displayed severe abnormalities in chondrogenesis (FIG. 8A-C, FIG. 19A-B; E15.5-18.5, n=24, 100% penetrance). Because calvarial bone formation was mediated by intramembranous ossification, only Osx+ cells were present in the control mesenchyme (FIG. 8A, B).

In the GATA3^{Dermol-OE} mice, cells expressing Aggrecan (Acan) were identified, indicating a switch of osteogenic to chondrogenic fate in the mesenchymal regions (FIG. 8A. B, and FIG. 19C-D). The anterior skull, containing nasal cartilage and bones, also exhibited a drastic expansion of the nasal cartilage in the Gata3^{Dermol-OE} mutant (FIG. 8C). These results provided definitive proof of GATA3 in skeletal cell fate switching.

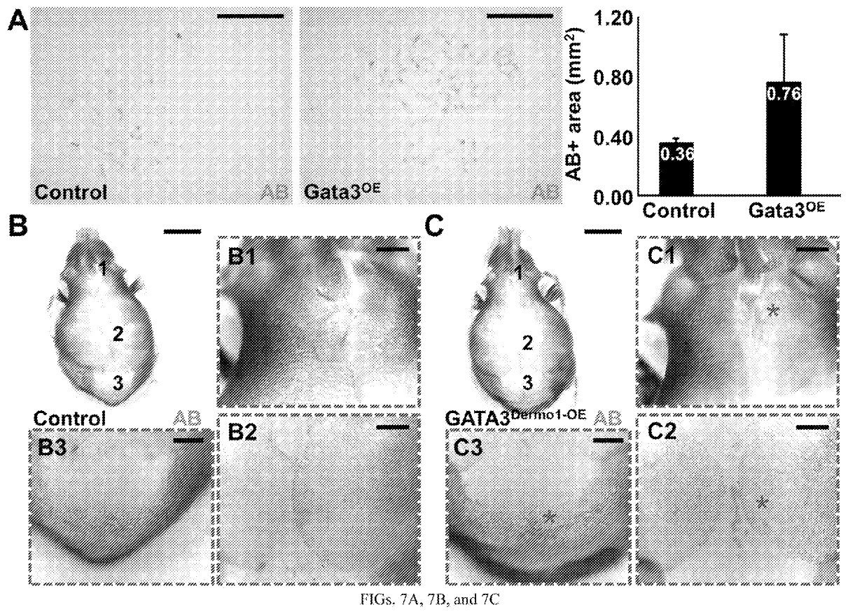

Next, a functional test was performed to determine the role of GATA3 in mediating nonclassical β-catenin signaling. The ex vivo culture of primary cells isolated from control and β-cat^{Dermol} showed the loss of β-catenin enhances chondrogenesis (FIG. 9A, B). However, the enhanced chondrogenesis of β-cat^{Dermol} was significantly alleviated by lentivirus-mediated knockdown of GATA3 (FIG. 9A, B, Mean±SD, n=3 animals, p-value<0.01, student t-test). The quantitative RT-PCR further revealed the elevated expression of chondrogenic markers significantly alleviated by the reduction of GATA3 (FIG. 9C, Mean±SD, n=3 animals, p-value<0.01, student t-test). The results thus suggested the dependence of GATA3 in the β-catenin-mediated commitment of skeletogenic lineage.

### Example 8 GATA3 Activators in Promoting Chondrogenesis

To further investigate the beneficial effects of GATA3 stimulation on cartilage regeneration, the inventors examined GATA3 activators in chondrogenesis. Using the ATDC5 cell line derived from mouse teratocarcinoma (undifferentiated embryonal carcinoma that can give rise to all three germ layers), the inventors first cultured the cells in micro-mass followed by differentiation into chondrocytes. Next, the inventors testes if the addition of GATA3 activators to the micro-mass culture could promote chondrogenesis. Staining was then performed to detect chondrogenic cells, which were positive for alcian blue. Imaging and quantitative analysis was then carried out to determine the percentage of the positively stained area using ImageJ. The inventors tested a few GATA3 activators including Docosahexaenoic acid (DHA), Pirinixic acid (aka WY-14643), Gemfibrozil (aka Lopid), SB 202190, SCH 58261, and U0126 that were known to enhance GATA3 expression (Attakpa, E., et al., Biochimie. 2009 91(11-12): p. 1359-65; Gocke, A. R., et al., J Immunol, 2009.PMC2959196 182(7): p. 4479-87, and Ahmad, S. F., et al., J Neuroimmunol, 2017 311: p. 59-67). DHA, an omega-3 fatty acid, has been shown to enhance GATA3 transcripts and proteins (Attakpa, E., et al., Biochimie, 2009 91(11-12); p. 1359-65).

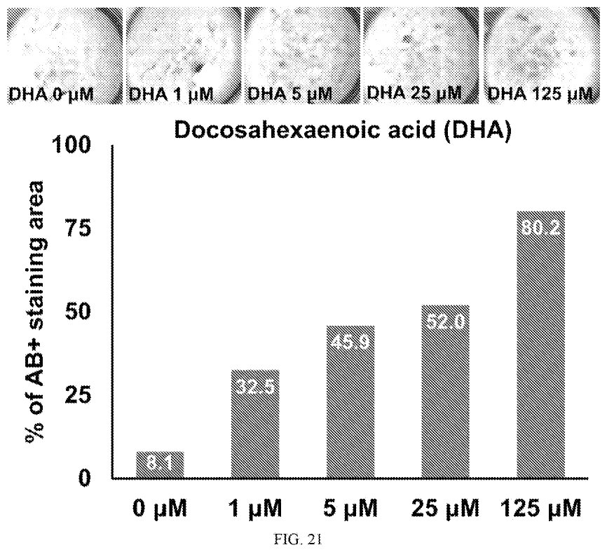

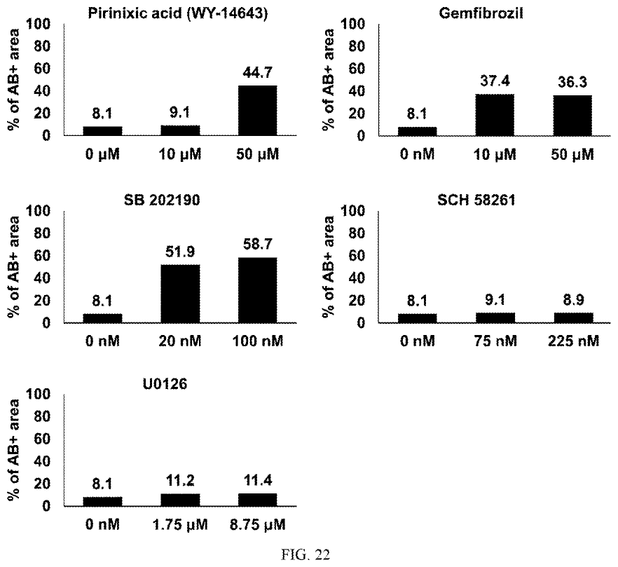

As shown in FIG. 21, the presence of DHA stimulated chondrocyte differentiation in a dosage-dependent manner. In addition, Pirinixic acid, Gemfibrozil, and SB202190 also exhibited substantial stimulated effects on chondrogenesis. See FIG. 22.

### Discussion

This study provides evidence that β-catenin signaling independent of its transcriptional function specifies skeletal cell fate. The loss of β-catenin-dependent transcription does not alter skeletal lineage commitment arguing against the previous knowledge where canonical Wnt signaling is required for skeletal fate determination. Based on genetic studies, it is proposed that nonclassical signaling mediated by β-catenin is essential for skeletal lineage commitment. Gene expression profiling and bioinformatics analyses further identify GATA3 to mediate the nonclassical signaling effect of β-catenin on skeletal cell fate determination. The importance of GATA3 in chondrogenic fate is further demonstrated by clear evidence from n vitro cell differentiation and in vivo transgenic animal studies. The programming of skeletal precursors is switched from an osteogenic to chondrogenic fate by the expression of GATA3 alone, suggesting it acts as a master regulator in skeletal lineage commitment.

The β-catenin-dependent nonclassical effects may include those of cell-cell interaction and LEF/TCF-independent transcription. The β-cat ΔTF mutant protein can associate with E-cadherin at the adherens junction and remains detectable in the nucleus upon Wnt stimulation, suggesting the mutation does not affect the subcellular distribution of β-catenin (35). The loss of Lrp5 and Lrp6 in mice develops extra cartilage elements (24) that seem to favor the involvement of β-catenin-mediated transcription independent of LEF/TCF over the cell adhesion function. Wnt signals mediated through β-catenin may generate transcriptional outputs distinct from the LEF/TCF of canonical signaling (33). The interaction of β-catenin with other transcription factors e.g., FOXO, HIF, and SOX17 via the armadillo repeats suggests such alternative downstream effects (36-38). β-catenin including β-cat ΔTF mutant may be a repressor directly or indirectly affecting GATA3. An interesting question is whether the β-catenin-GATA3 regulatory axis is modulated by Wnt. Mice with Wnt9a deficiency exhibit abnormal cartilage formation in the skull similar to those caused by overexpression of GATA3 (39). Wnt9a may exert nonclassical signaling effects through modulation of the β-catenin/GATA3 regulatory axis. However, it remains possible that the cell fate determination requires β-catenin-mediated cell adhesion in the craniofacial mesenchyme. Although the mechanism underlying nonclassical β-catenin signaling remains elusive, the role of GATA3 in skeletal cell fate determination is clear.

### REFERENCES

1. J. Rossant, J. C. Cross, Placental development: lessons from mouse mutants. Nat Rev Genet 2, 538-548 (2001).
2. K. L. Moore, T. V. N. Persaud. M. G. Torchia. C. Elsevier, The developing human: clinically oriented embryology (ClinicalKey, Elsevier, Edinburgh, ed. 11th edition, 2020).
3 Y. Takahashi. D. Sipp. H. Enomoto, Tissue interactions in neural crest cell development and disease. Science (New York, N.Y. 341, 860-863 (2013).
4. B. Plusa, A.-K. Hadjantonakis, Cell Fate in Mammalian Development (Current topics in developmental biology v. 128. Academic Press, an imprint of Elsevier, Cambridge, MA, ed. First edition, 2018).
5. B. R. Olsen, A. M. Reginato, W. Wang, Bone development. Annual review of cell and developmental biology 16, 191-220 (2000).
6. B. Sacchetti, A. Funari, S. Michienzi, S. Di Cesare, S. Piersanti, I. Saggio, E. Tagliafico, S. Ferrari, P. G. Robey, M. Riminucci, P. Bianco, Self-renewing osteoprogenitors in bone marrow sinusoids can organize a hematopoietic microenvironment. Cell 131, 324-336 (2007).
7. H. Zhao, J. Feng, T. V. Ho, W. Grimes, M. Urata, Y. Chai, The suture provides a niche for mesenchymal stem cells of craniofacial bones. Nature cell biology 17, 386-396 (2015).
8. C. K. Chan, E. Y. Seo, J. Y. Chen, D. Lo, A. McArdle, R. Sinha, R. Tevlin, J. Seita, J. Vincent-Tompkins. T. Wearda, W. J. Lu, K. Senarath-Yapa, M. T. Chung, O. Marecic, M. Tran, K. S. Yan, R. Upton, G. G. Walmsley, A. S. Lee, D. Sahoo, C. J. Kuo, I. L. Weissman, M. T. Longaker. Identification and specification of the mouse skeletal stem cell. Cell 160, 285-298 (2015).
9. D. L. Worthley, M. Churchill, J. T. Compton, Y. Tailor, M. Rao, Y. Si, D. Levin, M. G. Schwartz, A. Uygur, Y. Hayakawa, S. Gross, B. W. Renz, W. Setlik, A. N. Martinez, X. Chen. S. Nizami, H. G. Lee, H. P. Kang, J. M. Caldwell, S. Asfaha, C. B. Westphalen, T. Graham, G. Jin, K. Nagar, H. Wang, M. A. Kheirbek, A. Kolhe, J. Carpenter, M. Glaire, A. Nair, S. Renders, N. Manien, S. Muthupalani, J. G. Fox, M. Reichert, A. S. Giraud, R. F. Schwabe, J. P. Pradere, K. Walton, A. Prakash, D. Gumucio, A. K. Rustgi, T. S. Stappenbeck, R. A. Friedman, M. D. Gershon, P. Sims, T. Grikscheit, F. Y. Lee, G. Karsenty, S. Mukherjee, T. C. Wang, Gremlin 1 identifies a skeletal stem cell with bone, cartilage, and reticular stromal potential. Cell 160, 269-284 (2015).
10. S. Mender-Ferrer, T. V. Michurina, F. Ferraro, A. R. Mazloom, B. D. Macarthur. S. A. Lira, D. T. Scadden, A. Ma'ayan, G. N. Enikolopov, P. S. Frenette, Mesenchymal and haematopoietic stem cells form a unique bone marrow niche. Nature 466, 829-834(2010).
11. B. O. Zhou, R. Yue, M. M. Murphy, J. G. Peyer, S. J. Morrison, Leptin-receptor-expressing mesenchymal stromal cells represent the main source of bone formed by adult bone marrow. Cell Stem Cell 15, 154-168 (2014).
12. T. Maruyama, J. Jeong, T. J. Sheu, W. Hsu, Stem cells of the suture mesenchyme in craniofacial bone development, repair and regeneration. Nature communications 7, 10526 (2016).
13. P T. Newton, L. Li, B. Zhou, C. Schweingruber, M. Hovorakova, M. Xie, X. Sun, L. Sandhow, A. V. Artemov, E. Ivashkin, S. Suter, V. Dvachuk, M. El Shahawy, A. Gritli-Linde, T. Bouderlique, J. Petersen, A. Mollbrink, J. Lundeberg, G. Enikolopov, H. Qian, K. Fried, M. Kasper, E. Hedlund, I. Adameyko, L. Savendahl, A. S. Chagin, A radical switch in clonality reveals a stem cell niche in the epiphyseal growth plate. Nature 567, 234-238 (2019).
14. K. Mizuhashi, W. Ono, Y. Matsushita, N. Sakagami, A. Takahashi, T. L. Saunders, T. Nagasawa, H. M. Kronenberg, N. Ono. Resting zone of the growth plate houses a unique class of skeletal stem cells. Nature 563, 254-258 (2018).
15. T. Maruyama, R. Stevens, A. Boka, L. DiRienzo, C. Chang, H. I. Yu, K. Nishimori, C. Morrison. W. Hsu, BMPR1A maintains skeletal stem cell properties in craniofacial development and craniosynostosis. Sci Transl Med 13. (2021).
16. A. S. Brack, M. J. Conboy, S. Roy, M. Lee, C. J. Kuo, C. Keller, T. A. Rando, Increased Wnt signaling during aging alters muscle stem cell fate and increases fibrosis. Science (New York, N.Y 317, 807-810 (2(007).
17. S. Biressi, E. H. Miyabara, S. D. Gopinath, P. M. Carlig, T. A. Rando, A Wnt-TGFbeta2 axis induces a fibrogenic program in muscle stem cells from dystrophic mice. Sci Transl Med 6, 267ra176 (2014).
18. C. Meyers, J. Lisiecki, S. Miller, A. Levin, L. Fayad, C. Ding, T. Sono, E. McCarthy, B. Levi, A. W. James, Heterotopic Ossification: A Comprehensive Review. JBMR Plus 3, e10172 (2019).
19. E. M. Shore, F. S. Kaplan, Inherited human diseases of heterotopic bone formation. Nat Rev Rheumatol 6, 518-527 (2010).
20. E. M. Shore, M. Xu, G. J. Feldman, D. A. Fenstermacher, T. J. Cho, I. H. Choi, J. M. Connor, P. Delai, D. L. Glaser, M. LeMerrer, R. Morhart, J. G. Rogers, R. Smith, J. T. Triffitt, J. A. Urtizberea, M. Zasloff, M. A. Brown, F. S. Kaplan, A recurrent mutation in the BMP type I receptor ACVR1 causes inherited and sporadic fibrodysplasia ossificans progressiva. Nature genetics 38, 525-527 (2006).
21. T. Maruyama. A. J. Mirando, C. X. Deng, W. Hsu, The balance of WNT and FGF signaling influences mesenchymal stem cell fate during skeletal development. Sci Signal 3, ra40 (2010).
22. T. P. Hill. D. Spater, M. M. Taketo, W. Birchmeier, C. Hartmann, Canonical Wnt/beta-catenin signaling prevents osteoblasts from differentiating into chondrocytes. Developmental cell 8, 727-738 (2005).
23. T. F. Day, X. Guo, L. Garrett-Beal, Y. Yang, Wnt/beta-catenin signaling in mesenchymal progenitors controls osteoblast and chondrocyte differentiation during vertebrate skeletogenesis. Developmental cell 8, 739-750 (2005).
24. K. S. Joeng, C. A. Schumacher, C. R. Zylstra-Diegel, F. Long, B. O. Williams, Lrp5 and Lrp6 redundantly control skeletal development in the mouse embryo. Developmental biology 359, 222-229 (2011).
25. T. Maruyama, M. Jiang. W. Hsu. Gpr177, a novel locus for bone mineral density and osteoporosis, regulates osteogenesis and chondrogenesis in skeletal development. J Bone Miner Res 28, 1150-1159 (2013).
26. J. Fu, M. Jiang, A. J. Mirando, H. M. Yu, W. Hsu, Reciprocal regulation of Wnt and Gpr177/mouse Wntless is required for embryonic axis formation. Proceedings of the National Academy of Sciences of the United States of America 106, 18598-18603 (2009).
27. A. J. Mikels, R. Nusse. Purified Wnt5a protein activates or inhibits beta-catenin-TCF signaling depending on receptor context. PLoS biology 4, e115 (2006).
28. M. Bauer. K. Willert. Wnt signaling, the beta-cat(enin)'s meow. Genes & development 26, 105-109 (2012).
29. T. Valenta, M. Gay, S. Steiner, K. Draganova, M. Zemke, R. Hoffmans, P. Cinelli, M. Aguet, L. Sommer. K. Basler, Probing transcription-specific outputs of beta-catenin in vivo. Genes & development 25, 2631-2643 (2011).
   A. Ortiz, Y. C. Lee, G. Yu. H. C. Liu, S. C. Lin, M. A. Bilen, H. Cho, L. Y. Yu-Lee, S. H. Lin, Angiomotin is a novel component of cadherin-11/beta-catenin/p120 complex and is critical for cadherin-11-mediated cell migration. FASEB J 29, 1080-1091 (2015).
31. B. Liu, H. M. Yu. W. Hsu. Craniosynostosis caused by Axin2 deficiency is mediated through distinct functions of beta-catenin in proliferation and differentiation. Developmental biology 301, 298-308 (2007).
32. A. Subramanian, P. Tamayo, V. K. Mootha, S. Mukherjee, B. L. Ebert, M. A Gillette, A. Paulovich, S. L. Pomeroy, T. R. Golub, E. S. Lander, J. P. Mesirov, Gene set enrichment analysis: a knowledge-based approach for interpreting genome-wide expression profiles. Proceedings of the National Academy of Sciences of the United States of America 102, 15545-15550 (2005).
33. N. Doumpas, F. Lampart, M. D. Robinson, A. Lentini, C. E. Nestor, C. Cantu, K. Basler, TCF/LEF dependent and independent transcriptional regulation of Wnt/beta-catenin target genes. EMBO J 38, (2019).
34. S. Schuierer, L. C. Tranchevent, U. Dengler, Y. Moreau, Large-scale benchmark of Endeavour using MetaCore maps. Bioinformatics 26, 1922-1923 (2010).
35. D. Buechel, N. Sugiyama, N. Rubinstein, M. Saxena, R. K. R. Kalathur, F. Luond, V. Vafaizadeh, T. Valenta. G. Hausmann, C. Cantu. K. Basler, G. Christofori, Parsing beta-catenin's cell adhesion and Wnt signaling functions in malignant mammary tumor progression. Proceedings of the National Academy of Sciences of the United States of America 118, (2021).
36. M. A. Essers, L. M. de Vries-Smits, N. Barker, P. E. Polderman, B. M. Burgering, H. C. Korswagen, Functional interaction between beta-catenin and FOXO in oxidative stress signaling. Science (New York, N.Y 308, 1181-1184 (2005).
37. A. Kaidi, A. C. Williams, C. Paraskeva, Interaction between beta-catenin and HIF-1 promotes cellular adaptation to hypoxia. Nature cell biology 9, 210-217 (2007).
38. A. M. Zom, G. D. Barish, B. O. Williams, P. Lavender, M. W. Klymkowsky, H. E. Varmus, Regulation of Wnt signaling by Sox proteins: XSox17 alpha/beta and XSox3 physically interact with beta-catenin. Molecular cell 4, 487-498 (199)).
39. D. Spater, T. P. Hill, J. O'Sullivan R, M. Gruber, D. A. Conner, C. Hartmann, Wnt9a signaling is required for joint integrity and regulation of Ihh during chondrogenesis. Development (Cambridge, England) 133, 3039-3049 (2006).
40. M. Tremblay, O. Sanchez-Ferras, M. Bouchard, GATA transcription factors in development and disease. Development (Cambridge, England) 145. (2018).
41. P. P. Pandolfi, M. E. Roth, A. Karis, M. W. Leonard, E. Dzierzak, F. G. Grosveld, J. D. Engel, M. H. Lindenbaum, Targeted disruption of the GATA3 gene causes severe abnormalities in the nervous system and in fetal liver haematopoiesis. Nature genetics 11, 40-44 (1995)
42. K. C. Lim, G. Lakshmanan, S. E. Crawford, Y. Gu, F. Grosveld, J. D. Engel, Gata3 loss leads to embryonic lethality due to noradrenaline deficiency of the sympathetic nervous system. Nature genetics 25, 209-212 (2000).
43. H. Van Esch, P. Groenen, M. A. Nesbit, S. Schuffenhauer, P. Lichtner, G. Vanderlinden, B. Harding, R. Beetz, R. W. Bilous, I. Holdaway, N. J. Shaw, J. P. Fryns, W. Van de Ven, R. V. Thakker, K. Devriendt, GATA3 haplo-insufficiency causes human HDR syndrome. Nature 406, 419-422 (2000).
44. Y. B. Zhang, J. Hu, J. Zhang, X. Zhou, X. Li, C. Gu, T. Liu, Y. Xie, J. Liu, M. Gu, P. Wang, T. Wu, J. Qian, Y. Wang, X. Dong, J. Yu, Q. Zhang. Genome-wide association study identifies multiple susceptibility loci for craniofacial microsomia. Nature communications 7, 10605 (2016).
45. N. Zaidan, K. Ottersbach, The multi-faceted role of Gata3 in developmental haematopoiesis Open Biol 8, (2018).
46. K. S. Zaret, J. S. Carroll, Pioneer transcription factors: establishing competence for gene expression. Genes & development 25, 2227-2241 (2011).
47. D. Sosic, J. A Richardson, K. Yu, D. M. Omitz, E. N. Olson. Twist regulates cytokine gene expression through a negative feedback loop that represses NF-kappaB activity. Cell 112, 169-180 (2003).
48. H. R. Dassule, P. Lewis, M. Bei, R. Maas, A. P. McMahon, Sonic hedgehog regulates growth and morphogenesis of the tooth. Development (Cambridge, England) 127, 4775-4785 (2000).
49. J. Fu, H. M Ivy Yu, T. Maruyama, A. J. Mirando, W. Hsu, Gpr177/mouse Wntless is essential for Wnt-mediated craniofacial and brain development. Dev Dyn 240, 365-371 (2011).
50. J. Chen. X. Tu, E. Esen, K. S. Joeng, C. Lin, J. M. Arbeit, M. A. Ruegg, M. N. Hall, L. Ma, F. Long. WNT7B promotes bone formation in part through mTORC1. PLoS genetics 10, e1004145 (2014).
51. O. Nyabi, M. Naessens, K. Haigh, A. Gembarska, S. Goossens, M. Maetens, S. De Clercq, B. Drogat, L. Haenebalcke, S. Bartunkova, I. De Vos, B. De Craene, M. Karimi, G. Berx, A. Nagy, P. Hilson, J. C. Marine, J. J. Haigh, Efficient mouse transgenesis using Gateway-compatible ROSA26 locus targeting vectors and F1 hybrid ES cells. Nucleic acids research 37, e55 (2009).
52. V. Brault. R. Moore, S. Kutsch, M. Ishibashi, D. H. Rowitch, A. P. McMahon, L. Sommer, O. Boussadia, R. Kemler, Inactivation of the beta-catenin gene by Wnt1-Cre-mediated deletion results in dramatic brain malformation and failure of craniofacial development. Development (Cambridge, England) 128, 1253-1264. (2001).
53. M. Bosma, W. Schuler, G. Bosma, The scid mouse mutant. Curr Top Microbiol Immunol 137, 197-202 (1988).
54. J. Fu, W. Hsu, Epidermal Wnt controls hair follicle induction by orchestrating dynamic signaling crosstalk between the epidermis and dermis. J Invest Dermatol 133, 890-898 (2013).
55. T. Maruyama, H. I. Yu. W. Hsu, Skeletal Stem Cell Isolation from Cranial Suture Mesenchyme and Maintenance of Stemness in Culture. Bio Protoc 12, e4339 (2022).
56. H. M. Yu, B. Jerchow, T. J. Sheu, B. Liu, F. Costantini, J. E. Puzas, W. Birchmeier, W. Hsu, The role of Axin2 in calvarial morphogenesis and craniosynostosis. Development (Cambridge, England) 132, 1995-2005 (2005).
57. T. Maruyama, M. Jiang, A. Abbott, H. I. Yu, Q. Huang, M. Chrzanowska-Wodnicka, E. I. Chen, W. Hsu, Rap1b is an effector of Axin2 regulating crosstalk of signaling pathways during skeletal development. J Bone Miner Res, (2017).
58. H. M. Yu, B. Liu, S. Y. Chiu, F. Costantini, W. Hsu, Development of a unique system for spatiotemporal and lineage-specific gene expression in mice. Proceedings of the National Academy of Sciences of the United States of America 102, 8615-8620 (2005).
59. H. I. Yu, T. Hsu, E. O. Maruvama, W. Paschen, W. Yang, W. Hsu. The requirement of SUMO2/3 for SENP2 mediated extraembryonic and embryonic development. Dev Dyn, (2010).
60. S. Y. Chiu, N. Asai, F. Costantini, W. Hsu. SUMO-Specific Protease 2 Is Essential for Modulating p53-Mdm2 in Development of Trophoblast Stem Cell Niches and Lineages. PLoS biology 6, e310 (2008).

The foregoing examples and description of the preferred embodiments should be taken as illustrating, rather than as limiting the present invention as defined by the claims. As w % ill be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method of generating a chondrocyte cell, comprising increasing a level of GATA3 in a non-chondrocyte cell.

2. The method of claim 1, wherein said increasing comprises:
(i) contacting the non-chondrocyte cell with a GATA3 activator, or
(ii) introducing to the non-chondrocyte cell a genetic construct comprising a nucleic acid sequence encoding GATA3, or
(iii) introducing to the non-chondrocyte cell a GATA3 polypeptide.

3. The method of claim 1, wherein the non-chondrocyte cell is a mesenchymal stem cell (MSC), a suture stem cell (SuSC), a skeletal stem cell (SSC), or a bone cartilage and stomal progenitor (BCSP).

4. The method of claim 1, wherein the non-chondrocyte cell is a somatic cell, a fibroblast, a skin cell, a muscle cell, an epithelium cell, a blood cell, a neuron, an embryonic cell, or a pluripotent stem cell (iPSC).

5. The method of claim 1, further comprising culturing the non-chondrocyte cell or a progeny thereof under conditions permitting differentiation of chondrocyte to obtain one or more progeny cells thereof, thereby generating chondrocyte cells.

6. A cell obtained according to the method of claim 1, or a progeny cell thereof.

7. A cultured recombinant cell comprising a heterologous GATA3 polypeptide or a heterologous nucleic acid encoding a GATA3 polypeptide, or a progeny cell thereof.

8. A composition comprising (i) the cell of claim 6, or a cell derived therefrom, and (ii) a carrier.

9. The composition of claim 8, wherein the composition is a pharmaceutical composition and the carrier is a pharmaceutically acceptable carrier.

10. A cartilage regeneration product or an artificial cartilage comprising (i) the cell of claim 6 or progeny thereof, and (ii) a scaffold.

11. A method of generating or regenerating a cartilaginous tissue at a site in a subject in need thereof, comprising:
(i) increasing a level of GATA3 in one or more cells at or around the site; or
(ii) administering to the site the cell of claim 7 or progeny thereof, or a composition comprising the cell or progeny thereof, or a cartilage regeneration product or an artificial cartilage comprising the cell or progeny thereof and a scaffold.

12. A method of treating a cartilage damage or of regenerating cartilage at a site in a subject in need thereof, comprising
(i) increasing a level of GATA3 in one or more cells at or around the site; or
(ii) administering to the site the cell of claim 7 or progeny thereof, or a composition comprising the cell or progeny thereof, or a cartilage regeneration product or an artificial cartilage comprising the cell or progeny thereof and a scaffold.

13. The method of claim 11, wherein the subject has arthritis or an injury at the site.

14. (canceled)

15. The method of claim 12, wherein the method enhances healing of the cartilage damage or prohibits development of fibrous connective tissue or fibrosis.

16. The method of claim 11, wherein said increasing comprises administering to the site a GATA3 activator, or a genetic construct comprising a nucleic acid sequence encoding GATA3, or a GATA3 polypeptide.

17. The method of claim 2, wherein the GATA3 activator is selected from the group consisting of Gemfibrozil, rosiglitazone, WY-14643, SB202190, docosahexaenoic acid (DHA), and Jagged1.

18. The method of claim 2, wherein the GATA3 polypeptide is linked to a cell-penetrating moiety.

19. The method of claim 11, further comprises administering to the subject a chondrogenic factor.

20. (canceled)

21. The method of claim 11, wherein the subject is a human.

22. The method of claim 11, wherein the cell is heterologous, xenogenic, allogeneic, isogenic, or autologous to the subject.

23. A kit for generating/regenerating a cartilaginous tissue, or for treating a cartilage damage, comprising one, two, or more selected from group consisting of:
a GATA3 activator,
a genetic construct comprising a nucleic acid sequence encoding GATA3,
a GATA3 polypeptide,
the cell of claim 7 or progeny thereof,
a composition comprising the cell or progeny thereof, and
a cartilage regeneration product or an artificial cartilage comprising the cell or progeny thereof and a scaffold.

24. (canceled)


SEQUENCE LISTING (15 sequences)

SEQ ID NO: 1 - protein, 318 aa, Homo sapiens
GLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKL

SEQ ID NO: 2 - DNA, 2306 bp, Homo sapiens
  source 1..2306: mol_type = genomic DNA
gcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccatacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaaaaaaaaaaaaaaaaaa

SEQ ID NO: 3 - protein, 443 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 4 - DNA, 3083 bp, Homo sapiens
  source 1..3083: mol_type = genomic DNA
gaacactgagctgcctggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaacta

SEQ ID NO: 5 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 6 - DNA, 2650 bp, Homo sapiens
  source 1..2650: mol_type = genomic DNA
gcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggc

SEQ ID NO: 7 - protein, 443 aa, Homo sapiens; identical to SEQ ID NO: 3

SEQ ID NO: 8 - DNA, 975 bp, Homo sapiens
  source 1..975: mol_type = genomic DNA
ggggcgcctcggagccgcgtgccctccgccccggggtgcccattgcgcagagcgtggcctggagacccgcgagccggggaaggtcgccgtggagtcccgaccagaggccggggttggggtcggtgcagaccgagggctggtttccttgactgtgggagaaacgccgggagccggagtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcgggggg

SEQ ID NO: 9 - protein, 143 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSG

SEQ ID NO: 10 - DNA, 748 bp, Homo sapiens
  source 1..748: mol_type = genomic DNA
gcggtgagcagttccagcggccaggacggcagcggcggccacagtggcgaactctgcctgtcatttctgccttcagatctccgggcgagtcaggaaaaaaaataaaaaacagctggccctcgggagcgagctgcccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacg

SEQ ID NO: 11 - protein, 80 aa, Homo sapiens
MEVTADQPRWVSHHHPAVLNGQHPDTHHPGLSHSYMDAAQYPLPEEVDVLFNIDGQGNHVPPYYGNSVRATVQRYPPTHH

SEQ ID NO: 12 - DNA, 3215 bp, Mus musculus
  source 1..3215: mol_type = genomic DNA
agctgtctgcgaacactgagctgcctggcgccgtcttgatagtttcagaaagaatgcattccctgtaaaaaaaaaatactgagagagggagaggagaaagagagagagactgagagagcgagacatagagagctacgcaatctgaccgggcaggtcacacgcctcctcctcctcctctacgctccttgctactcaggtgatcggaagagcaaccgtctctgagcgccaaggaatcagtgtgcagtgtggtcacactcggattcctctctccctcctttttttttttttttttttgacccctttattcctccgtgtctgctttttttttggggggggggatcgccctcattcttttctttttcttctttcccttcctttcttttgctaaactatcccgcaaagatttttctttcctccctaaaccctcctttttgctctccttttctatacccttaactgcaaacaaaccattaaacgacccctctcctgggcctccgacggcaggagtccgcggacctcccaggccgacagccctccctctacccgcgagggttccgggccgggcgagagggcgcgagcacagccgaggacatggaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggccggccactccagtcctcatctcttcaccttcccgcccaccccgccgaaagacgtctccccagacccgtcgctgtccaccccgggatccgccgggtcggccaggcaagatgagaaagagtgcctcaagtatcaggtgcagctgccagatagcatgaagctggagacgtctcactctcgaggcagcatgaccaccctgggtggggcctcatcctcagcccaccaccccattaccacctatccgccctatgtgcccgagtacagctctggactcttcccacccagcagcctgctgggaggatcccctaccgggttcggatgtaagtcgaggcccaaggcacgatccagcacagaaggcagggagtgtgtgaactgcggggcaacctctaccccactgtggcggcgagatggtaccgggcactacctttgcaatgcctgcggactctaccataaaatgaatgggcagaaccggccccttatcaagcccaagcgaaggctgtcggcagcaaggagagcagggacatcctgcgcgaactgtcagaccaccaccaccaccctctggaggaggaacgctaatggggacccggtctgcaatgcctgtgggctgtactacaagcttcataatattaacagacccctgactatgaagaaagaaggcatccagacccgaaaccggaagatgtctagcaaatcgaaaaagtgcaaaaaggtgcatgacgcgctggaggacttccccaagagcagctccttcaacccggccgctctctccagacacatgtcatccctgagccacatctctcccttcagccactccagccacatgctgaccacaccgacgcccatgcatccgccctccggcctctccttcggacctcaccacccttccagcatggtcaccgccatgggttagagaggcagagccctgctccacatgcgtgaggagtctccaagtgtgcgaagagttcctccgaccccttctacttgcgtttttcgcaggagcagtatcatgaagcccgaaagcgacagatctgtgtttttgaaggcagaaagcaaaatgtttgcttcttttttcaaaggagctcgaggtggtgtctgcattccaaccactgaatccggatcccatttgtgaataagccattcagactcatattccctatttaacagggtctctagtgctgtgaaaaaaatattgctgaacattgcatataacttatattgtaagaaatactgtacatttgaggaagactttattgtacctggatagctgtaagaaaggcatgaaggacgccaagagttttaaggaatatagggggattaaagtatggagatacagaagaaaccactaagtctgatgtccaaatgggcacactgtcagttttgtttcccttcagttgtttgatgcatttaaaaaaaaaaaaaagaaagaaaaagaaaaaaaggggggggggggagaaaaaaataaattaaaaaaaaaaaaaaaaaaagaaaagaaagaaaaatctaagaaaaaaaaaaaaaaggttgtaggcaaatcatttgttccaggctgtgagcctgtgcaaaagagatttcagatctgggcaatgggtgtgtgatctcacccactgaagatctgagaatgtcatggctaggcctacatgctctgtgaatcagtccctgtaattgttgtttgtatgtataattcagaagcaccaaaataagaaaagatgtagatttatttcatcatattatacagactgaattgttgtataaatttatttactgctagtgttaggaactgcttttttttttttttttggttttaatgttttttttttttttgttttttgtttttttttttctttctctctggatttttggttgaataaactagattgctttcagttgacttaaggtggatgtactctggagggtttatttttccttttattattatttttgatggtatttattaaatagcttctatgggcccggcggtacctgtctttttcgtcacttttcttgcagcctaaactatgaaggtagcagcgtaccagctaccaacatgcatgtcagagacccggccactcacaggcctggtcctgagagccacctggctgactgttagcccctgtgtgttctgtattagtgatcactgcctttaaacagtctgttggaataatactataaaaataataataaagttaaaatattttaaaacaa

SEQ ID NO: 13 - protein, 443 aa, Mus musculus
MEVTADQPRWVSHHHPAVLNGQHPDTHHPGLGHSYMEAQYPLTEEVDVLFNIDGQGNHVPSYYGNSVRATVQRYPPTHHGSQVCRPPLLHGSLPWLDGGKALSSHHTASPWNLSPFSKTSIHHGSPGPLSVYPPASSSSLAAGHSSPHLFTFPPTPPKDVSPDPSLSTPGSAGSARQDEKECLKYQVQLPDSMKLETSHSRGSMTTLGGASSSAHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDALEDFPKSSSFNPAALSRHMSSLSHISPFSHSSHMLTTPTPMHPPSGLSFGPHHPSSMVTAMG

SEQ ID NO: 14 - DNA, 674 bp, Mus musculus
  source 1..674: mol_type = genomic DNA
agtgttcaatccggagccggtgtcggcagaagggaaggagcgcggcgaggccggcagagcagacccgaggagacctgggggacagaaggtgctcgcgagggagctttagtaagaggagtgggagagggaagaaagtctgctttggttcggaagtgcccctataggacgtacattctaaggaaaaagaggctttcaaattggaggcgaacttgttgaaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggccggccactccagtcctcatctcttcaccttcccgccc

SEQ ID NO: 15 - protein, 119 aa, Mus musculus
MEAQYPLTEEVDVLFNIDGQGNHVPSYYGNSVRATVQRYPPTHHGSQVCRPPLLHGSLPWLDGGKALSSHHTASPWNLSPFSKTSIHHGSPGPLSVYPPASSSSLAAGHSSPHLFTFPP